(19) United States
(12) Patent Application Publication
(10) Pub. No.: US 2011/0112086 A1
(43) Pub. Date: May 12, 2011

(54) PYRIDINONE ANALOGS

(75) Inventors: Fabrice Pierre, La Jolla, CA (US); Michael Schwaebe, San Diego, CA (US)

(73) Assignee: CYLENE PHARMACEUTICALS, INC., San Diego, CA (US)

(21) Appl. No.: 12/303,952

(22) PCT Filed: Jun. 8, 2007
(86) PCT No.: PCT/US07/70774

§ 371 Date: Aug. 31, 2010

Related U.S. Application Data
(60) Provisional application No. 60/811,990, filed on Jun. 8, 2006.

(51) Int. Cl. A61K 31/437; C07D 495/14; A61K 31/5377; A61P 35/00

(52) U.S. Cl. 514/232.8; 546/65; 544/125; 514/287

(57) ABSTRACT

The present invention provides pyridinone analogs which may inhibit cell proliferation and/or induce cell apoptosis. The present invention also provides methods of preparing pyridinone analogs, and methods of using the same.

### FIELD OF THE INVENTION

The invention relates to pyridinone analogs and uses thereof. The invention also relates to methods of preparing pyridinone analogs.

### BACKGROUND

Evidence suggests quadruplex structures can exist in vivo in specific regions of the genome, including the telomeric ends of chromosomes and oncogene regulatory regions (Han, et al., Trends Pharm. Sci. (2000) 21:136-142). Quadruplex structures can form in purine-rich strands of nucleic acids. In duplex nucleic acids, certain purine rich strands are capable of engaging in a slow equilibrium between a typical duplex helix structure and in unwound and non-B-form regions. These unwound and non-B forms can be referred to as “paranemic structures.” Some forms are associated with sensitivity to 51 nuclease digestion, which can be referred to as “nuclease hypersensitivity elements” or “NHEs.” A quadruplex one type of paranemic structure and certain NHEs can adopt a quadruplex structure.

### SUMMARY OF THE INVENTION

The present invention provides pyridinone analogs which inhibit cell proliferation and/or induce cell apoptosis, and are believed to act by inhibition of ribosomal RNA biogenesis. The present invention also provides methods of preparing pyridinone analogs, and methods of using the same.

In one aspect, the present invention provides compounds having the general formula:

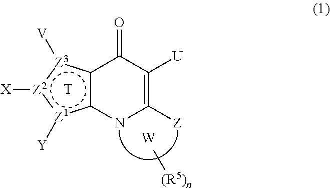

and pharmaceutically acceptable salts, esters and prodrugs thereof;

wherein V, X, and Y are absent if attached to a heteroatom other than Nitrogen, and independently H, halo, azido, R², CH₂R², SR², OR² or NR¹R² when attached to C or N; or

wherein V and X, or X and Y may form a carbocyclic ring, heterocyclic ring, aryl or heteroaryl, each of which may be optionally substituted and/or fused with a cyclic ring;

Z¹, Z² and Z³ are C, N, O or S, wherein among Z¹, Z² and Z³ there is at most one O atom and at most one S atom, and at most two carbon atoms;

Z is O, S, NR², CH₂ or C═O;

W together with N and Z forms an optionally substituted 5- or 6-membered ring that is fused to an optionally substituted aryl or heteroaryl, wherein said aryl or heteroaryl may be monocyclic or fused with a single or multiple ring, and wherein said ring optionally contains a heteroatom;

U is C(O)R², C(O)OR², C(O)NR¹R², C(O)NR¹—(CR¹₂)ₙ—NR³R⁴, SO₃R², SO₂NR¹R² or SO₂NR¹—(CR¹₂)ₙ—NR³R⁴;

wherein in each NR¹R², R¹ and R² together with N may form an optionally substituted ring;

in NR³R⁴, R³ and R⁴ together with N may form an optionally substituted ring;

R¹ and R³ are independently H or C₁₋₆ alkyl;

each R² is H, or a C₁₋₁₀ alkyl or C₂₋₁₀ alkenyl each optionally substituted with a halogen, one or more non-adjacent heteroatoms selected from N, O and S, a carbocyclic ring, a heterocyclic ring, an aryl or heteroaryl, wherein each ring is optionally substituted; or R² is an optionally substituted carbocyclic ring, heterocyclic ring, aryl or heteroaryl; or R² is COR¹ or S(O)ₓR¹ wherein x is 1-2;

R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;

each R⁵ is a substituent at any position on W; and is H, OR², amino, alkoxy, amido, halogen, cyano or an inorganic substituent; or R⁵ is C₁₋₆ alkyl, C₂₋₆ alkenyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring, optionally fused to an additional optionally substituted carbocyclic or heterocyclic ring; and

n is 1-6.

In the above formula (1), the ring labeled “T” is a five membered ring that can contain up to three heteroatoms selected from N, O, and S. Substituents V, X, and Y are as defined above, and each of them may be absent when the ring atom to which it is connected has no available open valence for substitution. The dashed circle indicates that each ring atom of ring T has a pi bond, which may be provided by either a heteroatom or an sp² hybridized carbon. In many embodiments, T is an aromatic ring, and in certain embodiments, T can be a non-aromatic ring. Ring “T” may, in some embodiments, form an optionally substituted 5-membered ring selected from the group consisting of:

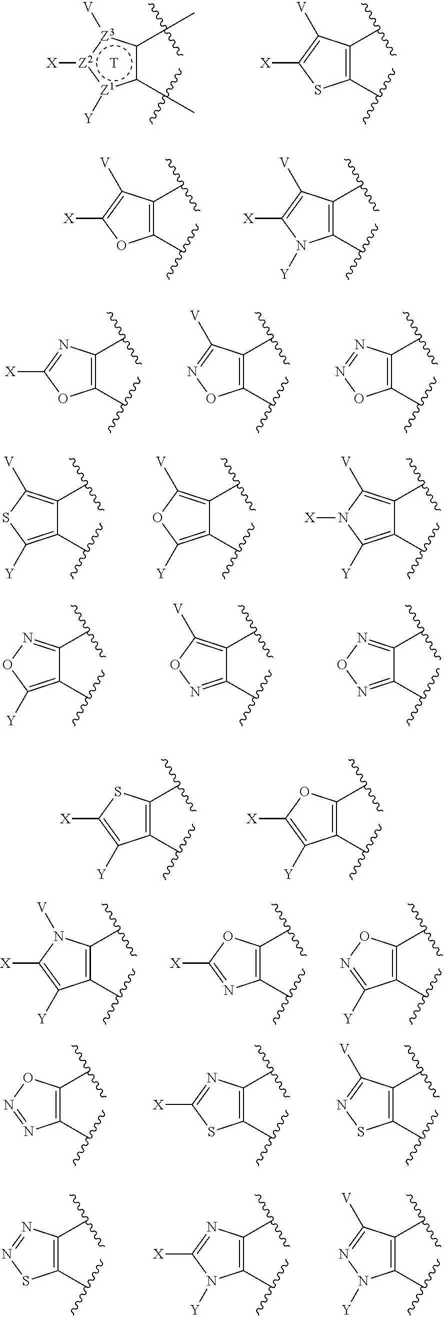

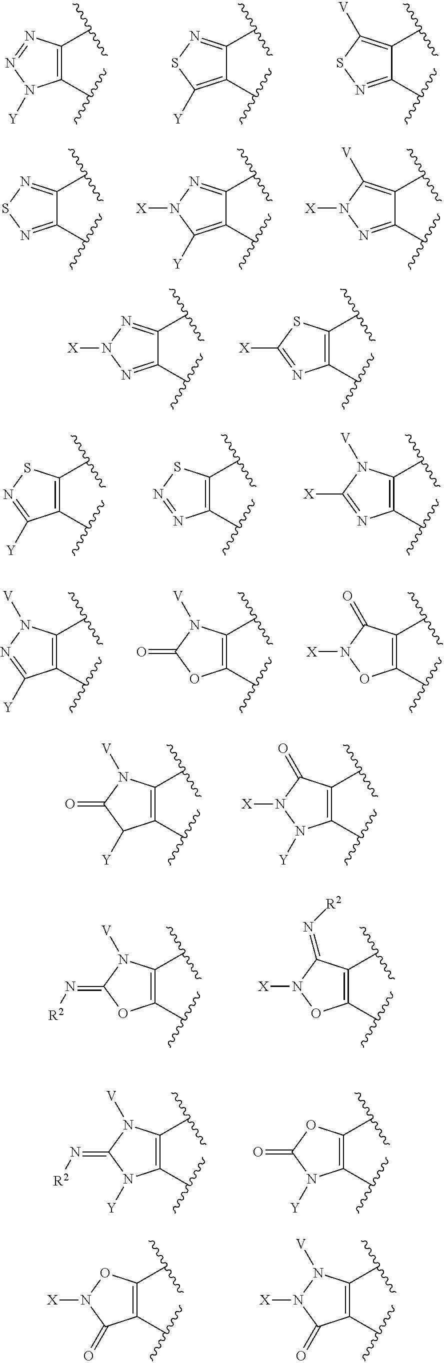

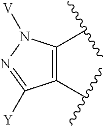

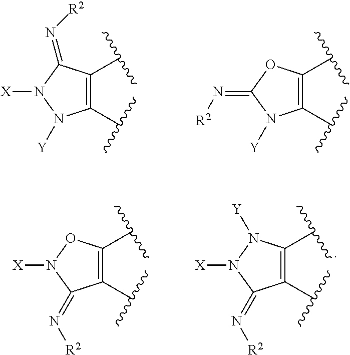

In the above formula (1), W together with N and Z may form an optionally substituted 5- or 6-membered aryl or heteroaryl ring that is fused to an optionally substituted aryl or heteroaryl selected from the group consisting of:

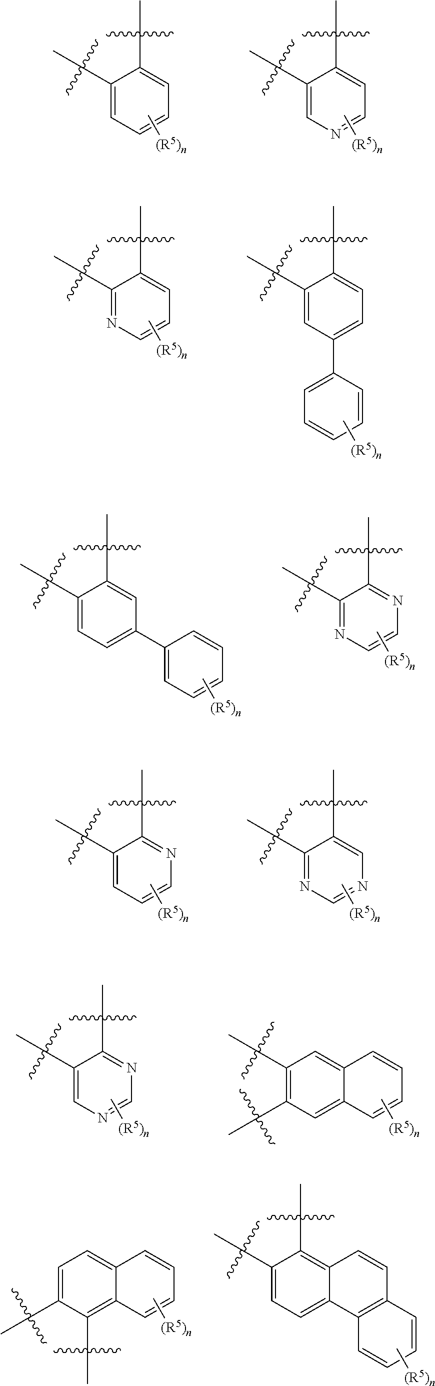

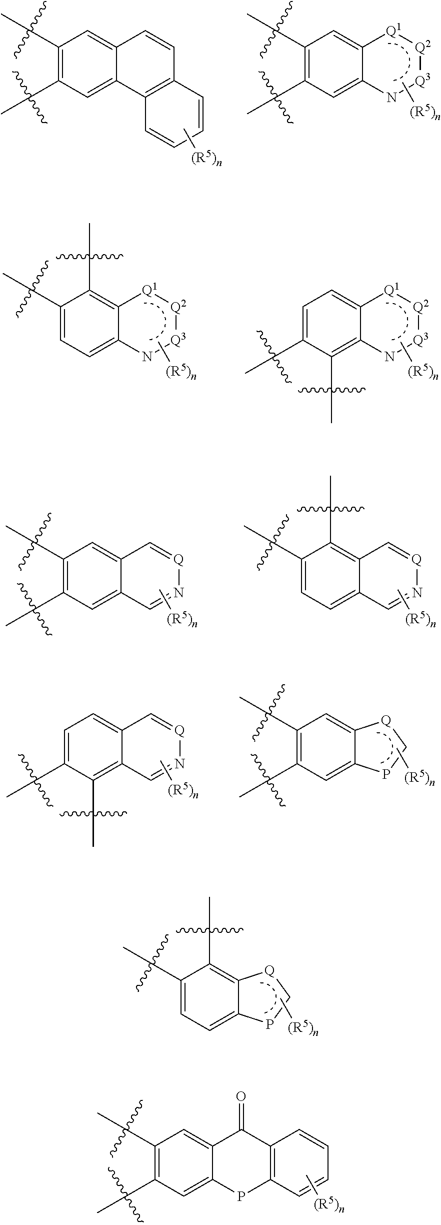

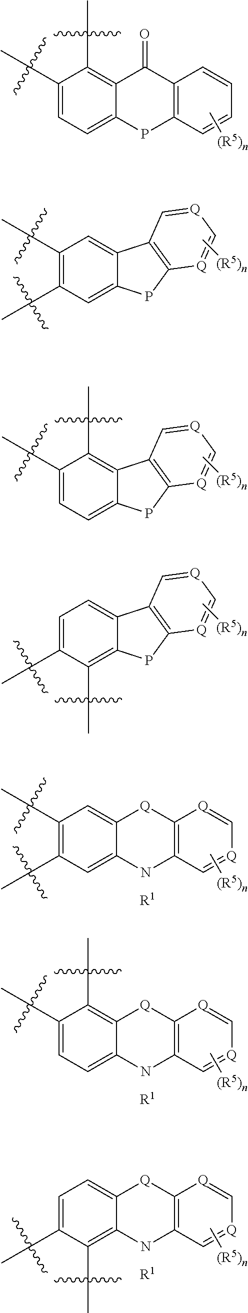

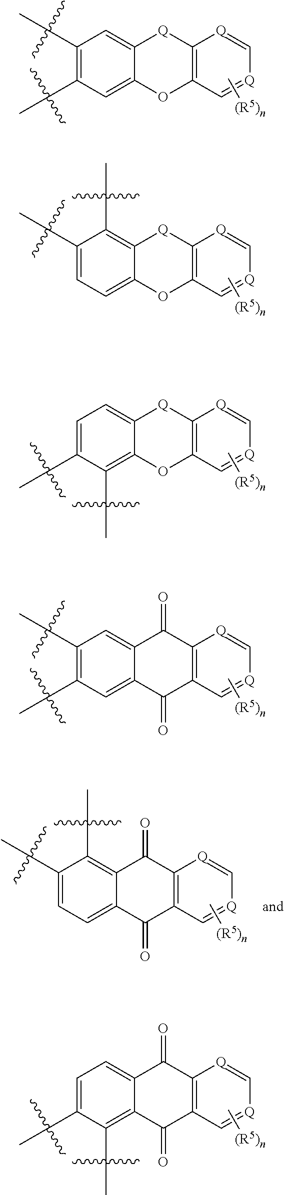

wherein each Q, Q¹, Q², and Q³ is independently CH or N;

P is independently O, CH, C═O or NR¹;

n and R⁵ is as defined above.

In other embodiments, W together with N and Z may form a group having the formula selected from the group consisting of

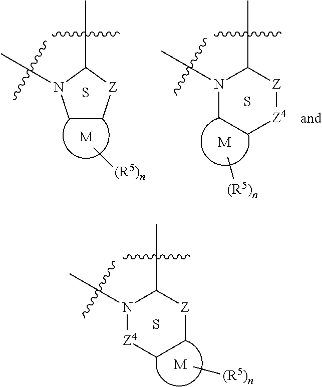

wherein Z is O, S, NR², CH₂ or C═O;

each Z⁴ is CR⁶, NR², or C═O;

R⁶ is H, or a substituent known in the art, including but not limited to hydroxyl, alkyl, alkoxy, halo, amino, or amido; and

Ring S and M may be saturated or unsaturated.

In some embodiments, W together with N and Z may form a 5- or 6-membered ring that is fused to a phenyl.

In yet another embodiment, the compounds of the present invention have the general formula (2A) or (2B):

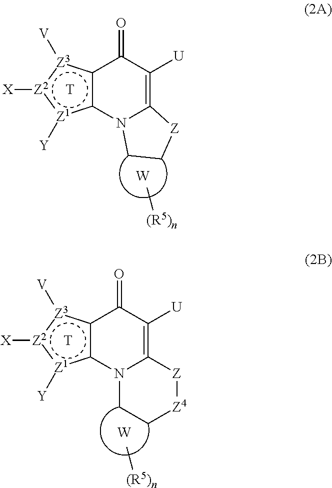

wherein U, V, W, X, Y, Z, Z¹, Z², Z³, R⁵ and n are as described above;

Z⁴ is CR⁶, NR², or C═O; and

Z and Z⁴ may optionally form a double bond.

In the above formula (1), (2A) and (2B), U may be C(O)NR¹R² or SO₂NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S, where the C₃₋₆ cycloalkyl, aryl or a 5-14 membered heterocyclic ring is optionally substituted. For example, R² may be a C₁₋₁₀ alkyl substituted with an optionally substituted morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodithiazole.

In other embodiments, U is SO₂NR¹—(CR¹₂)ₙ—NR³R⁴; n is 1-4; each R¹ is H or alkyl; and R³ and R⁴ in NR³R⁴ together form an optionally substituted piperidine, pyrrolidine, piperazine, morpholine, thiomorpholine, imidazole, or aminodithiazole. In some examples, U is SO₂NH—(CH₂)ₙ—NR³R⁴ wherein R³ and R⁴ together with N form an optionally substituted pyrrolidine, which may be linked to (CH₂)ₙ at any position in the pyrrolidine ring. In one embodiment, R³ and R⁴ together with N form an N-methyl substituted pyrrolidine.

In one embodiment, the present invention provides compounds having formula (1), (2A) or (2B), wherein:

each of V and Y if present is independently H or halogen (e.g., chloro or fluoro);

X is —(R⁵)R¹R², wherein R⁵ is C or N and wherein in each —(R⁵)R¹R², R¹ and R² together may form an optionally substituted aryl or heteroaryl ring;

Z is NH or N-alkyl (e.g., N—CH₃);

W together with N and Z forms an optionally substituted 5- or 6-membered ring that is fused with an optionally substituted aryl or heteroaryl ring; and

U is —SO₂R⁵R⁶—(CH₂)ₙ—CHR²—NR³R⁴, wherein R⁵ is CR¹ or N; R¹ is H or alkyl; R⁶ is H or C₁₋₁₀ alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In another embodiment, the present invention provides compounds having formula (1), (2A) or (2B), wherein:

V and Y if present is H or halogen (e.g., chloro or fluoro);

X if present is —(CR¹)R¹R², or NR¹R² and wherein R¹ and R² together may form an optionally substituted aryl or heteroaryl ring;

Z is NH or N-alkyl (e.g., N—CH₃);

W together with N and Z forms an optionally substituted 5- or 6-membered ring that is fused with an optionally substituted aryl or heteroaryl ring; and

U is —SO₂CR¹R⁶—(CH₂)ₙ—CHR²—NR³R⁴, or U is SO₂NR⁶—(CH₂)ₙ—CHR²—NR³R⁴

R⁶ is H or alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In yet another embodiment, the compounds of the present invention have the general formula (3):

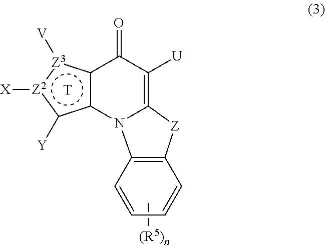

wherein U, V, X, Y, Z, Z¹, Z², Z³, R⁵ and n are as described above.

In yet another embodiment, the compounds of the present invention have the general formula (4A) or (4B):

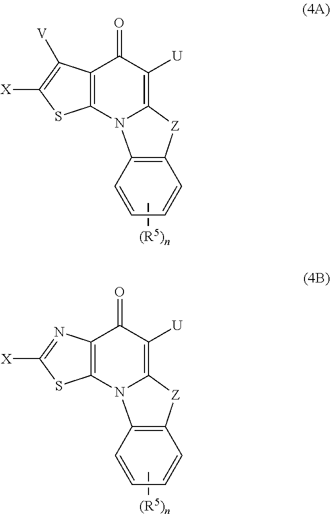

wherein U, V, X, Z, R⁵ and n are as described above.

In the above formula (1), (2A), (2B), (3), (4A) and (4B), U may be C(O)NR¹R² or SO₂NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S, where the C₃₋₆ cycloalkyl, aryl or a 5-14 membered heterocyclic ring is optionally substituted. For example, R² may be a C₁₋₁₀ alkyl substituted with an optionally substituted morpholine, thiomorpholine, imidazole, aminodithiadazole, pyrrolidine, piperazine, pyridine or piperidine ring. In other examples, R¹ and R² together with N form an optionally substituted piperidine, pyrrolidine, piperazine, morpholine, thiomorpholine, imidazole, or aminodiathiazole.

In other embodiments, U is C(O)NR¹—(CR¹₂)ₙ—NR³R⁴ or U is SO₂NR¹—(CH₂)ₙ—CHR²—NR³R⁴; where n is 1-4; and R³ and R⁴ in NR³R⁴ together form an optionally substituted piperidine, pyrrolidine, piperazine, morpholine, thiomorpholine, imidazole, or aminodiathiazole. In some examples, U is C(O)NH—(CH₂)ₙ—NR³R⁴ wherein R³ and R⁴ together with N form an optionally substituted pyrrolidine, which may be linked to (CH₂)ₙ at any position in the pyrrolidine ring. In one embodiment, R³ and R⁴ together with N from an N-methyl substituted pyrrolidine, in some embodiments, U is C(O)NH—(CH₂)₂-(1-methyl pyrrolidin-2-yl) or C(O)NH—(CH₂)₂ (2-pyrrolidin-1-yl).

In the above formula (1), (2A), (2B), (3), (4A) and (4B) Z may be S or NR². In some embodiments, at least one of V, X or Y when attached to C is halo.

In each of the above formula, each optionally substituted moiety may be substituted with acetyl, OR², amino, alkoxy, amido, halogen, cyano, an inorganic substituent; or a C₁₋₆ alkyl, C₂₋₆ alkenyl, C₂₋₆ alkynyl, —CONHR¹, each optionally substituted by halo, an oxo group, aryl or one or more heteroatoms; inorganic substituents, aryl, carbocyclic or a heterocyclic ring. Other substituents include but are not limited to alkynyl, cycloalkyl, fluorinated alkyls such as CF₃, CH₂CF₃, perfluorinated alkyls, etc.; oxygenated fluorinated alkyls such as OCF₃ or CH₂CF₃, etc.; cyano, nitro, COR², NR²COR², sulfonyl amides; NR²SOOR²; SR², SOR², COOR², CONR²₂, OCOR², OCOOR², OCONR²₂, NRCOOR², NRCONR²₂, NRC(NR)(NR²₂), NR(CO)NR²₂, and SOONR²₂, wherein each R² is as defined in formula 1.

In one embodiment, each of Z¹, Z² and Z³ are C. In another embodiment, two of Z¹, Z² and Z³ is C, and the other is N, O or S. For example, Z² and Z³ are C, and Z¹ is S. In other examples, one of Z¹, Z² and Z³ is C and the other two are N, O, or S. For example, Z² is C, Z³ is N and Z¹ is S.

In some embodiments, each of V and X is H and the corresponding Z²-Z³ are C. In other embodiments, at least one of V and X is H and the corresponding adjacent Z²-Z³ atom is C. In yet other embodiments, one of V and X is H, and the corresponding adjacent Z²-Z³ atom is C, and the other of V and X is a bond, and the corresponding adjacent Z²-Z³ atom is N.

In certain embodiments, one of V, X and Y is a halogen (e.g., fluorine) or NR², wherein R² is a C₀₋₁₀ alkyl or C₂₋₁₀ alkenyl optionally substituted with a heteroatom, a carbocyclic ring, a heterocyclic ring, an aryl or a heteroaryl; and the corresponding adjacent Z¹-Z³ is C. In yet other examples, V, Y and X independently may be selected from alkynyls, fluorinated alkyls such as CF₃, CH₂CF₃, perfluorinated alkyls, etc.; cyano, nitro, amides, sulfonyl amides, or carbonyl compounds such as COR², and the corresponding adjacent Z¹-Z³ is C. In certain embodiments, V, X and Y are H.

In each of the above formulas, U, V and X if present may contacomprise NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S; and each of the C₃₋₆ cycloalkyl, aryl or a 5-14 membered heterocyclic rings can be optionally substituted. If more than one NR¹R² moiety is present in a compound within the invention, as when both X and U comprise NR¹R² in a compound according to any one of the above formula, each R¹ and each R² is independently selected. In one example, R² is a C₁₋₁₀ alkyl substituted with an optionally substituted 5-14 membered heterocyclic ring. For example, R² may be a C₁₋₁₀ alkyl substituted with an optionally substituted morpholine, thiomorpholine, imidazole, aminodithiadazole, pyrrolidine, piperazine, pyridine or piperidine ring. Alternatively, R¹ and R² together with N may form an optionally substituted heterocyclic ring containing one or more N, O or S. For example, R¹ and R² together with N may form optionally substituted piperidine, pyrrolidine, piperazine, morpholine, thiomorpholine, imidazole, or aminodithiazole.

In one embodiment, the present invention provides compounds having formula (1), (2A) or (2B), wherein:

each of V and Y are H;

X if present is H, halo or —(R⁵)R¹R², wherein R⁵ is C or N and wherein in each —(R⁵)R¹R², R¹ and R² together may form an optionally substituted aryl or heteroaryl ring;

Z is S, NH or N-alkyl (e.g., N—CH₃);

W together with N and Z forms an optionally substituted 5- or 6-membered ring that is fused with an optionally substituted aryl or heteroaryl ring; and

U is —(C(O)C(R⁶)₂—(CH₂)ₙ—CHR²—NR³R⁴ or (C(O)NR⁶—(CH₂)ₙ—CHR²—NR³R⁴, wherein R⁶ is H or C₁₋₁₀ alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In another aspect, the present invention provides methods for preparing compounds having formula (5)

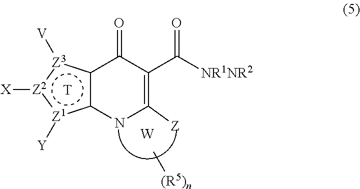

comprising contacting an ester, NHR¹R², and a Lewis acid, wherein said ester has formula (6)

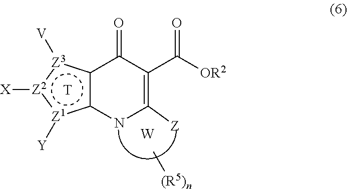

wherein T, V, W, X, Y, Z, Z¹, Z², Z³, R¹, R², R⁵ and n are as described above in formula (1);

and W together with N and Z forms an optionally substituted 5- or 6-membered ring that is fused to an optionally substituted aryl or heteroaryl, wherein said aryl or heteroaryl may be monocyclic or fused with a single or multiple ring, and wherein said ring optionally contains a heteroatom.

The present methods for preparing compounds having formula (5) may involve amide coupling of an ester with an amine in the presence of a Lewis acid such as aluminum chloride. Suitable Lewis acids may be selected by conducting a test reaction, and observing the amount of reaction product produced, as described hereafter. The present methods do not require hydrolysis of the ester to a carboxylic acid prior to amide coupling, and are simpler.

In one embodiment, the Lewis acid has formula MLₙ, wherein L is a halogen atom or an organic radical, n is 3-5, and M is a group III elemental atom, a group IV elemental atom, As, Sb, V or Fe.

In the above methods, the contacting step may be performed at room temperature. Alternatively, the ester, amine and Lewis acid may be contacted at cooler or elevated temperatures than room temperature, which may be determined by one skilled in the art.

In one embodiment, the contacting step may comprise contacting the ester and amine in an organic solvent to form a solution, and contacting the solution with a Lewis acid. In one example, the organic solvent may be methylene chloride. The reaction may also be conducted using other suitable solvents known in the art.

In another embodiment, an excess of amine in relation to the ester may be used. For example, the ratio of the ester to the amine may be 1:2; 1:1.5; or 1:1.25.

In another embodiment, an equimolar amount of Lewis acid to the amine may be used. Alternatively, the amount of Lewis acid used may be more or less than the amine.

The above methods may further comprise isolating a compound having any one of the above formula. The isolated compounds may further be purified using any methods known in the art. For example, the isolated compounds may be purified through column chromatography, recrystallization, or both.

In the above methods, the purity of the isolated compounds may be between 90 and 99%. For example, the isolated compounds may have a purity between 90 and 95%.

In the above methods, the ester may be contacted with H₂N(CR³₂)ₙ—NR³R⁴, wherein

R³ is H or C₁₋₆ alkyl;

n is 1-6; and

R⁴ is H or a C₁₋₁₀ alkyl or C₂₋₁₀ alkenyl optionally containing one or more non-adjacent heteroatoms selected from N, O and S, and optionally substituted with an optionally substituted carbocyclic or heterocyclic ring; and

wherein in NR³R⁴, R³ and R⁴ may form an optionally substituted ring such as those previously described above.

The present invention also provides pharmaceutical compositions comprising a compound having any one of the above formula, and a pharmaceutically acceptable excipient. In one example, the composition comprises a compound having any one of the above formula, polyethylene glycol, and propylene glycol in a buffer solution.

Compounds of the invention exert biological activity in assays described herein. For example, compounds of the invention can inhibit RNA biogenesis and can suppress tumor growth. Though not limiting the invention by any theory of its operation, it is believed that the compounds can function in part by interacting with quadruplex-forming regions of nucleic acids and modulating ribosomal RNA transcription. Compounds of the invention also may modulate the interaction of quadruplex-forming nucleic acids with nucleolin, a protein that is associated with apoptosis; thus modulation of the activity, localization or stability of nucleolin may also contribute to the ability of these compounds to induce apoptosis. The present invention also provides methods of preparing these compounds, and methods of using the same.

Accordingly, the present invention relates in part to methods for reducing cell proliferation and/or inducing cell death, comprising contacting a system with an effective amount of a compound having any one of the above formula, or a pharmaceutical composition thereof and optionally in combination with a chemotherapeutic agent, thereby reducing cell proliferation and/or inducing cell death, such as apoptosis or apoptotic cell death, in said system. The system may be a cell or a tissue. In one embodiment, the system includes a pancreatic cell, such as a cell from a subject or a cultured cell (e.g., in vitro or ex vivo). In particular embodiments, the system includes a pancreatic cancer cell. In one embodiment, the system is a cell line such as PC3, HCT116, HT29, MIA Paca-2, HPAC, Hs700T, Panc10.05, Panc 02.13, PL45, SW 190, Hs 766T, CFPAC-1 and PANC-1.

The present invention also provides methods for ameliorating a cell proliferative disorder, comprising administering to a subject in need thereof an effective amount of a compound having any one of the above formula, or a pharmaceutical composition thereof and optionally in combination with a chemotherapeutic agent, thereby ameliorating said cell-proliferative disorder. For example, cell proliferation may be reduced, and/or cell death, such as apoptosis or apoptotic cell death, may be induced. The cell proliferative disorder may be a tumor or a cancer in a human or animal subject. In a particular embodiment, the cancer is pancreatic cancer, including non-endocrine and endocrine tumors. Illustrative examples of non-endocrine tumors include but are not limited to adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor may be an islet cell tumor.

The above methods for reducing cell proliferation and/or inducing cell death may also be practiced in combination with a procedure and/or a chemotherapeutic agent. Examples of procedures that may be used in combination with the methods of the present invention include but are not limited to radiotherapy and surgery. In certain embodiments, the compounds of the present invention are administered in combination with a chemotherapeutic agent, and used to reduce cell proliferation, induce cell death, and/or ameliorate a cell proliferative disorder.

Furthermore, the present invention provides methods for reducing microbial titers, comprising contacting a system with an effective amount of a compound having any one of the above formula, or a pharmaceutical composition thereof and optionally with an antimicrobial agent, thereby reducing microbial titers. The system may be a cell or a tissue. The present invention also provides methods for ameliorating a microbial infection, comprising administering to a subject in need thereof an effective amount of a compound having any one of the above formula, or a pharmaceutical composition thereof and optionally with an antimicrobial agent, thereby ameliorating said microbial infection. The subject may be human or an animal. The microbial titers may be viral, bacterial or fungal titers.

The present invention also relates to methods for determining interaction selectivity between a compound having any one of the above formula, and nucleic acids capable of forming a quadruplex structure, comprising: a) contacting a compound in the absence of a competitor molecule with three or more nucleic acids capable of forming a quadruplex structure, wherein each nucleic acid is not a telomere nucleic acid; b) measuring a direct interaction between the compound and said three or more nucleic acids; and c) determining interaction selectivity from a comparison of the interaction measurements. In one example, three or more nucleic acids comprise a nucleotide sequence located 5′ of an oncogene nucleotide sequence. The oncogene may be MYC, HIF, VEGF, ABL, TGF, PDGFα, MYB, SPARC, HER, VAV, RET, H-RAS, EGF, SRC, BCL-1, BCL-2, DHFR, or HMGA. In determining interaction selectivity, the compound may be separately contacted with each of said three or more nucleic acids in a different vessel. Furthermore, the interaction selectivity may be determined from a comparison of IC₅₀ values.

The compounds of the present invention may or may not interact with regions of DNA that can form quadruplexes. In certain embodiments, the compounds of the present invention may bind and/or stabilize a propeller quadruplex. Examples of propeller quadruplexes include but are not limited to H-RAS, RET, BCL-1, DHFR, TGF-β, HIF-1α, VEGF, c-Myc, or PDGFα. In another embodiment, the compound may bind and/or stabilize a chair-eller or a basket quadruplex. For example, the compound may bind and/or stabilize BCL-2.

The present invention also provides methods for inducing cell death, such as apoptotic cell death (apoptosis), comprising administering to a system or a subject in need thereof an effective amount of a compound having any one of the above formula, or a pharmaceutical composition thereof and optionally with a chemotherapeutic agent. The present invention also provides methods for treating or ameliorating a disorder mediated by oncogene overexpression, such as c-Myc overexpression, comprising administering to a system or a subject in need thereof an effective amount of a compound having any of the formula, or a pharmaceutical composition thereof and optionally with a chemotherapeutic agent. The subject may be human or an animal, and system may be a cell or a tissue.

Compounds of the above formulas also may be capable of modulating the activities of various protein kinases, as they contain structural features that are known to bind to protein kinases, and are accordingly useful for the identification of protein kinase modulators using screening methods known in the art. Representative screening methods for certain kinases are provided herein. Accordingly, the invention provides a method for identifying a modulator of a protein kinase, which modulator sometimes is a potent modulator of one or more particular protein kinases. This method comprises screening a library of compounds described herein, which library contains at least 10 different compounds each of which is of formula 1, 2A, 2B, 3, 4A, 4B or 5, and often at least 100 of such compounds, for their ability to modulate the activity of a protein kinase. Alternatively, the method comprises screening a set of protein kinases, such as at least three or at least ten protein kinases, with a compound of formula 1, 2A, 2B, 3, 4A, 4B or 5, to determine a differential activity profile. These methods allow the user to identify a compound of formula 1, 2A, 2B, 3, 4A, 4B or 5 having a desired level of activity and/or selectivity as a protein kinase activity modulator, which compound may be used to initiate a drug development program. Thus in another embodiment, the invention provides a composition comprising an isolated protein kinase complexed with a compound of formula 1, 2A, 2B, 3, 4A, 4B or 5. Such complexes are useful for the information they provide about the binding site of a modulating compound to the particular kinase, and as a research tool for analyzing the structure of the kinase. Such complexes are also useful because they may be more readily crystallized than the uncomplexed kinase, allowing crystallization and crystal structure determination where it would not be possible without the bound modulating compound.

Also provided herein is a method for identifying a molecule that modulates an interaction between a ribosomal nucleic acid and a protein that interacts with the nucleic acid, which comprises: (a) contacting a nucleic acid containing a human ribosomal nucleotide sequence and the protein with a test molecule having any of the structures disclosed above, where the nucleic acid is capable of binding to the protein, and (b) detecting the amount of the nucleic acid bound or not bound to the protein, whereby the test molecule is identified as a molecule that modulates the interaction when a different amount of the nucleic acid binds to the protein in the presence of the test molecule than in the absence of the test molecule. In some embodiments, the protein is selected from the group consisting of Nucleolin, Fibrillarin, RecQ, QPN1 and functional fragments of the foregoing.

In some embodiments, provided is a method for identifying a molecule that causes nucleolin displacement, which comprises (a) contacting a nucleic acid containing a human ribosomal nucleotide sequence and a nucleolin protein with a test molecule of formula 1, 2A, 2B, 3, 4A, 4B or 5, where the nucleic acid is capable of binding to the nucleolin protein, and (b) detecting the amount of the nucleic acid bound or not bound to the nucleolin protein, whereby the test molecule is identified as a molecule that causes nucleolin displacement when less of the nucleic acid binds to the nucleolin protein in the presence of the test molecule than in the absence of the test molecule. In some embodiments, the nucleolin protein is in association with a detectable label, and the nucleolin protein sometimes is in association with a solid phase. The nucleic acid sometimes is in association with a detectable label, and the nucleic acid may be in association with a solid phase in certain embodiments. The nucleic acid may be DNA, RNA or an analog thereof, and may comprise a nucleotide sequence described above in specific embodiments. Provided also is a composition comprising a nucleic acid having a ribosomal nucleotide sequence provided herein, or substantially identical sequence thereof, and/or a protein that binds to the nucleotide sequence (e.g., Nucleolin, Fibrillarin, RecQ, QPN1 and functional fragments of the foregoing), and a compound of formula 1, 2A, 2B, 3, 4A, 4B or 5.

Also provided is a method for identifying a molecule that binds to a nucleic acid containing a human ribosomal nucleotide sequence, which comprises: (a) contacting a nucleic acid containing a human ribosomal nucleotide sequence described herein, a compound that binds to the nucleic acid and a test molecule of formula 1, 2A, 2B, 3, 4A, 4B or 5, and (b) detecting the amount of the compound bound or not bound to the nucleic acid, whereby the test molecule is identified as a molecule that binds to the nucleic acid when less of the compound binds to the nucleic acid in the presence of the test molecule than in the absence of the test molecule. The compound sometimes is in association with a detectable label, and at times is radiolabeled. In certain embodiments, the compound of formula 1, 2A, 2B, 3, 4A, 4B or 5 or a porphyrin. The nucleic acid may be in association with a solid phase in certain embodiments. The nucleic acid may be DNA, RNA or an analog thereof, and may comprise a nucleotide sequence described above in specific embodiments. The nucleic acid may form a quadruplex, such as an intramolecular quadruplex, in certain embodiments. Examples of ribosomal nucleotide sequences are described herein and in co-pending provisional patent application Ser. No. 60/789,109, filed Apr. 3, 2006, and entitled HUMAN RIBOSOMAL DNA (rDNA) AND RIBOSOMAL RNA (rRNA) QUADRUPLEX NUCLEIC ACIDS AND USES THEREOF. Thus, provided also is a composition comprising a compound of formula 1, 2A, 2B, 3, 4A, 4B or 5 and a nucleic acid containing a human ribosomal nucleotide sequence (e.g., a sequence from SEQ ID NO: 1, a complementary sequence thereof, or an RNA transcript of the foregoing).

Also provided herein is a method for identifying a modulator of nucleic acid synthesis, which comprises contacting a template nucleic acid, a primer oligonucleotide having a nucleotide sequence complementary to a template nucleic acid nucleotide sequence, extension nucleotides, a polymerase and a test molecule of formula 1, 2A, 2B, 3, 4A, 4B or 5, under conditions that allow the primer oligonucleotide to hybridize to the template nucleic acid, wherein the template nucleic acid comprises a human ribosomal nucleotide sequence, and detecting the presence, absence or amount of an elongated primer product synthesized by extension of the primer nucleic acid, whereby the test molecule is identified as a modulator of nucleic acid synthesis when less of the elongated primer product is synthesized in the presence of the test molecule than in the absence of the test molecule. In certain embodiments, the method is directed to identifying a modulator of RNA synthesis, and in certain embodiments, identifying a modulator of nucleolar RNA synthesis. The template nucleic acid sometimes is DNA and at times is RNA, and the template can include by way of example any one or more of the ribosomal nucleotide sequences described herein. The polymerase sometimes is a DNA polymerase and at times is a RNA polymerase. In certain embodiments, cells are contacted with a test compound of formula 1, 2A, 2B, 3, 4A, 4B or 5 and RNA levels are detected in the cells, whereby a test compound that reduces the amount of RNA compared to cells not treated with the test compound is identified as a molecule that modultes RNA synthesis. In the latter embodiments, total RNA levels may be assessed, and in some embodiments, the total amount of newly synthesized RNA may be assessed, such as by incorporation and detection of a detectable nucleotide in the RNA (e.g., radioactively labeled nucleotide (e.g., tritiated nucleotide)), for example.

In a specific assay embodiment, provided herein is a method for identifying a molecule that modulates ribosomal RNA (rRNA) synthesis, which comprises: contacting cells with a test molecule of formula 1, 2A, 2B, 3, 4A, 4B or 5, contacting a ribosomal nucleotide sequence with one or more primers that amplify a portion thereof and a labeled probe that hybridizes to the amplification product, and detecting the amount of the amplification product by hybridization of the labeled probe, whereby a test molecule that reduces or increases the amount of amplification product is identified as a molecule that modulates rRNA synthesis. The labeled probe in some embodiments is added after the primers are added and the rRNA is amplified, and in certain embodiments, the labeled probe and the primers are added at the same time. The portion of ribosomal nucleotide sequence amplified sometimes is at the 5′ end of rDNA.

In certain embodiments, the invention provides a library of compounds, which library comprises at least 10 compounds of formula 1, 2A, 2B, 3, 4A, 4B or 5. The library preferably contains at least 100 such compounds. This library can be used to identify compounds having one or more of the activities described herein, or a specific combination of such activities using methods known in the art. The method is particularly useful for identifying molecules having a threshold level of biological activity, including but not limited to (a) binding to quadruplex nucleic acid or inhibiting formation of quadruplex nucleic acid (rDNA or rRNA), (b) activity against a specific protein kinase or set of protein kinases and (c) activity as a modulator of binding of a nucleic acid to a protein, such as nucleolin, for example.

### BRIEF DESCRIPTION OF THE DRAWINGS

FIG. 1 shows the effect of an exemplary compound of the present invention on RNA synthesis in an HCT-116 colorectal cancer xenograft model.

FIG. 2 shows the pharmacokinetics of an exemplary compound of the present invention.

FIGS. 3A and 3B show the activity of an exemplary compound of the present invention in an HCT-116 colorectal cancer xenograft model.

### DEFINITIONS

As used herein, the term “alkyl” refers to a carbon-containing compound, and encompasses compounds containing one or more heteroatoms. The term “alkyl” also encompasses alkyls substituted with one or more substituents including but not limited to OR¹, amino, amido, halo, ═O, aryl, heterocyclic groups, or inorganic substituents.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems.

As used herein, the term “aryl” refers to a polyunsaturated, typically aromatic hydrocarbon substituent, whereas a “heteroaryl” or “heteroaromatic” refer to an aromatic ring containing a heteroatom. The aryl and heteroaryl structures encompass compounds having monocyclic, bicyclic or multiple ring systems.

As used herein, the term “heteroatom” refers to any atom that is not carbon or hydrogen, such as nitrogen, oxygen or sulfur.

Illustrative examples of heterocycles include but are not limited to tetrahydrofuran, 1,3-dioxolane, 2,3-dihydrofuran, pyran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, imidazolidine-2,4-dione, 1,3-dihydrobenzimidazol-2-one, indole, thiazole, benzothiazole, thiadiazole, thiophene, tetrahydro-thiophene 1,1-dioxide, diazepine, triazole, guanidine, diazabicyclo[2.2.1]heptane, 2,5-diazabicyclo[2.2.1]heptane, 2,3,4,4a,9,9a-hexahydro-1H-β-carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, sulfonyls, sulfinyls, phosphates, etc.

The terms “treat,” “treatment” and “therapeutic effect” as used herein refer to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganism include but are not limited to virus, bacterium and fungus.

As used herein, the term “chemotherapeutic agent” refers to a therapeutic agent that may be used for treating or ameliorating a cell proliferative disorder such as tumors or cancer. Examples of chemotherapeutic agents include but are not limited to an antineoplastic agent, an alkylating agent, a plant alkaloid, an antimicrobial agent, a sulfonamide, an antiviral agent, a platinum agent, and other anticancer agents known in the art. Particular examples of chemotherapeutic agents include but are not limited to cisplatin, carboplatin, busulphan, methotrexate, daunorubicin, doxorubicin, cyclophosphamide, mephalan, vincristine, vinblastine, chlorambucil, paclitaxel, gemcitabine, and others known in the art. (See e.g., Goodman & Gilman's, The Pharmacological Basis of Therapeutics (9th Ed) (Goodman, et al., eds.) (McGraw-Hill) (1996); and 1999 Physician's Desk Reference (1998)).

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

### DESCRIPTION OF THE INVENTION

The present invention relates to pyridinone compounds having formula (1), (2A), (2B), (3), (4A), (4B) and (5), and pharmaceutically acceptable salts, esters, and prodrugs thereof. The present invention also relates to methods for using the compounds described herein, such as in screening and in treatment. The compounds of the present invention may or may not interact with regions of DNA that can form quadruplexes.

The compounds of present invention having formula (1), (2A), (2B), (3), (4A), (4B) and (5) are reproduced below:

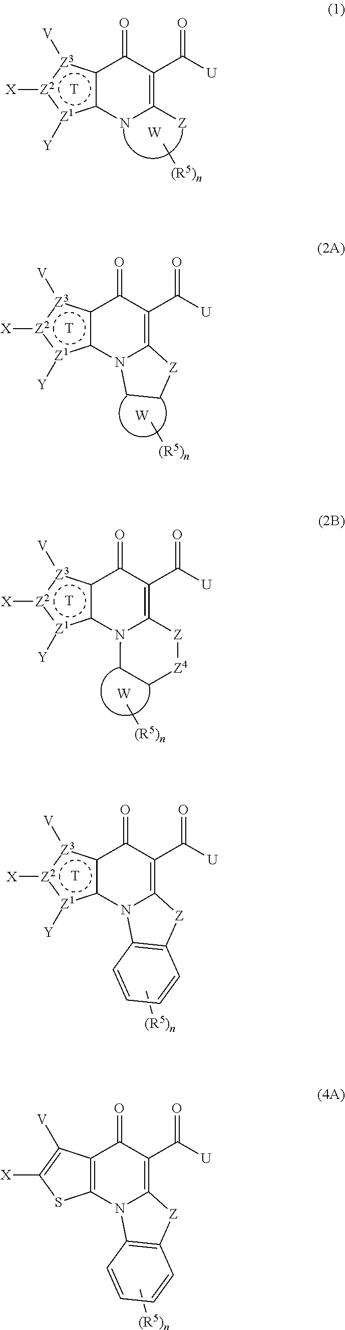

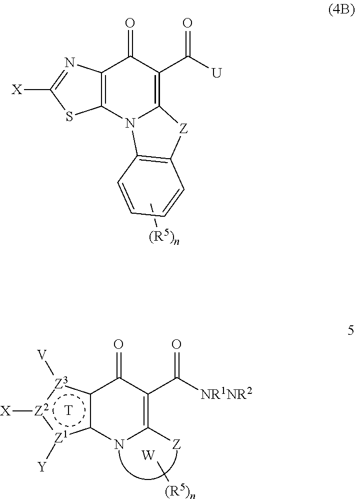

wherein T, U, V, W, X, Y, Z, Z¹, Z², Z³, R⁵ and n are as described above.

The compounds of the present invention may be chiral. As used herein, a chiral compound is a compound that is different from its mirror image, and has an enantiomer. Furthermore, the compounds may be racemic, or an isolated enantiomer or stereoisomer. Methods of synthesizing chiral compounds and resolving a racemic mixture of enantiomers are well known to those skilled in the art. See, e.g., March, “Advanced Organic Chemistry,” John Wiley and Sons, Inc., New York, (1985), which is incorporated herein by reference.

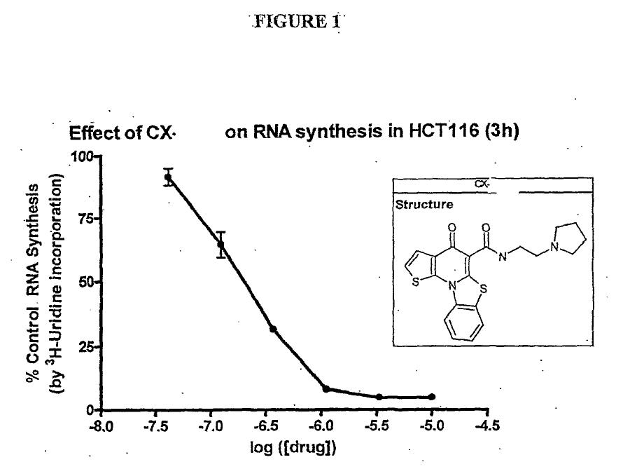

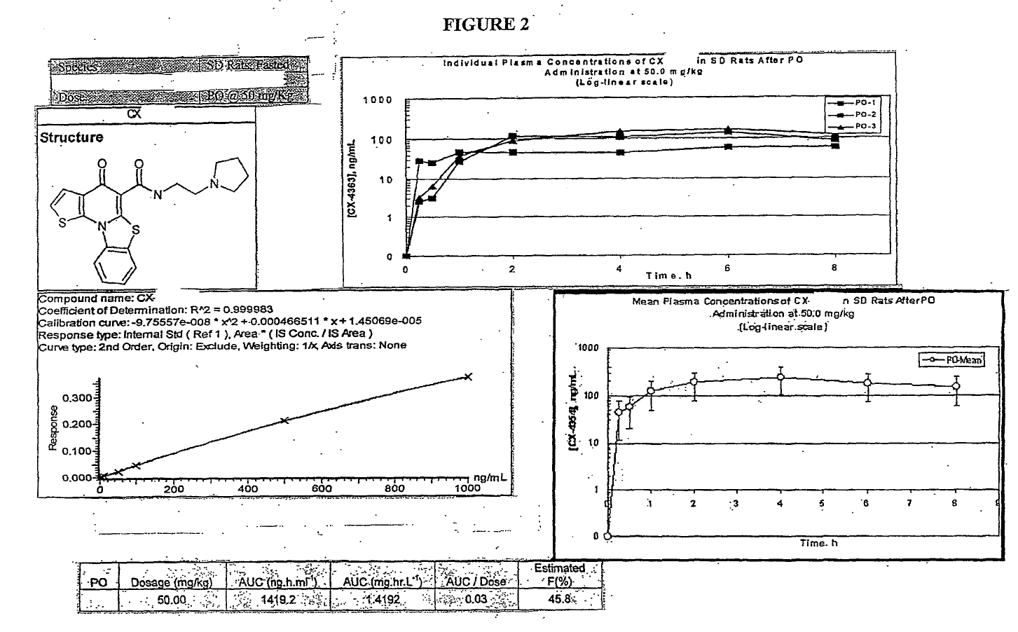

Exemplary compounds of the present invention were tested using screening assays such as those described herein. FIGS. 1-3 show the activity of an exemplary compound of the present invention in an HCT-116 colorectal cancer xenograft model.

The present invention also encompasses other compounds having any one formula (1), (2A), (2B), (3), (4A), (4B) and (5), comprising substituents U, V, X and Y independently selected from the substituents exemplified in the Examples. Thus, the present invention is not limited to the specific combination of substituents described in various embodiments below.

The compounds described herein may interact with regions of nucleic acids that can form quadruplexes. Because regions of DNA that can form quadruplexes are regulators of biological processes such as oncogene transcription, modulators of quadruplex biological activity can be utilized as cancer therapeutics. Molecules that interact with regions of DNA that can form quadruplexes can exert a therapeutic effect on certain cell proliferative disorders and related conditions. Particularly, abnormally increased oncogene expression can cause cell proliferative disorders, and quadruplex structures typically down-regulate oncogene expression. Examples of oncogenes include but are not limited to MYC, HIF, VEGF, ABL, TGF, PDGFA, MYB, SPARC, HUMTEL, HER, VAV, RET, H-RAS, EGF, SRC, BCL1, BCL2, DHFR, HMGA, and other oncogenes known to one of skill in the art. Furthermore, the compounds described herein may induce cell death (e.g., apoptosis) and not interact with regions of DNA that can form quadruplexes.

Molecules that bind to regions of DNA that can form quadruplexes can exert a biological effect according to different mechanisms, which include for example, stabilizing a native quadruplex structure, inhibiting conversion of a native quadruplex to duplex DNA by blocking strand cleavage, and stabilizing a native quadruplex structure having a quadruplex-destabilizing nucleotide substitution and other sequence specific interactions. Thus, compounds that bind to regions of DNA that can form quadruplexes described herein may be administered to cells, tissues, or organisms for the purpose of down-regulating oncogene transcription and thereby treating cell proliferative disorders.

Determining whether the biological activity of native DNA that can form quadruplexes is modulated in a cell, tissue, or organism can be accomplished by monitoring quadruplex biological activity. Quadruplex forming regions of DNA biological activity may be monitored in cells, tissues, or organisms, for example, by detecting a decrease or increase of gene transcription in response to contacting the quadruplex forming DNA with a molecule. Transcription can be detected by directly observing RNA transcripts or observing polypeptides translated by transcripts, which are methods well known in the art.

Molecules that interact with quadruplex forming DNA and quadruplex forming nucleic acids can be utilized to treat many cell proliferative disorders. Cell proliferative disorders include, for example, colorectal cancers and hematopoietic neoplastic disorders (i.e., diseases involving hyperplastic/neoplastic cells of hematopoietic origin such as those arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (Vaickus, Crit. Rev. in Oncol./Hemotol. 11:267-297 (1991)). Lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. Cell proliferative disorders also include cancers of the colorectum, breast, lung, liver, pancreas, lymph node, colon, prostate, brain, head and neck, skin, liver, kidney, and heart. Compounds that interact with regions of DNA that may form quadruplexes also can be utilized to target cancer related processes and conditions, such as increased angiogenesis, by inhibiting angiogenesis in a subject.

The present invention provides a method for reducing cell proliferation or for treating or alleviating cell proliferative disorders, comprising contacting a system having a native DNA capable of forming a quadruplex region with a compound having any one of the above formula. The system may be a group of cells or one or more tissues. In one embodiment, the system is a subject in need of a treatment of a cell proliferative disorder (e.g., a mammal such as a mouse, rat, monkey, or human). The present invention also provides a method for treating colorectal cancer by administering a compound that interacts with a c-MYC quadruplex forming region to a subject in need thereof, thereby reducing the colorectal cancer cell proliferation. Furthermore, the present invention provides a method for inhibiting angiogenesis and optionally treating a cancer associated with angiogenesis, comprising administering a compound that interacts with a vascular endothelial growth factor (VEGF) quadruplex forming region to a subject in need thereof, thereby reducing angiogenesis and optionally treating a cancer associated with angiogenesis.

Compounds that interact with quadruplex forming regions of DNA can also be used to reduce a microbial infection, such as a viral infection. Retroviruses offer a wealth of potential targets for G-quadruplex targeted therapeutics. G-quadruplex structures have been implicated as functional elements in at least two secondary structures formed by either viral RNA or DNA in HIV, the dimer linker structure (DLS) and the central DNA flap (CDF). Additionally, DNA aptamers which are able to adopt either inter- or intramolecular quadruplex structures are able to inhibit viral replication. In one example, DNA aptamers are able to inhibit viral replication by targeting the envelope glycoprotein (putatively). In another example, DNA aptamers inhibit viral replication by targeting the HIV-integrase respectively, suggesting the involvement of native quadruplex structures in interaction with the integrase enzyme.

Dimer linker structures, which are common to all retroviruses, serve to bind two copies of the viral genome together by a non-covalent interaction between the two 5′ ends of the two viral RNA sequences. The genomic dimer is stably associated with the gag protein in the mature virus particle. In the case of HIV, the origin of this non-covalent binding may be traced to a 98 base-pair sequence containing several runs of at least two consecutive guanines (e.g., the 3′ for the formation of RNA dimers in vitro). An observed cation (potassium) dependence for the formation and stability of the dimer in vitro, in addition to the failure of an antisense sequence to effectively dimerize, has revealed the most likely binding structure to be an intermolecular G-quadruplex.

Prior to integration into the host genome, reverse transcribed viral DNA forms a pre-integration complex (PIC) with at least two major viral proteins, integrase and reverse transcriptase, which is subsequently transported into the nucleus. The Central DNA Flap (CDF) refers to 99-base length single-stranded tail of the +strand, occurring near the center of the viral duplex DNA, which is known to a play a role in the nuclear import of the PIC. Oligonucleotide mimics of the CDF have been shown to form intermolecular G-quadruplex structures in cell-free systems.

Thus, compounds that recognize quadruplex forming regions can be used to stabilize the dimer linker structure and thus prevent de-coupling of the two RNA strands. Also, by binding to the quadruplex structure formed by the CDF, protein recognition and/or binding events for nuclear transport of the PIC may be disrupted. In either case, a substantial advantage can exist over other anti-viral therapeutics. Current Highly Active Anti-Retroviral Therapeutic (HAART) regimes rely on the use of combinations of drugs targeted towards the HIV protease and HIV integrase. The requirement for multi-drug regimes is to minimize the emergence of resistance, which will usually develop rapidly when agents are used in isolation. The source of such rapid resistance is the infidelity of the reverse transcriptase enzyme which makes a mutation approximately once in every 10,000 base pairs. An advantage of targeting viral quadruplex structures over protein targets, is that the development of resistance is slow or is impossible. A point mutation of the target quadruplex can compromise the integrity of the quadruplex structure and lead to a non-functional copy of the virus. A single therapeutic agent based on this concept may replace the multiple drug regimes currently employed, with the concomitant benefits of reduced costs and the elimination of harmful drug/drug interactions.

The present invention provides a method for reducing a microbial titer in a system, comprising contacting a system having a native DNA quadruplex forming region with a compound having any one of the above formula. The system may be one or more cells or tissues. Examples of microbial titers include but are not limited to viral, bacterial or fungal titers. In a particular embodiment, the system is a subject in need of a treatment for a viral infection (e.g., a mammal such as a mouse, rat, monkey, or human). Examples of viral infections include infections by a hepatitis virus (e.g., hepatitis B or C), human immunodeficiency virus (HIV), rhinovirus, herpes-zoster virus (VZV), herpes simplex virus (e.g., HSV-1 or HSV-2), cytomegalovirus (CMV), vaccinia virus, influenza virus, encephalitis virus, hantavirus, arbovirus, West Nile virus, human papilloma virus (HPV), Epstein-Barr virus, and respiratory syncytial virus. The present invention also provides a method for treating HIV infection by administering a compound having any one of the above formula to a subject in need thereof, thereby reducing the HIV infection.

Identifying Compounds that can Bind to Quadruplex Forming Regions of DNA

Compounds described herein may bind to quadruplex forming regions of DNA where a biological activity of this region, often expressed as a “signal,” produced in a system containing the compound is different than the signal produced in a system not containing the compound. While background signals may be assessed each time a new molecule is probed by the assay, detecting the background signal is not required each time a new molecule is assayed.

Examples of quadruplex forming nucleotide sequences are set forth in the following Table 2:

| | SEQ | |
|---|---|---|
| | ID | |
| SEQUENCE | NO | ORIGIN |
| TG₄AG₃TG₄AG₃TG₄AAGG | 1 | CMYC |
| GGGGGGGGGGGGGCGGGGGCGGGGGCGGGGGAGGGGC | 2 | PDGFA |
| G₈ACGCG₃AGCTG₅AG₃CTTG₄CCAG₃CG₄CGCTTAG₅ | 3 | PDGF |
| | | B/c-sis |
| AGGAAGGGGAGGGCCGGGGGGAGGTGGC | 4 | CABL |
| AGGGGCGGGGCGGGGCGGGGGC | 5 | RET |
| AG₄CG₃CGCGGGAGGAAGGGGGCGGGAGCGGGGCTG | 6 | BCL-2 |
| GGGGGGCGGGGGCGGGCGCAGGGGGAGGGGGC | 7 | Cyclin |
| | | D1/BCL-1 |
| CGGGGCGGGGCGGGGGCGGGGGC | 8 | H-RAS |
| AGAGGAGGAGGAGGTCACGGAGGAGGAGGAGAAGGAG | 9 | CMYB |
| GAGGAGGAA or AGAGGAGGAGGAGGACACGGAGGAG | | |
| GAGGAGAAGGAGGAGGAGGAA | | |
| (GGA)₄ | 10 | VAV |
| AGAGAAGAGGGGAGGAGGAGGAGGAGAGGAGGAGGCGC | 11 | HMGA2 |
| GGAGGGGGAGGGG | 12 | CPIM |
| AGGAGAAGGAGGAGGTGGAGGAGGAGG | 13 | HER2/ |
| | | neu |
| AGGAGGAGGAGAATGCGAGGAGGAGGGAGGAGA | 14 | EGFR |
| GGGGCGGGCCGGGGGCGGGGTCCCGGCGGGGCGGAG | 15 | VEGF |
| CGGGAGGAGGAGGAAGGAGGAAGCGCG | 16 | CSRC |

In addition to determining whether a test molecule or test nucleic acid gives rise to a different signal, the affinity of the interaction between the nucleic acid and the compound may be quantified. IC₅₀, K_{d}, or Kᵢ threshold values may be compared to the measured IC₅₀ or K_{d} values for each interaction, and thereby identify a test molecule as a quadruplex interacting molecule or a test nucleic acid as a quadruplex forming nucleic acid. For example, IC₅₀ or K_{d} threshold values of 10 μM or less, 1 μM or less, and 100 nM or less are often utilized. In another example, threshold values of 10 nM or less, 1 nM or less, 100 pM or less, and 10 pM or less may be utilized to identify quadruplex interacting molecules and quadruplex forming nucleic acids.

Many assays are available for identifying compounds that have affinity for quadruplex forming regions of DNA. In some of these assays, the biological activity is the quadruplex nucleic acid binding to a compound and binding is measured as a signal. In other assays, the biological activity is a polymerase arresting function of a quadruplex and the degree of arrest is measured as a decrease in a signal. In certain assays, the biological activity is transcription and transcription levels can be quantified as a signal. In another assay, the biological activity is cell death and the number of cells undergoing cell death is quantified. Another assay monitors proliferation rates of cancer cells. Examples of assays are fluorescence binding assays, gel mobility shift assays (see, e.g., Jin & Pike, Mol. Endocrinol. (1996) 10:196-205), polymerase arrest assays, transcription reporter assays, cancer cell proliferation assays, and apoptosis assays (see, e.g., Amersham Biosciences (Piscataway, N.J.)), and embodiments of such assays are described hereafter in Example 8. Also, topoisomerase assays can be utilized to determine whether the quadruplex interacting molecules have a topoisomerase pathway activity (see, e.g., TopoGEN, Inc. (Columbus, Ohio)).

### Formulation of Compounds

As used herein, the term “pharmaceutically acceptable salts, esters and amides” includes but are not limited to carboxylate salts, amino acid addition salts, esters and amides of the compounds, as well as the zwitterionic forms thereof, which are known to those skilled in the art as suitable for use with humans and animals. (See, e.g., Gerge, S. M., et al., “Pharmaceutical Salts,” J. Pharm. Sci. (1977) 66:1-19, which is incorporated herein by reference.)

Any suitable formulation of the compounds described herein can be prepared. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art. For example, pharmaceutically acceptable salts may be obtained by reacting a sufficiently basic compound such as an amine with a suitable acid affording a physiologically acceptable anion. Alkali metal (e.g., sodium, potassium or lithium) or alkaline earth metal (e.g., calcium) salts of carboxylic acids also are made.

A compound may be formulated as a pharmaceutical composition and administered to a mammalian host in need of such treatment. In one embodiment, the mammalian host is human. Any suitable route of administration may be used, including but not limited to oral, parenteral, intravenous, intramuscular, topical and subcutaneous routes.

In one embodiment, a compound is administered systemically (e.g., orally) in combination with a pharmaceutically acceptable vehicle such as an inert diluent or an assimilable edible carrier. They may be enclosed in hard or soft shell gelatin capsules, compressed into tablets, or incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may be varied and may conveniently be between about 2 to about 60% of the weight of a given unit dosage form. The amount of active compound in such therapeutically useful compositions is such that an effective dosage level will be obtained.

Tablets, troches, pills, capsules, and the like also may contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form is pharmaceutically acceptable and substantially non-toxic in the amounts employed. In addition, the active compound may be incorporated into sustained-release preparations and devices.

The active compound also may be administered intravenously or intraperitoneally by infusion or injection. Solutions of the active compound or its salts may be prepared in a buffered solution, often phosphate buffered saline, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The compound is sometimes prepared as a polymatrix-containing formulation for such administration (e.g., a liposome or microsome). Liposomes are described for example in U.S. Pat. No. 5,703,055 (Feigner, et al.) and Gregoriadis, Liposome Technology vols. I to III (2nd ed. 1993).

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in liquid form. Compounds often are administered as compositions or formulations, in combination with a dermatologically acceptable carrier, which may be a solid or a liquid. Examples of useful dermatological compositions used to deliver compounds to the skin are known (see, e.g., Jacquet, et al. (U.S. Pat. No. 4,608,392), Geria (U.S. Pat. No. 4,992,478), Smith, et al. (U.S. Pat. No. 4,559,157) and Wortzman (U.S. Pat. No. 4,820,508).

Compounds may be formulated with a solid carrier, which include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Generally, the concentration of the compound in a liquid composition often is from about 0.1 wt % to about 25 wt %, sometimes from about 0.5 wt % to about 10 wt %. The concentration in a semi-solid or solid composition such as a gel or a powder often is about 0.1 wt % to about 5 wt %, sometimes about 0.5 wt % to about 2.5 wt %. A compound composition may be prepared as a unit dosage form, which is prepared according to conventional techniques known in the pharmaceutical industry. In general terms, such techniques include bringing a compound into association with pharmaceutical carrier(s) and/or excipient(s) in liquid form or finely divided solid form, or both, and then shaping the product if required.

Table 3 shows examples of formulations which may be used with compounds described herein. For example, a compound may be formulated having dosages from 10 mg/mL to 20 mg/mL solution, using the formulations herein. In Table 3, the designation “D5W” refers to deionized water with 5% dextrose. Each component in each formulation may be varied without affecting the activity of the compound. In one example, the compound is formulated in a solution comprising polyethylene glycol and propylene glycol in a buffer solution such as a phosphate buffer.

TABLE 3

| | | | | | pH of the |
|---|---|---|---|---|---|
| | | | Compound (mL) + | pH of the | formulated |
| | | % | Placebo | Placebo | solution |
| | Formulations | (w/w) | solution (mL) | solution | (10 mg/mL) |
| 1. | Mannitol | 4 | 35 ml + 35 mL | 6.1 | 6.1 |
| | Sucrose | 0.5 | | | |
| | 5% D5W solution | 95.5 | | | |
| 2. | Mannitol | 4 | 35 ml + 35 mL | 6 | 5.8 |
| | 50 mM PO₄ buffer, pH = 6.0 | 96 | | | |
| 3. | Mannitol | 4 | 35 ml + 35 mL | 5 | 5 |
| | 50 mM Citrate buffer, pH = 5.0 | 96 | | | |
| 4. | Mannitol | 4 | 35 ml + 35 mL | 6 | 6 |
| | 5% D5W | 96 | | | |
| 5. | Test compound (20 mg/mL) | 1 | 35 ml + 35 mL | 6.4 | 6.1 |
| | 5% D5W | 99 | | | |
| 6. | PEG 300 | 7 | 5 ml + 5 mL | N/A | 5.80 |
| | Propylene glycol | 9 | | | |
| | 5% D5W | 84 | | | |
| 7. | PEG 300 | 7 | 5 ml + 5 mL | N/A | 5.8 |
| | Propylene glycol | 9 | | | |
| | 50 mM PO₄ buffer, pH = 6.0 | 84 | | | |
| 8. | Mannitol | 4 | 5 ml + 5 mL | N/A | 5.7 |
| | PEG 300 | 20 | | | |
| | 50 mM PO₄ buffer, pH = 6.0 | 76 | | | |
| 9. | Mannitol | 4 | 5 ml + 5 mL | N/A | 5.8 |
| | Propylene glycol | 10 | | | |
| | 50 mM PO₄ buffer, pH = 6.0 | 86 | | | |

The compound composition may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

Dosages

A useful compound dosage often is determined by assessing its in vitro activity in a cell or tissue system and/or in vivo activity in an animal system. For example, methods for extrapolating an effective dosage in mice and other animals to humans are known to the art (see, e.g., U.S. Pat. No. 4,938,949). Such systems can be used for determining the LD₅₀ (the dose lethal to 50% of the population) and the ED₅₀ (the dose therapeutically effective in 50% of the population) of a compound. The dose ratio between a toxic and therapeutic effect is the therapeutic index and it can be expressed as the ratio ED₅₀/LD₅₀. The compound dosage often lies within a range of circulating concentrations for which the ED₅₀ is associated with little or no toxicity. The dosage may vary within this range depending upon the dosage form employed and the route of administration utilized. For any compounds used in the methods described herein, the therapeutically effective dose can be estimated initially from cell culture assays. A dose sometimes is formulated to achieve a circulating plasma concentration range covering the IC₅₀ (i.e., the concentration of the test compound which achieves a half-maximal inhibition of symptoms) as determined in in vitro assays, as such information often is used to more accurately determine useful doses in humans. Levels in plasma may be measured, for example, by high performance liquid chromatography.

Another example of effective dose determination for a subject is the ability to directly assay levels of “free” and “bound” compound in the serum of the test subject. Such assays may utilize antibody mimics and/or “biosensors” generated by molecular imprinting techniques. The compound is used as a template, or “imprinting molecule”, to spatially organize polymerizable monomers prior to their polymerization with catalytic reagents. Subsequent removal of the imprinted molecule leaves a polymer matrix which contains a repeated “negative image” of the compound and is able to selectively rebind the molecule under biological assay conditions (see, e.g., Ansell, et al., Current Opinion in Biotechnology (1996) 7:89-94 and in Shea, Trends in Polymer Science (1994) 2:166-173).

Such “imprinted” affinity matrixes are amenable to ligand-binding assays, whereby the immobilized monoclonal antibody component is replaced by an appropriately imprinted matrix (see, e.g., Vlatakis, et al., Nature (1993) 361:645-647). Through the use of isotope-labeling, “free” concentration of compound can be readily monitored and used in calculations of IC₅₀. Such “imprinted” affinity matrixes can also be designed to include fluorescent groups whose photon-emitting properties measurably change upon local and selective binding of compound. These changes can be readily assayed in real time using appropriate fiberoptic devices, in turn allowing the dose in a test subject to be quickly optimized based on its individual IC₅₀. An example of such a “biosensor” is discussed in Kriz, et al., Analytical Chemistry (1995) 67:2142-2144.

Exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The following examples are offered to illustrate but not to limit the invention.

### Example 1

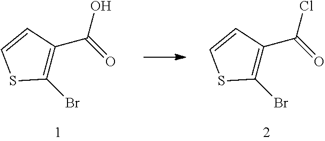

2-bromo-3-thiophene carboxylic acid 1 (1.0 eq, 5.30 g, 25.59 mmol) was suspended in CH₂Cl₂ (80 ml). Oxalylchloride (1.1 eq, 2.5 ml, 28.46 mmol) and 5 drops of DMF were added. The reaction was stirred overnight at room temperature and the volatiles removed in vacuo to afford a brown solid (5.58 g, 97% yield).

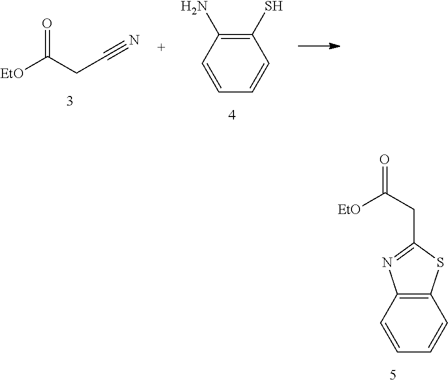

2-aminothiophenol 4 (1.0 eq, 20.0 g, 160 mmol) and ethyl cyanoacetate 5 (4.0 eq, 60 ml, 639 mmol) were stirred at 120° overnight. The reaction mixture was poured on silica gel and eluted with a 1:9 mixture of AcOEt and hexanes. The material was purified a second time by flash chromatography on silica gel (2.5 to 10% gradient of AcOEt in hexanes) to afford a yellow viscous oil (22.6 g, 65% yield). LCMS (ES): 95% pure, m/z 222 [M+1]⁺.

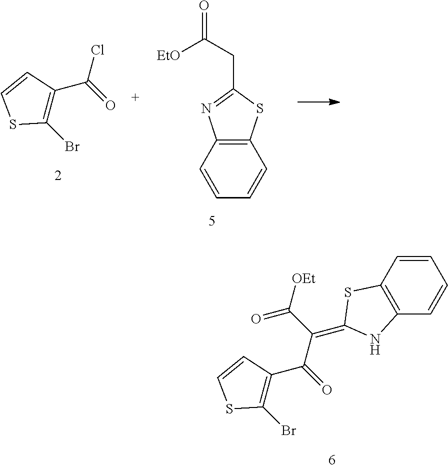

Under nitrogen atmosphere, compound 5 (1.0 eq, 2.37 g, 10.71 mmol) was dissolved in acetonitrile (20 ml). MgCl₂ (1.5 eq, 1.53 g, 16.07 mmol) was added and the resulting suspension was stirred at room temperature for a few minutes and then cooled with water-ice bath. A solution of 2 (1.0 eq, 2.42 g, 10.73 mmol) in acetonitrile (20 ml) was added dropwise and the solution stirred at 0° C. for 5 min Triethylamine was added dropwise through syringe (2.0 eq, 3.0 ml, 21.52 mmol) while maintaining the internal temperature below 10° C. After the addition was complete, the cooling bath was removed and the reaction mixture was stirred at room temperature for 19 hours. The volatiles were removed in vacuo. CH₂Cl₂ and a 1N aqueous HCl solution were added and the biphasic mixture stirred for a few minutes. The two phases were separated and an extra CH₂Cl₂ extraction was carried out. The combined organic extracts were washed with brine, dried over Na₂SO₄ and the solvent removed. Trituration in AcOEt and filtration provided compound 6 as a white powder (3.43 g, 78% yield). LCMS (ES): 95% pure, m/z 410 [M]⁺, 412 [M+2]⁺.

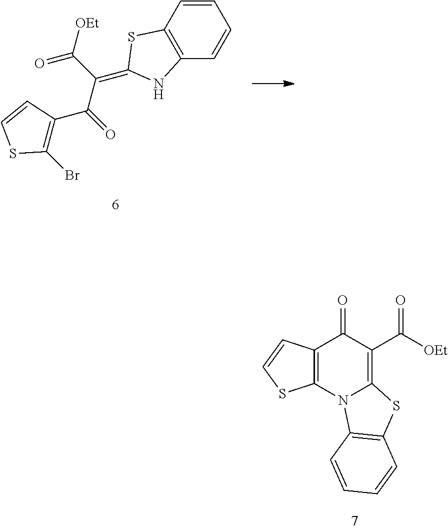

Compound 6 (1.0 eq., 2.53 g, 6.17 mmol) was dissolved in anhydrous NMP (15 ml). K₂CO₃ (3.5 eq, 2.98 g, 21.55 mmol) was added and the suspension vigorously stirred at 150° C. for 5 hours. The mixture was cooled down and the precipitate formed during the reaction was filtered and washed with NMP. The material was stirred in water for a few minutes and filtered. After drying, trituration in hot AcOEt, filtration and drying, compound 7 was isolated as a gray solid (1.20 g, 59% yield). LCMS (ES): 95% pure, m/z 330 [M+1]⁺.

### Example 2

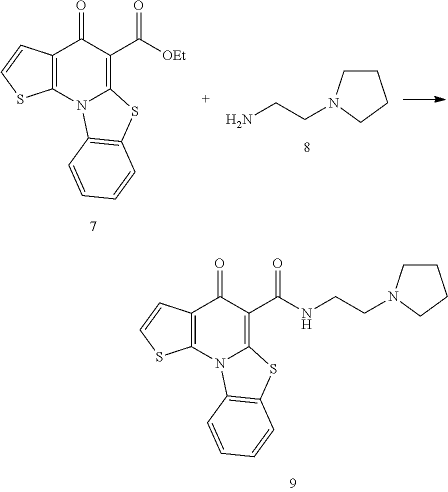

Compound 7 (1.0 eq, 2.098 g, 6.369 mmol), compound 8 (2.0 eq, 1.61 ml, 12.70 mmol) and DBU (4.0 eq, 3.8 ml, 25.40 mmol) were mixed together in CH₂Cl₂ (50 ml). AlCl₃ (2.0 eq, 1.7 g, 12.75 mmol) was added and the mixture stirred at 45° C. for 2 hours. After removal of CH₂Cl₂ in vacuo, the resulting slurry was treated with a saturated aqueous tartaric acid solution (ca. 20 ml) and stirred for an hour at room temperature. Water was added and the pH was adjusted to 14 by adding NaOH. The mixture was poured in a funnel and shacked with an equal volume of CH₂Cl₂. The resulting emulsion was filtered through a fitted glass to remove any solid material. After separation of the organic phase, the aqueous phase was further extracted with CH₂Cl₂. The combined extracts were washed with brine, dried over Na₂SO₄ and the solvents removed in vacuo. The material was purified by flash chromatography on silica gel (5 to 20% gradient of MeOH in CH₂Cl₂). Trituration in Et₂O and filtration provided 9 as an off white solid (793 mg, 32% yield). LCMS (ES): 99% pure, m/z 398 [M+1]⁺.

Illustrative examples of the compounds of the present invention are shown in table 1 (attached document).

TABLE 1

| | | LCMS(ES) | HCT-116 |
|---|---|---|---|
| Structure | M.W. | m/z | MTS IC₅₀ (uM) |
| 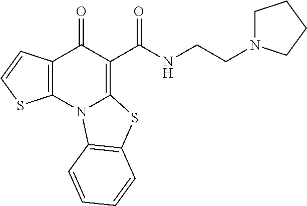 | 397.51 | 398 [M + 1]+ | 0.04 |
| 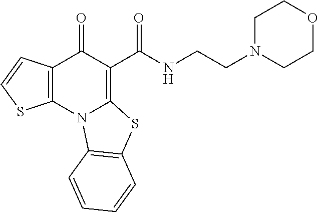 | 413.51 | 414 [M + 1]+ | 2.1 |
| 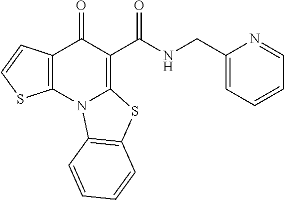 | 391.47 | 392 [M + 1]+ | 2.05 |
| 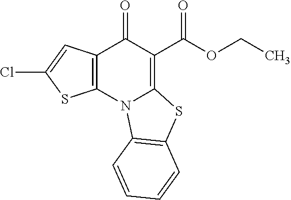 | 363.84 | 364 [M + 1]+ | 10 |
| 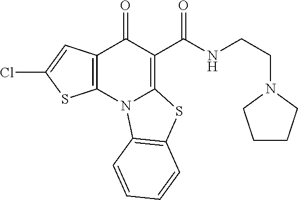 | 431.96 | 432 [M + 1]+ | 0.01 |
| 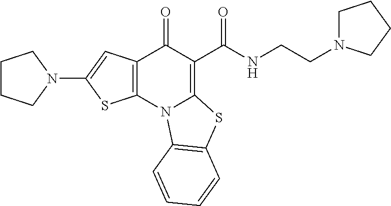 | 466.62 | 467 [M + 1]+ | 0.05 |
| 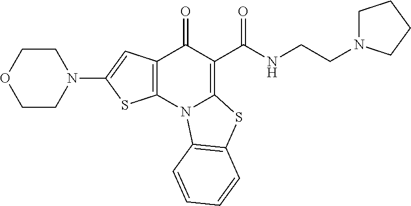 | 482.62 | 483 [M + 1]+ | |
| 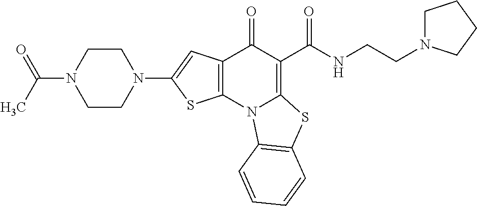 | 523.67 | 524 [M + 1]+ | |
| 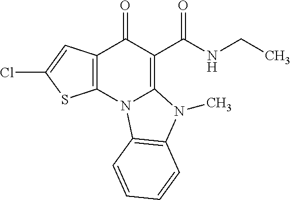 | 360.81 | 361 [M + 1]+ | |
| 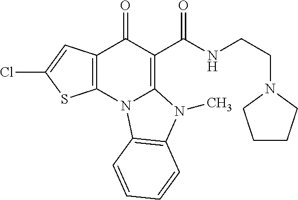 | 428.94 | 429 [M + 1]+ | |
| 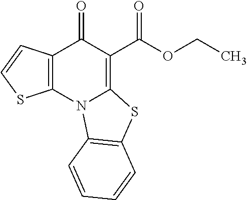 | 329.39 | 330 [M + 1]+ | 9.8 |
| 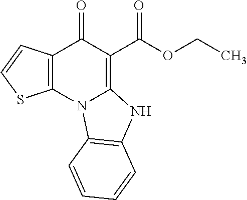 | 312.34 | 313 [M + 1]+ | 10 |
| 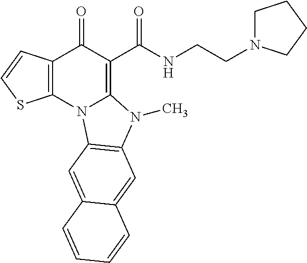 | 444.55 | 445 [M + 1]+ | 0.3 |
| 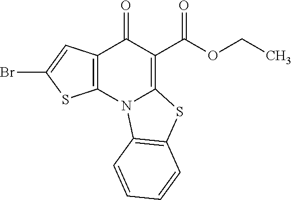 | 408.29 | 408 [M]+, 410 [M + 2]+ | 10 |
| 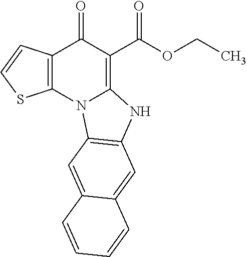 | 362.40 | 363 [M + 1]+ | 4 |
| 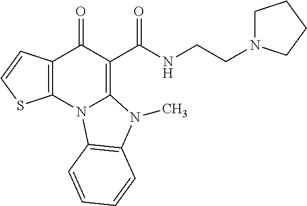 | 394.49 | 395 [M + 1]+ | 3.2 |
| 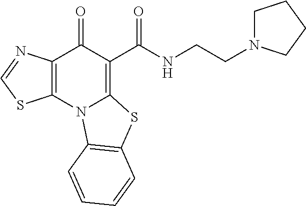 | 398.50 | 399 [M + 1]+ | |

### Example 3

### Quadruplex Structures of Ribosomal Nucleic Acids

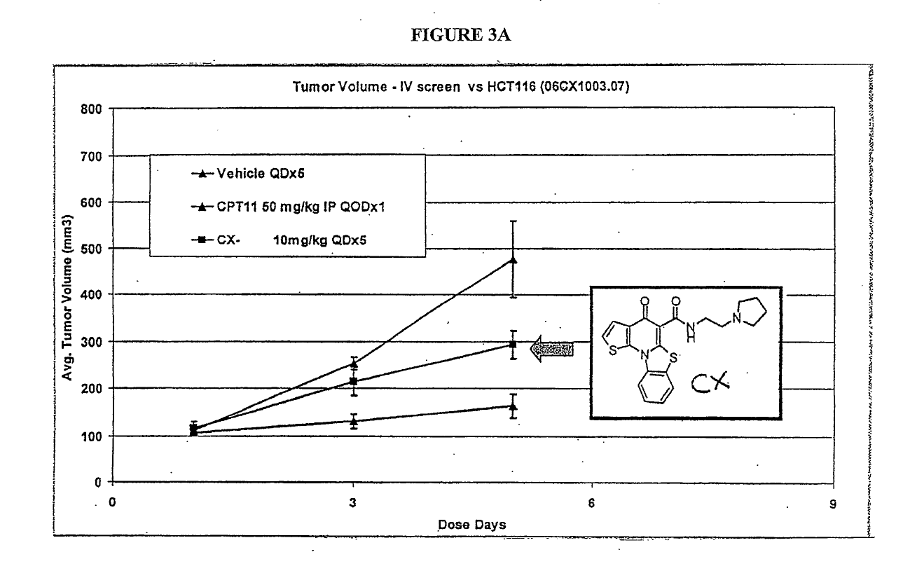

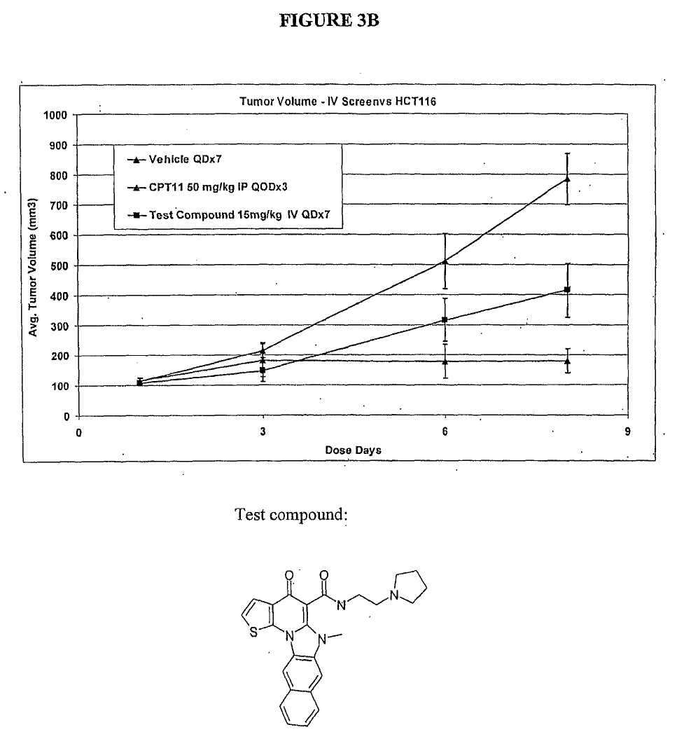

Circular dichroism (CD) was utilized to determine whether subsequences from ribosomal nucleic acids form quadruplex structures. All sequences were HPLC purified DNA oligonucleotides (sequences 5′ to 3′ as represented hereafter). The name of each sample in FIGS. 3A and 3B identifies the approximate location along the rDNA unit as well as the specific strand (NC=non-coding; C=coding). The following procedure was utilized: each oligonucleotide was dissolved at a strand concentration of 5 uM in 200 ul of aqueous buffer containing Tris pH 7.4 (10 mM). The sample was heated to 95° C. for 5 min. then allowed to cool to ambient temperature. CD spectroscopy was performed on a JASCO 810 Spectropolarimeter, using a quartz cell of 1 mm path length. Additional spectra were taken after the addition of 20 ul KCl (1M) to the oligonucleotide solution. Compound A-1 has been shown to interact preferentially with a mixed-parallel quadruplex structure in competition assays (e.g., PCT/US2004/033401 filed on Oct. 7, 2004, entitled “Competition Assay for Identifying Modulators of Quadruplex Nucleic Acids”). The nucleotide sequence of a representative human rDNA sequence (SEQ ID NO: 1) is provided hereafter:

| | |
|---|---|
| 1 | gctgacacgc tgtcctctgg cgacctgtcg tcggagaggt tgggcctccg gatgcgcgcg |
| 61 | gggctctggc ctcacggtga ccggctagcc ggccgcgctc ctgccttgag ccgcctgccg |
| 121 | cggcccgcgg gcctgctgtt ctctcgcgcg tccgagcgtc ccgactcccg gtgccggccc |
| 181 | gggtccgggt ctctgaccca cccgggggcg gcggggaagg cggcgagggc caccgtgccc |
| 241 | cgtgcgctct ccgctgcggg cgcccggggc gccgcacaac cccacccgct ggctccgtgc |
| 301 | cgtgcgtgtc aggcgttctc gtctccgcgg ggttgtccgc cgccccttcc ccggagtggg |
| 361 | gggtggccgg agccgatcgg ctcgctggcc ggccggcctc cgctcccggg gggctcttcg |
| 421 | atcgatgtgg tgacgtcgtg ctctcccggg ccgggtccga gccgcgacgg gcgaggggcg |
| 481 | gacgttcgtg gcgaacggga ccgtccttct cgctccgccc gcgcggtccc ctcgtctgct |
| 541 | cctctccccg cccgccggcc ggcgtgtggg aaggcgtggg gtgcggaccc cggcccgacc |
| 601 | tcgccgtccc gcccgccgcc ttcgcttcgc gggtgcgggc cggcggggtc ctctgacgcg |
| 661 | gcagacagcc ctgcctgtcg cctccagtgg ttgtcgactt gcgggcggcc cccctccgcg |
| 721 | gcggtggggg tgccgtcccg ccggcccgtc gtgctgccct ctcggggggg gtttgcgcga |
| 781 | gcgtcggctc cgcctgggcc cttgcggtgc tcctggagcg ctccgggttg tccctcaggt |
| 841 | gcccgaggcc gaacggtggt gtgtcgttcc cgcccccggc gccccctcct ccggtcgccg |
| 901 | ccgcggtgtc cgcgcgtggg tcctgaggga gctcgtcggt gtggggttcg aggcggtttg |
| 961 | agtgagacga gacgagacgc gcccctccca cgcggggaag ggcgcccgcc tgctctcggt |
| 1021 | gagcgcacgt cccgtgctcc cctctggcgg gtgcgcgcgg gccgtgtgag cgatcgcggt |
| 1081 | gggttcgggc cggtgtgacg cgtgcgccgg ccggccgccg aggggctgcc gttctgcctc |
| 1141 | cgaccggtcg tgtgtgggtt gacttcggag gcgctctgcc tcggaaggaa ggaggtgggt |
| 1201 | ggacgggggg gcctggtggg gttgcgcgca cgcgcgcacc ggccgggccc ccgccctgaa |
| 1261 | cgcgaacgct cgaggtggcc gcgcgcaggt gtttcctcgt accgcagggc cccctccctt |
| 1321 | ccccaggcgt ccctcggcgc ctctgcgggc ccgaggagga gcggctggcg ggtgggggga |
| 1381 | gtgtgaccca ccctcggtga gaaaagcctt ctctagcgat ctgagaggcg tgccttgggg |
| 1441 | gtaccggatc ccccgggccg ccgcctctgt ctctgcctcc gttatggtag cgctgccgta |
| 1501 | gcgacccgct cgcagaggac cctcctccgc ttccccctcg acggggttgg gggggagaag |
| 1561 | cgagggttcc gccggccacc gcggtggtgg ccgagtgcgg ctcgtcgcct actgtggccc |
| 1621 | gcgcctcccc cttccgagtc gggggaggat cccgccgggc cgggcccggc gctcccaccc |
| 1681 | agcgggttgg gacgcggcgg ccggcgggcg gtgggtgtgc gcgcccggcg ctctgtccgg |
| 1741 | cgcgtgaccc cctccgtccg cgagtcggct ctccgcccgc tcccgtgccg agtcgtgacc |
| 1801 | ggtgccgacg accgcgtttg cgtggcacgg ggtcgggccc gcctggccct gggaaagcgt |
| 1861 | cccacggtgg gggcgcgccg gtctcccgga gcgggaccgg gtcggaggat ggacgagaat |
| 1921 | cacgagcgac ggtggtggtg gcgtgtcggg ttcgtggctg cggtcgctcc ggggcccccg |
| 1981 | gtggcggggc cccggggctc gcgaggcggt tctcggtggg ggccgagggc cgtccggcgt |
| 2041 | cccaggcggg gcgccgcggg accgccctcg tgtctgtggc ggtgggatcc cgcggccgtg |
| 2101 | ttttcctggt ggcccggccg tgcctgaggt ttctccccga gccgccgcct ctgcgggctc |
| 2161 | ccgggtgccc ttgccctcgc ggtccccggc cctcgcccgt ctgtgccctc ttccccgccc |
| 2221 | gccgcccgcc gatcctcttc ttccccccga gcggctcacc ggcttcacgt ccgttggtgg |
| 2281 | ccccgcctgg gaccgaaccc ggcaccgcct cgtggggcgc cgccgccggc cactgatcgg |
| 2341 | cccggcgtcc gcgtcccccg gcgcgcgcct tggggaccgg gtcggtggcg cgccgcgtgg |
| 2401 | ggcccggtgg gcttcccgga gggttccggg ggtcggcctg cggcgcgtgc gggggaggag |
| 2461 | acggttccgg gggaccggcc gcggctgcgg cggcggcggt ggtgggggga gccgcgggga |
| 2521 | tcgccgaggg ccggtcggcc gccccgggtg ccccgcggtg ccgccggcgg cggtgaggcc |
| 2581 | ccgcgcgtgt gtcccggctg cggtcggccg cgctcgaggg gtccccgtgg cgtccccttc |
| 2641 | cccgccggcc gcctttctcg cgccttcccc gtcgccccgg cctcgcccgt ggtctctcgt |
| 2701 | cttctcccgg cccgctcttc cgaaccgggt cggcgcgtcc cccgggtgcg cctcgcttcc |
| 2761 | cgggcctgcc gcggcccttc cccgaggcgt ccgtcccggg cgtcggcgtc ggggagagcc |
| 2821 | cgtcctcccc gcgtggcgtc gccccgttcg gcgcgcgcgt gcgcccgagc gcggcccggt |
| 2881 | ggtccctccc ggacaggcgt tcgtgcgacg tgtggcgtgg gtcgacctcc gccttgccgg |
| 2941 | tcgctcgccc tctccccggg tcggggggtg gggcccgggc cggggcctcg gccccggtcg |
| 3001 | ctgcctcccg tcccgggcgg gggcgggcgc gccggccggc ctcggtcgcc ctcccttggc |
| 3061 | cgtcgtgtgg cgtgtgccac ccctgcgccg gcgcccgccg gcggggctcg gagccgggct |
| 3121 | tcggccgggc cccgggccct cgaccggacc ggctgcgcgg gcgctgcggc cgcacggcgc |
| 3181 | gactgtcccc gggccgggca ccgcggtccg cctctcgctc gccgcccgga cgtcggggcc |
| 3241 | gccccgcggg gcgggcggag cgccgtcccc gcctcgccgc cgcccgcggg cgccggccgc |
| 3301 | gcgcgcgcgc gcgtggccgc cggtccctcc cggccgccgg gcgcgggtcg ggccgtccgc |
| 3361 | ctcctcgcgg gcgggcgcga cgaagaagcg tcgcgggtct gtggcgcggg gcccccggtg |
| 3421 | gtcgtgtcgc gtggggggcg ggtggttggg gcgtccggtt cgccgcgccc cgccccggcc |
| 3481 | ccaccggtcc cggccgccgc ccccgcgccc gctcgctccc tcccgtccgc ccgtccgcgg |
| 3541 | cccgtccgtc cgtccgtccg tcgtcctcct cgcttgcggg gcgccgggcc cgtcctcgcg |
| 3601 | aggccccccg gccggccgtc cggccgcgtc gggggctcgc cgcgctctac cttacctacc |
| 3661 | tggttgatcc tgccagtagc atatgcttgt ctcaaagatt aagccatgca tgtctaagta |
| 3721 | cgcacggccg gtacagtgaa actgcgaatg gctcattaaa tcagttatgg ttcctttggt |
| 3781 | cgctcgctcc tctcctactt ggataactgt ggtaattcta gagctaatac atgccgacgg |
| 3841 | gcgctgaccc ccttcgcggg ggggatgcgt gcatttatca gatcaaaacc aacccggtca |
| 3901 | gcccctctcc ggccccggcc ggggggcggg cgccggcggc tttggtgact ctagataacc |
| 3961 | tcgggccgat cgcacgcccc ccgtggcggc gacgacccat tcgaacgtct gccctatcaa |
| 4021 | ctttcgatgg tagtcgccgt gcctaccatg gtgaccacgg gtgacgggga atcagggttc |
| 4081 | gattccggag agggagcctg agaaacggct accacatcca aggaaggcag caggcgcgca |
| 4141 | aattacccac tcccgacccg gggaggtagt gacgaaaaat aacaatacag gactctttcg |
| 4201 | aggccctgta attggaatga gtccacttta aatcctttaa cgaggatcca ttggagggca |
| 4261 | agtctggtgc cagcagccgc ggtaattcca gctccaatag cgtatattaa agttgctgca |
| 4321 | gttaaaaagc tcgtagttgg atcttgggag cgggcgggcg gtccgccgcg aggcgagcca |
| 4381 | ccgcccgtcc ccgccccttg cctctcggcg ccccctcgat gctcttagct gagtgtcccg |
| 4441 | cggggcccga agcgtttact ttgaaaaaat tagagtgttc aaagcaggcc cgagccgcct |
| 4501 | ggataccgca gctaggaata atggaatagg accgcggttc tattttgttg gttttcggaa |
| 4561 | ctgaggccat gattaagagg gacggccggg ggcattcgta ttgcgccgct agaggtgaaa |
| 4621 | ttcttggacc ggcgcaagac ggaccagagc gaaagcattt gccaagaatg ttttcattaa |
| 4681 | tcaagaacga aagtcggagg ttcgaagacg atcagatacc gtcgtagttc cgaccataaa |
| 4741 | cgatgccgac cggcgatgcg gcggcgttat tcccatgacc cgccgggcag cttccgggaa |
| 4801 | accaaagtct ttgggttccg gggggagtat ggttgcaaag ctgaaactta aaggaattga |
| 4861 | cggaagggca ccaccaggag tggagcctgc ggcttaattt gactcaacac gggaaacctc |
| 4921 | acccggcccg gacacggaca ggattgacag attgatagct ctttctcgat tccgtgggtg |
| 4981 | gtggtgcatg gccgttctta gttggtggag cgatttgtct ggttaattcc gataacgaac |
| 5041 | gagactctgg catgctaact agttacgcga cccccgagcg gtcggcgtcc cccaacttct |
| 5101 | tagagggaca agtggcgttc agccacccga gattgagcaa taacaggtct gtgatgccct |
| 5161 | tagatgtccg gggctgcacg cgcgctacac tgactggctc agcgtgtgcc taccctacgc |
| 5221 | cggcaggcgc gggtaacccg ttgaacccca ttcgtgatgg ggatcgggga ttgcaattat |
| 5281 | tccccatgaa cgagggaatt cccgagtaag tgcgggtcat aagcttgcgt tgattaagtc |
| 5341 | cctgcccttt gtacacaccg cccgtcgcta ctaccgattg gatggtttag tgaggccctc |
| 5401 | ggatcggccc cgccggggtc ggcccacggc cctggcggag cgctgagaag acggtcgaac |
| 5461 | ttgactatct agaggaagta aaagtcgtaa caaggtttcc gtaggtgaac ctgcggaagg |
| 5521 | atcattaacg gagcccggag ggcgaggccc gcggcggcgc cgccgccgcc gcgcgcttcc |
| 5581 | ctccgcacac ccaccccccc accgcgacgc ggcgcgtgcg cgggcggggc ccgcgtgccc |
| 5641 | gttcgttcgc tcgctcgttc gttcgccgcc cggccccgcc gccgcgagag ccgagaactc |
| 5701 | gggagggaga cgggggggag agagagagag agagagagag agagagagag agagagagaa |
| 5761 | agaagggcgt gtcgttggtg tgcgcgtgtc gtggggccgg cgggcggcgg ggagcggtcc |
| 5821 | ccggccgcgg ccccgacgac gtgggtgtcg gcgggcgcgg gggcggttct cggcggcgtc |
| 5881 | gcggcgggtc tgggggggtc tcggtgccct cctccccgcc ggggcccgtc gtccggcccc |
| 5941 | gccgcgccgg ctccccgtct tcggggccgg ccggattccc gtcgcctccg ccgcgccgct |
| 6001 | ccgcgccgcc gggcacggcc ccgctcgctc tccccggcct tcccgctagg gcgtctcgag |
| 6061 | ggtcgggggc cggacgccgg tcccctcccc cgcctcctcg tccgcccccc cgccgtccag |
| 6121 | gtacctagcg cgttccggcg cggaggttta aagacccctt ggggggatcg cccgtccgcc |
| 6181 | cgtgggtcgg gggcggtggt gggcccgcgg gggagtcccg tcgggagggg cccggcccct |
| 6241 | cccgcgcctc caccgcggac tccgctcccc ggccggggcc gcgccgccgc cgccgccgcg |
| 6301 | gcggccgtcg ggtgggggct ttacccggcg gccgtcgcgc gcctgccgcg cgtgtggcgt |
| 6361 | gcgccccgcg ccgtgggggc gggaaccccc gggcgcctgt ggggtggtgt ccgcgctcgc |
| 6421 | ccccgcgtgg gcggcgcgcg cctccccgtg gtgtgaaacc ttccgacccc tctccggagt |
| 6481 | ccggtcccgt ttgctgtctc gtctggccgg cctgaggcaa ccccctctcc tcttgggcgg |
| 6541 | ggggggcggg gggacgtgcc gcgccaggaa gggcctcctc ccggtgcgtc gtcgggagcg |
| 6601 | ccctcgccaa atcgacctcg tacgactctt agcggtggat cactcggctc gtgcgtcgat |
| 6661 | gaagaacgca gctagctgcg agaattaatg tgaattgcag gacacattga tcatcgacac |
| 6721 | ttcgaacgca cttgcggccc cgggttcctc ccggggctac gcctgtctga gcgtcgcttg |
| 6781 | ccgatcaatc gccccggggg tgcctccggg ctcctcgggg tgcgcggctg ggggttccct |
| 6841 | cgcagggccc gccgggggcc ctccgtcccc ctaagcgcag acccggcggc gtccgccctc |
| 6901 | ctcttgccgc cgcgcccgcc ccttccccct ccccccgcgg gccctgcgtg gtcacgcgtc |
| 6961 | gggtggcggg ggggagaggg gggcgcgccc ggctgagaga gacggggagg gcggcgccgc |
| 7021 | cgccggaaga cggagaggga aagagagagc cggctcgggc cgagttcccg tggccgccgc |
| 7081 | ctgcggtccg ggttcctccc tcggggggct ccctcgcgcc gcgcgcggct cggggttcgg |
| 7141 | ggttcgtcgg ccccggccgg gtggaaggtc ccgtgcccgt cgtcgtcgtc gtcgcgcgtc |
| 7201 | gtcggcggtg ggggcgtgtt gcgtgcggtg tggtggtggg ggaggaggaa ggcgggtccg |
| 7261 | gaaggggaag ggtgccggcg gggagagagg gtcgggggag cgcgtcccgg tcgccgcggt |
| 7321 | tccgccgccc gcccccggtg gcggcccggc gtccggccga ccggccgctc cccgcgcccc |
| 7381 | tcctcctccc cgccgcccct cctccgaggc cccgcccgtc ctcctcgccc tccccgcgcg |
| 7441 | tacgcgcgcg cgcccgcccg cccggctcgc ctcgcggcgc gtcggccggg gccgggagcc |
| 7501 | cgccccgccg cccgcccgtg gccgcggcgc cggggttcgc gtgtccccgg cggcgacccg |
| 7561 | cgggacgccg cggtgtcgtc cgccgtcgcg cgcccgcctc cggctcgcgg ccgcgccgcg |
| 7621 | ccgcgccggg gccccgtccc gagcttccgc gtcggggcgg cgcggctccg ccgccgcgtc |
| 7681 | ctcggacccg tccccccgac ctccgcgggg gagacgcgcc ggggcgtgcg gcgcccgtcc |
| 7741 | cgcccccggc ccgtgcccct ccctccggtc gtcccgctcc ggcggggcgg cgcgggggcg |
| 7801 | ccgtcggccg cgcgctctct ctcccgtcgc ctctccccct cgccgggccc gtctcccgac |
| 7861 | ggagcgtcgg gcgggcggtc gggccggcgc gattccgtcc gtccgtccgc cgagcggccc |
| 7921 | gtccccctcc gagacgcgac ctcagatcag acgtggcgac ccgctgaatt taagcatatt |
| 7981 | agtcagcgga ggaaaagaaa ctaaccagga ttccctcagt aacggcgagt gaacagggaa |
| 8041 | gagcccagcg ccgaatcccc gccccgcggg gcgcgggaca tgtggcgtac ggaagacccg |
| 8101 | ctccccggcg ccgctcgtgg ggggcccaag tccttctgat cgaggcccag cccgtggacg |
| 8161 | gtgtgaggcc ggtagcggcc ggcgcgcgcc cgggtcttcc cggagtcggg ttgcttggga |
| 8221 | atgcagccca aagcgggtgg taaactccat ctaaggctaa ataccggcac gagaccgata |
| 8281 | gtcaacaagt accgtaaggg aaagttgaaa agaactttga agagagagtt caagagggcg |
| 8341 | tgaaaccgtt aagaggtaaa cgggtggggt ccgcgcagtc cgcccggagg attcaacccg |
| 8401 | gcggcgggtc cggccgtgtc ggcggcccgg cggatctttc ccgccccccg ttcctcccga |
| 8461 | cccctccacc cgccctccct tcccccgccg cccctcctcc tcctccccgg agggggcggg |
| 8521 | ctccggcggg tgcgggggtg ggcgggcggg gccgggggtg gggtcggcgg gggaccgtcc |
| 8581 | cccgaccggc gaccggccgc cgccgggcgc atttccaccg cggcggtgcg ccgcgaccgg |
| 8641 | ctccgggacg gctgggaagg cccggcgggg aaggtggctc ggggggcccc gtccgtccgt |
| 8701 | ccgtcctcct cctcccccgt ctccgccccc cggccccgcg tcctccctcg ggagggcgcg |
| 8761 | cgggtcgggg cggcggcggc ggcggcggtg gcggcggcgg cgggggcggc gggaccgaaa |
| 8821 | ccccccccga gtgttacagc ccccccggca gcagcactcg ccgaatcccg gggccgaggg |
| 8881 | agcgagaccc gtcgccgcgc tctcccccct cccggcgccc acccccgcgg ggaatccccc |
| 8941 | gcgagggggg tctcccccgc gggggcgcgc cggcgtctcc tcgtgggggg gccgggccac |
| 9001 | ccctcccacg gcgcgaccgc tctcccaccc ctcctccccg cgcccccgcc ccggcgacgg |
| 9061 | ggggggtgcc gcgcgcgggt cggggggcgg ggcggactgt ccccagtgcg ccccgggcgg |
| 9121 | gtcgcgccgt cgggcccggg ggaggttctc tcggggccac gcgcgcgtcc cccgaagagg |
| 9181 | gggacggcgg agcgagcgca cggggtcggc ggcgacgtcg gctacccacc cgacccgtct |
| 9241 | tgaaacacgg accaaggagt ctaacacgtg cgcgagtcgg gggctcgcac gaaagccgcc |
| 9301 | gtggcgcaat gaaggtgaag gccggcgcgc tcgccggccg aggtgggatc ccgaggcctc |
| 9361 | tccagtccgc cgagggcgca ccaccggccc gtctcgcccg ccgcgccggg gaggtggagc |
| 9421 | acgagcgcac gtgttaggac ccgaaagatg gtgaactatg cctgggcagg gcgaagccag |
| 9481 | aggaaactct ggtggaggtc cgtagcggtc ctgacgtgca aatcggtcgt ccgacctggg |
| 9541 | tataggggcg aaagactaat cgaaccatct agtagctggt tccctccgaa gtttccctca |
| 9601 | ggatagctgg cgctctcgca gacccgacgc acccccgcca cgcagtttta tccggtaaag |
| 9661 | cgaatgatta gaggtcttgg ggccgaaacg atctcaacct attctcaaac tttaaatggg |
| 9721 | taagaagccc ggctcgctgg cgtggagccg ggcgtggaat gcgagtgcct agtgggccac |
| 9781 | ttttggtaag cagaactggc gctgcgggat gaaccgaacg ccgggttaag gcgcccgatg |
| 9841 | ccgacgctca tcagacccca gaaaaggtgt tggttgatat agacagcagg acggtggcca |
| 9901 | tggaagtcgg aatccgctaa ggagtgtgta acaactcacc tgccgaatca actagccctg |
| 9961 | aaaatggatg gcgctggagc gtcgggccca tacccggccg tcgccggcag tcgagagtgg |
| 10021 | acgggagcgg cgggggcggc gcgcgcgcgc gcgcgtgtgg tgtgcgtcgg agggcggcgg |
| 10081 | cggcggcggc ggcgggggtg tggggtcctt cccccgcccc cccccccacg cctcctcccc |
| 10141 | tcctcccgcc cacgccccgc tccccgcccc cggagccccg cggacgctac gccgcgacga |
| 10201 | gtaggagggc cgctgcggtg agccttgaag cctagggcgc gggcccgggt ggagccgccg |
| 10261 | caggtgcaga tcttggtggt agtagcaaat attcaaacga gaactttgaa ggccgaagtg |
| 10321 | gagaagggtt ccatgtgaac agcagttgaa catgggtcag tcggtcctga gagatgggcg |
| 10381 | agcgccgttc cgaagggacg ggcgatggcc tccgttgccc tcggccgatc gaaagggagt |
| 10441 | cgggttcaga tccccgaatc cggagtggcg gagatgggcg ccgcgaggcg tccagtgcgg |
| 10501 | taacgcgacc gatcccggag aagccggcgg gagccccggg gagagttctc ttttctttgt |
| 10561 | gaagggcagg gcgccctgga atgggttcgc cccgagagag gggcccgtgc cttggaaagc |
| 10621 | gtcgcggttc cggcggcgtc cggtgagctc tcgctggccc ttgaaaatcc gggggagagg |
| 10681 | gtgtaaatct cgcgccgggc cgtacccata tccgcagcag gtctccaagg tgaacagcct |
| 10741 | ctggcatgtt ggaacaatgt aggtaaggga agtcggcaag ccggatccgt aacttcggga |
| 10801 | taaggattgg ctctaagggc tgggtcggtc gggctggggc gcgaagcggg gctgggcgcg |
| 10861 | cgccgcggct ggacgaggcg cgcgcccccc ccacgcccgg ggcacccccc tcgcggccct |
| 10921 | cccccgcccc acccgcgcgc gccgctcgct ccctccccac cccgcgccct ctctctctct |
| 10981 | ctctcccccg ctccccgtcc tcccccctcc ccgggggagc gccgcgtggg ggcgcggcgg |
| 11041 | ggggagaagg gtcggggcgg caggggccgc gcggcggccg ccggggcggc cggcgggggc |
| 11101 | aggtccccgc gaggggggcc ccggggaccc ggggggccgg cggcggcgcg gactctggac |
| 11161 | gcgagccggg cccttcccgt ggatcgcccc agctgcggcg ggcgtcgcgg ccgcccccgg |
| 11221 | ggagcccggc ggcggcgcgg cgcgcccccc acccccaccc cacgtctcgg tcgcgcgcgc |
| 11281 | gtccgctggg ggcgggagcg gtcgggcggc ggcggtcggc gggcggcggg gcggggcggt |
| 11341 | tcgtcccccc gccctacccc cccggccccg tccgcccccc gttcccccct cctcctcggc |
| 11401 | gcgcggcggc ggcggcggca ggcggcggag gggccgcggg ccggtccccc ccgccgggtc |
| 11461 | cgcccccggg gccgcggttc cgcgcgcgcc tcgcctcggc cggcgcctag cagccgactt |
| 11521 | agaactggtg cggaccaggg gaatccgact gtttaattaa aacaaagcat cgcgaaggcc |
| 11581 | cgcggcgggt gttgacgcga tgtgatttct gcccagtgct ctgaatgtca aagtgaagaa |
| 11641 | attcaatgaa gcgcgggtaa acggcgggag taactatgac tctcttaagg tagccaaatg |
| 11701 | cctcgtcatc taattagtga cgcgcatgaa tggatgaacg agattcccac tgtccctacc |
| 11761 | tactatccag cgaaaccaca gccaagggaa cgggcttggc ggaatcagcg gggaaagaag |
| 11821 | accctgttga gcttgactct agtctggcac ggtgaagaga catgagaggt gtagaataag |
| 11881 | tgggaggccc ccggcgcccc cccggtgtcc ccgcgagggg cccggggcgg ggtccgcggc |
| 11941 | cctgcgggcc gccggtgaaa taccactact ctgatcgttt tttcactgac ccggtgaggc |
| 12001 | gggggggcga gcccgagggg ctctcgcttc tggcgccaag cgcccgcccg gccgggcgcg |
| 12061 | acccgctccg gggacagtgc caggtgggga gtttgactgg ggcggtacac ctgtcaaacg |
| 12121 | gtaacgcagg tgtcctaagg cgagctcagg gaggacagaa acctcccgtg gagcagaagg |
| 12181 | gcaaaagctc gcttgatctt gattttcagt acgaatacag accgtgaaag cggggcctca |
| 12241 | cgatccttct gaccttttgg gttttaagca ggaggtgtca gaaaagttac cacagggata |
| 12301 | actggcttgt ggcggccaag cgttcatagc gacgtcgctt tttgatcctt cgatgtcggc |
| 12361 | tcttcctatc attgtgaagc agaattcgcc aagcgttgga ttgttcaccc actaataggg |
| 12421 | aacgtgagct gggtttagac cgtcgtgaga caggttagtt ttaccctact gatgatgtgt |
| 12481 | tgttgccatg gtaatcctgc tcagtacgag aggaaccgca ggttcagaca tttggtgtat |
| 12541 | gtgcttggct gaggagccaa tggggcgaag ctaccatctg tgggattatg actgaacgcc |
| 12601 | tctaagtcag aatcccgccc aggcgaacga tacggcagcg ccgcggagcc tcggttggcc |
| 12661 | tcggatagcc ggtcccccgc ctgtccccgc cggcgggccg cccccccctc cacgcgcccc |
| 12721 | gccgcgggag ggcgcgtgcc ccgccgcgcg ccgggaccgg ggtccggtgc ggagtgccct |
| 12781 | tcgtcctggg aaacggggcg cggccggaaa ggcggccgcc ccctcgcccg tcacgcaccg |
| 12841 | cacgttcgtg gggaacctgg cgctaaacca ttcgtagacg acctgcttct gggtcggggt |
| 12901 | ttcgtacgta gcagagcagc tccctcgctg cgatctattg aaagtcagcc ctcgacacaa |
| 12961 | gggtttgtcc gcgcgcgcgt gcgtgcgggg ggcccggcgg gcgtgcgcgt tcggcgccgt |
| 13021 | ccgtccttcc gttcgtcttc ctccctcccg gcctctcccg ccgaccgcgg cgtggtggtg |
| 13081 | gggtgggggg gagggcgcgc gaccccggtc ggccgccccg cttcttcggt tcccgcctcc |
| 13141 | tccccgttca cgccggggcg gctcgtccgc tccgggccgg gacggggtcc ggggagcgtg |
| 13201 | gtttgggagc cgcggaggcg ccgcgccgag ccgggccccg tggcccgccg gtccccgtcc |
| 13261 | cgggggttgg ccgcgcggcg cggtgggggg ccacccgggg tcccggccct cgcgcgtcct |
| 13321 | tcctcctcgc tcctccgcac gggtcgaccg acgaaccgcg ggtggcgggc ggcgggcggc |
| 13381 | gagccccacg ggcgtccccg cacccggccg acctccgctc gcgacctctc ctcggtcggg |
| 13441 | cctccggggt cgaccgcctg cgcccgcggg cgtgagactc agcggcgtct cgccgtgtcc |
| 13501 | cgggtcgacc gcggccttct ccaccgagcg gcggtgtagg agtgcccgtc gggacgaacc |
| 13561 | gcaaccggag cgtccccgtc tcggtcggca cctccggggt cgaccagctg ccgcccgcga |
| 13621 | gctccggact tagccggcgt ctgcacgtgt cccgggtcga ccagcaggcg gccgccggac |
| 13681 | gcagcggcgc acgcacgcga gggcgtcgat tccccttcgc gcgcccgcgc ctccaccggc |
| 13741 | ctcggcccgc ggtggagctg ggaccacgcg gaactccctc tcccacattt ttttcagccc |
| 13801 | caccgcgagt ttgcgtccgc gggaccttta agagggagtc actgctgccg tcagccagta |
| 13861 | ctgcctcctc ctttttcgct tttaggtttt gcttgccttt tttttttttt tttttttttt |
| 13921 | ttttttcttt ctttctttct ttctttcttt ctttctttct ttctttcttt cgcttgtctt |
| 13981 | cttcttgtgt tctcttcttg ctcttcctct gtctgtctct ctctctctct ctctctctgt |
| 14041 | ctctcgctct cgccctctct ctcttctctc tctctctctc tctctctctg tctctcgctc |
| 14101 | tcgccctctc tctctctctt ctctctgtct ctctctctct ctctctctct ctctctctct |
| 14161 | gtcgctctcg ccctctcgct ctctctctgt ctctgtctgt gtctctctct ctccctccct |
| 14221 | ccctccctcc ctccctccct ccctcccctt ccttggcgcc ttctcggctc ttgagactta |
| 14281 | gccgctgtct cgccgtaccc cgggtcgacc ggcgggcctt ctccaccgag cggcgtgcca |
| 14341 | cagtgcccgt cgggacgagc cggacccgcc gcgtccccgt ctcggtcggc acctccgggg |
| 14401 | tcgaccagct gccgcccgcg agctccggac ttagccggcg tctgcacgtg tcccgggtcg |
| 14461 | accagcaggc ggccgccgga cgcagcggcg caccgacgga gggcgctgat tcccgttcac |
| 14521 | gcgcccgcgc ctccaccggc ctcggcccgc cgtggagctg ggaccacgcg gaactccctc |
| 14581 | tcctacattt ttttcagccc caccgcgagt ttgcgtccgc gggaccttta agagggagtc |
| 14641 | actgctgccg tcagccagta ctgcctcctc ctttttcgct tttaggtttt gcttgccttt |
| 14701 | tttttttttt tttttttttt ttttttcttt ctttctttct ttctttcttt ctttctttct |
| 14761 | ttctttcttt ctttcgctct cgctctctcg ctctctccct cgctcgtttc tttctttctc |
| 14821 | tttctctctc tctctctctc tctctctctc tctgtctctc gctctcgccc tctctctctc |
| 14881 | tttctctctc tctctgtctc tctctctctc tctctctctc tctctctctc cctccctccc |
| 14941 | tccccctccc tccctctctc cccttccttg gcgccttctc ggctcttgag acttagccgc |
| 15001 | tgtctcgccg tgtcccgggt cgaccggcgg gccttctcca ccgagcggcg tgccacagtg |
| 15061 | cccgtcggga cgagccggac ccgccgcgtc cccgtctcgg tcggcacctc cggggtcgac |
| 15121 | cagctgccgc ccgcgagctc cggacttagc cggcgtctgc acgtgtcccg ggtcgaccag |
| 15181 | caggcggccg ccggacgctg cggcgcaccg acgcgagggc gtcgattccg gttcacgcgc |
| 15241 | cggcgacctc caccggcctc ggcccgcggt ggagctggga ccacgcggaa ctccctctcc |
| 15301 | cacatttttt tcagccccac cgcgagtttg cgtccgcggg acttttaaga gggagtcact |
| 15361 | gctgccgtca gccagtaatg cttcctcctt ttttgctttt tggttttgcc ttgcgttttc |
| 15421 | tttctttctt tctttctttc tttctttctt tctttctttc tctctctctc tctctctctc |
| 15481 | tctctgtctc tctctctctg tctctctccc ctccctccct ccttggtgcc ttctcggctc |
| 15541 | gctgctgctg ctgcctctgc ctccacggtt caagcaaaca gcaagttttc tatttcgagt |
| 15601 | aaagacgtaa tttcaccatt ttggccgggc tggtctcgaa ctcccgacct agtgatccgc |
| 15661 | ccgcctcggc ctcccaaaga ctgctgggag tacagatgtg agccaccatg cccggccgat |
| 15721 | tccttccttt tttcaatctt attttctgaa cgctgccgtg tatgaacata catctacaca |
| 15781 | cacacacaca cacacacaca cacacacaca cacacacaca cacacacccc gtagtgataa |
| 15841 | aactatgtaa atgatatttc cataattaat acgtttatat tatgttactt ttaatggatg |
| 15901 | aatatgtatc gaagccccat ttcatttaca tacacgtgta tgtatatcct tcctcccttc |
| 15961 | cttcattcat tatttattaa taattttcgt ttatttattt tcttttcttt tggggccggc |
| 16021 | ccgcctggtc ttctgtctct gcgctctggt gacctcagcc tcccaaatag ctgggactac |
| 16081 | agggatctct taagcccggg aggagaggtt aacgtgggct gtgatcgcac acttccactc |
| 16141 | cagcttacgt gggctgcggt gcggtggggt ggggtggggt ggggtggggt gcagagaaaa |
| 16201 | cgattgattg cgatctcaat tgccttttag cttcattcat accctgttat ttgctcgttt |
| 16261 | attctcatgg gttcttctgt gtcattgtca cgttcatcgt ttgcttgcct gcttgcctgt |
| 16321 | ttatttcctt ccttccttcc ttccttcctt ccttccttcc ttccttcctt ccctccctta |
| 16381 | ctggcagggt cttcctctgt ctctgccgcc caggatcacc ccaacctcaa cgctttggac |
| 16441 | cgaccaaacg gtcgttctgc ctctgatccc tcccatcccc attacctgag actacaggcg |
| 16501 | cgcaccacca caccggctga cttttatgtt gtttctcatg ttttccgtag gtaggtatgt |
| 16561 | gtgtgtgtgt gtgtgtgtgt gtgtgtgtgt gtgtgtgtgt gtgtgtgtgt gtgtgtatct |
| 16621 | atgtatgtac gtatgtatgt atgtatgtga gtgagatggg tttcggggtt ctatcatgtt |
| 16681 | gcccacgctg gtctcgaact cctgtcctca agcaatccgc ctgcctgcct cggccgccca |
| 16741 | cactgctgct attacaggcg tgagacgctg cgcctggctc cttctacatt tgcctgcctg |
| 16801 | cctgcctgcc tgcctgccta tcaatcgtct tctttttagt acggatgtcg tctcgcttta |
| 16861 | ttgtccatgc tctgggcaca cgtggtctct tttcaaactt ctatgattat tattattgta |
| 16921 | ggcgtcatct cacgtgtcga ggtgatctcg aacttttagg ctccagagat cctcccgcat |
| 16981 | cggcctcccg gagtgctgtg atgacacgcg tgggcacggt acgctctggt cgtgtttgtc |
| 17041 | gtgggtcggt tctttccgtt tttaatacgg ggactgcgaa cgaagaaaat tttcagacgc |
| 17101 | atctcaccga tccgcctttt cgttctttct ttttattctc tttagacgga gtttcactct |
| 17161 | tgtcgcccag ggtggagtac gatggcggct ctcggctcac cgcaccctcc gcctcccagg |
| 17221 | ttcaagtgat tctcctgcct cagccttccc gagtagctgg aatgacagag atgagccatc |
| 17281 | gtgcccggct aatttttcta tttttagtac agatggggtt tctccatctt ggtcaggctg |
| 17341 | gtcttcaact tccgaccgtt ggagaatctt aactttcttg gtggtggttg ttttcctttt |
| 17401 | tctttttttt tcttttcttt tctttccttc tcctcccccc cccacccccc ttgtcgtcgt |
| 17461 | cctcctcctc ctcctcctcc tcctcctcct cctcctcctc ctcctcctcc tctttcattt |
| 17521 | ctttcagctg ggctctccta cttgtgttgc tctgttgctc acgctggtct caaactcctg |
| 17581 | gccttgactc ttctcccgtc acatccgccg tctggttgtt gaaatgagca tctctcgtaa |
| 17641 | aatggaaaag atgaaagaaa taaacacgaa gacggaaagc acggtgtgaa cgtttctctt |
| 17701 | gccgtctccc ggggtgtacc ttggacccgg aaacacggag ggagcttggc tgagtgggtt |
| 17761 | ttcggtgccg aaacctcccg agggcctcct tccctctccc ccttgtcccc gcttctccgc |
| 17821 | cagccgaggc tcccaccgcc gcccctggca ttttccatag gagaggtatg ggagaggact |
| 17881 | gacacgcctt ccagatctat atcctgccgg acgtctctgg ctcggcgtgc cccaccggct |
| 17941 | acctgccacc ttccagggag ctctgaggcg gatgcgaccc ccaccccccc gtcacgtccc |
| 18001 | gctaccctcc cccggctggc ctttgccggg cgaccccagg ggaaccgcgt tgatgctgct |
| 18061 | tcggatcctc cggcgaagac ttccaccgga tgccccgggt gggccggttg ggatcagact |
| 18121 | ggaccacccc ggaccgtgct gttcttgggg gtgggttgac gtacagggtg gactggcagc |
| 18181 | cccagcattg taaagggtgc gtgggtatgg aaatgtcacc taggatgccc tccttccctt |
| 18241 | cggtctgcct tcagctgcct caggcgtgaa gacaacttcc catcggaacc tcttctcttc |
| 18301 | cctttctcca gcacacagat gagacgcacg agagggagaa acagctcaat agataccgct |
| 18361 | gaccttcatt tgtggaatcc tcagtcatcg acacacaaga caggtgacta ggcagggaca |
| 18421 | cagatcaaac actatttccg ggtcctcgtg gtgggattgg tctctctctc tctctctctc |
| 18481 | tctctctctc tctctctctc tctcgcacgc gcacgcgcgc acacacacac acaatttcca |
| 18541 | tatctagttc acagagcaca ctcacttccc cttttcacag tacgcaggct gagtaaaacg |
| 18601 | cgccccaccc tccacccgtt ggctgacgaa accccttctc tacaattgat gaaaaagatg |
| 18661 | atctgggccg ggcacgctag ctcacgcctg tcactccggc actttgggag gccgaggcgg |
| 18721 | gtggatcgct tggggccggg agttcgagac caggctggcc gacgtggcga aaccccgtct |
| 18781 | ctctgaaaaa tagaacgatt agccgggcct ggtggcgtgg gcttggaatc acgaccgctc |
| 18841 | gggagactgg ggcgggcgac ttgttccaac cggggaggcc gaggccgcga tgagctgaga |
| 18901 | tcgtgccgtg gcgatgcggc ctggatgacg gagcgagacc ccgtctcgag agaatcatga |
| 18961 | tgttattata agatgagttg tgcgcggtga tggccgcctg tagtcgcggc tactcgggag |
| 19021 | gctgagacga ggagaagatc acttgaggcc ccacaggtcg aggcttcggt cggccgtgac |
| 19081 | ccactgtatc ctgggcagtc accggtcaag gagatatgcc ccttccccgt ttgcttttct |
| 19141 | tttcttccct tctcttttct tctttttgct tctcttttct ttctttcttt ctttctttct |
| 19201 | ttctttcttt ctttctttct ttttcttttt ctctcttccc ctctttcttt cctgccttcc |
| 19261 | tgcctttctt cttttcttct ttcctccctt cctcccttcc ttctttcctc ccgcctcagc |
| 19321 | ctcccaaagt gctgggatga ctggcgggag gcaccatgcc tgcttggccc aaagagaccc |
| 19381 | tcttggaaag tgagacgcag agagcgcctt ccagtgatct cattgactga tttagagacg |
| 19441 | gcatctcgct ccgtcacccc ggcagtggtg ccgtcgtaac tcactccctg cagcgtggac |
| 19501 | gctcctggac tcgagcgatc cttccacctc agcctccaga gtacagagcc tgggaccgcg |
| 19561 | ggcacgcgcc actgtgccca caccgttttt aattgttttt ttttcccccg agacagagtt |
| 19621 | tcactctcgt ggcctagact gcagtgcggt ggcgcgatct tggctcaccg caacctctgc |
| 19681 | ctcccggttt caagcgattc tcctgcatcg gcctcctgag tagccgggat tgcgggcatg |
| 19741 | cgctgccacg tctggctgat ttcgtatttt tagtggagac ggggcttctc catgtcgatc |
| 19801 | gggctggttt cgaactcccg acctcaggtg atccgccctc cccggcctcc ggaagtgctg |
| 19861 | ggatgacagg cgtgagccac cgcgcccggc cttcattttt aaatgttttc ccacagacgg |
| 19921 | ggtctcatca tttctttgca accctcctgc ccggcgtctc aaagtgctgg cgtgacgggc |
| 19981 | gtgagccact gcgcctggac tccggggaat gactcacgac caccatcgct ctactgatcc |
| 20041 | tttctttctt tctttctttc tttctttctt tctttctttc tttctttctt tctttcttga |
| 20101 | tgaattatct tatgatttat ttgtgtactt attttcagac ggagtctcgc tctgggcggg |
| 20161 | gcgaggcgag gcgaggcaca gcgcatcgct ttggaagccg cggcaacgcc tttcaaagcc |
| 20221 | ccattcgtat gcacagagcc ttattccctt cctggagttg gagctgatgc cttccgtagc |
| 20281 | cttgggcttc tctccattcg gaagcttgac aggcgcaggg ccacccagag gctggctgcg |
| 20341 | gctgaggatt agggggtgtg ttggggctga aaactgggtc ccctattttt gatacctcag |
| 20401 | ccgacacatc ccccgaccgc catcgcttgc tcgccctctg agatcccccg cctccaccgc |
| 20461 | cttgcaggct cacctcttac tttcatttct tcctttcttg cgtttgagga gggggtgcgg |
| 20521 | gaatgagggt gtgtgtgggg agggggtgcg gggtggggac ggaggggagc gtcctaaggg |
| 20581 | tcgatttagt gtcatgcctc tttcaccacc accaccacca ccgaagatga cagcaaggat |
| 20641 | cggctaaata ccgcgtgttc tcatctagaa gtgggaactt acagatgaca gttcttgcat |
| 20701 | gggcagaacg agggggaccg gggacgcgga agtctgcttg agggaggagg ggtggaagga |
| 20761 | gagacagctt caggaagaaa acaaaacacg aatactgtcg gacacagcac tgactacccg |
| 20821 | ggtgatgaaa tcatctgcac actgaacacc cccgtcacaa gtttacctat gtcacaatct |
| 20881 | tgcacatgta tcgcttgaac gacaaataaa agttaggggg gagaagagag gagagagaga |
| 20941 | gagagagaga gacagagaga gacagagaga gagagagagg agggagagag gaaaacgaaa |
| 21001 | caccacctcc ttgacctgag tcagggggtt tctggccttt tgggagaacg ttcagcgaca |
| 21061 | atgcagtatt tgggcccgtt cttttttttt cttcttcttt tctttctttt tttttggact |
| 21121 | gagtctctct cgctctgtca cccaggctgc ggtcgcggtg gcgctctctc ggctcactga |
| 21181 | aacctctgct tcccgggttc cagtgattct tcttcggtag ctgggattac aggcgcacac |
| 21241 | catgacggcg ggctcatatt cctattttca gtagagacgg ggtttctcca cgttggccac |
| 21301 | gctggtctcg aactcctgac ctcaaatgat ccgccttcct gggcctccca aagtgctgga |
| 21361 | aacgacaggc ctgagccgcc gggatttcag cctttaaaag cgcggccctg ccacctttcg |
| 21421 | ctgtggccct tacgctcaga atgacgtgtc ctctctgccg taggttgact ccttgagtcc |
| 21481 | cctaggccat tgcactgtag cctgggcagc aagagccaaa ctccgnnccc ccacctcctc |
| 21541 | gcgcacataa taactaacta acaaactaac taactaacta aactaactaa ctaactaaaa |
| 21601 | tctctacacg tcacccataa gtgtgtgttc ccgtgagagt gatttctaag aaatggtact |
| 21661 | gtacactgaa cgcagtggct cacgtctgtc atcccgaggt caggagttcg agaccagccc |
| 21721 | ggccaacgtg gtgaaacccc gtctctactg aaaatacgaa atggagtcag gcgccgtggg |
| 21781 | gcaggcacct gtaaccccag ctactcggga ggctggggtg gaagaattgc ttgaacctgg |
| 21841 | caggcggagg ctgcagtgac ccaagatcgc accactgcac tacagcctgg gcgacagagt |
| 21901 | gagacccggt ctccagataa atacgtacat aaataaatac acacatacat acatacatac |
| 21961 | atacatacat acatacatac atccatgcat acagatatac aagaaagaaa aaaagaaaag |
| 22021 | aaaagaaaga gaaaatgaaa gaaaaggcac tgtattgcta ctgggctagg gccttctctc |
| 22081 | tgtctgtttc tctctgttcg tctctgtctt tctctctgtg tctctttctc tgtctgtctg |
| 22141 | tctctttctt tctctctgtc tctgtctctg tctttgtctc tctctctccc tctctgcctg |
| 22201 | tctcactgtg tctgtcttct gtcttactct ctttctctcc ccgtctgtct ctctctctct |
| 22261 | ctctccctcc ctgtttgttt ctctctctcc ctccctgtct gtttctctct ctctctttct |
| 22321 | gtctgtttct gtctctctct gtctgtctat gtctttctct gtctgtctct ttctctgtct |
| 22381 | gtctgcctct ctctttcttt ttctgtgtct ctctgtcggt ctctctctct ctgtctgtct |
| 22441 | gtctgtctct ctctctctct ctctgtgcct atcttctgtc ttactctctt tctctgcctg |
| 22501 | tctgtctgtc tctccctccc tttctgtttc tctctctctc tctctctctc tccccctctc |
| 22561 | cctgtctgtt tctctccgtc tctctctctt tctgtctgtt tctcactgtc tctctctgtc |
| 22621 | catctctctc tctctctgtc tgtctctttc gttctctctg tctgtctgtc tctctctctc |
| 22681 | tctctctctc tctctctctc tccctgtctg tctgtttctc tctatctctc gctgtccatc |
| 22741 | tctgtctttc tatgtctgtc tctttctctg tcagtctgtc agacaccccc gtgccgggta |
| 22801 | gggccctgcc ccttccacga aagtgagaag cgcgtgcttc ggtgcttaga gaggccgaga |
| 22861 | ggaatctaga caggcgggcc ttgctgggct tccccactcg gtgtatgatt tcgggaggtc |
| 22921 | gaggccgggt ccccgcttgg atgcgagggg cattttcaga cttttctctc ggtcacgtgt |
| 22981 | ggcgtccgta cttctcctat ttccccgata agctcctcga cttcaacata aacggcgtcc |
| 23041 | taagggtcga tttagtgtca tgcctctttc accgccacca ccgaagatga aagcaaagat |
| 23101 | cggctaaata ccgcgtgttc tcatctagaa gtgggaactt acagatgaca gttcttgcat |
| 23161 | gggcagaacg agggggaccg ggnacgcgga agcctgcttg agggrggagg ggyggaagga |
| 23221 | gagacagctt caggaagaaa acaaaacacg aatactgtcg gacacagcac tgactacccg |
| 23281 | ggtgatgaaa tcatctgcac actgaacacc cccgtcacaa gtttacctat gtcacagtct |
| 23341 | tgctcatgta tgcttgaacg acaaataaaa gttcgggggg gagaagagag gagagagaga |
| 23401 | gagagacggg gagagagggg ggagaggggg ggggagagag agagagagag agagagagag |
| 23461 | agagagagag agaaagagaa gtaaaaccaa ccaccacctc cttgacctga gtcagggggt |
| 23521 | ttctggcctt ttgggagaac gttcagcgac aatgcagtat ttgggcccgt tctttttttc |
| 23581 | ttcttcttct tttctttctt tttttttgga ctgagtctct ctcgctctgt cacccaggct |
| 23641 | gcggtgcggt ggcgctctct cggctcactg aaacctctgc ttcccgggtt ccagtgattc |
| 23701 | ttcttcggta gctgggatta caggtgcgca ccatgacggc cggctcatcg ttctattttt |
| 23761 | agtagagacg gggtttctcc acgttggcca cgctggtctc gaactcctga ccacaaatga |
| 23821 | tccaccttcc tgggcctccc aaagtgctgg aaacgacagg cctgagccgc cgggatttca |
| 23881 | gcctttaaaa gcgcgcggcc ctgccacctt tcgctgcggc ccttacgctc agaatgacgt |
| 23941 | gtcctctctg ccataggttg actccttgag tcccctaggc cattgcactg tagcctgggc |
| 24001 | agcaagagcc aaactccgtc cccccacctc cccgcgcaca taataactaa ctaactaact |
| 24061 | aactaactaa aatctctaca cgtcacccat aagtgtgtgt tcccgtgagg agtgatttct |
| 24121 | aagaaatggt actgtacact gaacgcaggc ttcacgtctg tcatcccgag gtcaggagtt |
| 24181 | cgagaccagc ccggcccacg tggtgaaacc cccgtctcta ctgaaaatac gaaatggagt |
| 24241 | caggcgccgt ggggcaggca cctgtaaccc cagctactcg ggaggctggg gtggaagaat |
| 24301 | tgcttgaacc tggcaggcgg aggctgcagt gacccaagat cgcaccactg cactacagcc |
| 24361 | tgggcgacag agtgagaccc ggtctccaga taaatacgta cataaataaa tacacacata |
| 24421 | catacataca tacatacaac atacatacat acagatatac aagaaagaaa aaaagaaaag |
| 24481 | aaaagaaaga gaaaatgaaa gaaaaggcac tgtattgcta ctgggctagg gccttctctc |
| 24541 | tgtctgtttc tctctgttcg tctctgtctt tctctctgtg tctctttctc tgtctgtctg |
| 24601 | tctgtctgtc tgtctgtctc tttctttctt tctgtctctg tctttgtccc tctctctccc |
| 24661 | tctctgccct gtctcactgt gtctgtcttc tatcttactc tctttctctc cccgtctgtc |
| 24721 | tctctctcac tccctccctg tctgtttctc tctctctctc tttctgtctg tttctgtctc |
| 24781 | tctctgtctg cctctctctt tctctatctg tctctttctc tgtctgtctg cccctctctt |
| 24841 | tctttttctg tgtctctctg tctgtctctc tctctctctg tgcctatctt ctgtcttact |
| 24901 | ctctttctct gcctgtctgt ctgtctctct ctgtctctcc ctccctttct gcttctctct |
| 24961 | ctctctctct ctctnnnccc tccctgtctg tttctctctg tctccctctc tttctgtctg |
| 25021 | tttctcactg tctctctctg tctgtctgtt tcattctctc tgtctctgtc tctgtctctc |
| 25081 | tctctctctg tctctccctc tctgtgtgta tcttttgtct tactctcctt ctctgcctgt |
| 25141 | ccgtctgtct gtctgtctct ctctctccct gtccctctct ctttctgtct gtttctctct |
| 25201 | ctctctctct ctctctctct ctgtctctgt ctttctctgt ctgtcccttt ctctgtctgt |
| 25261 | ctgcctctct ctttctcttt ctgtgtctct ctgtctctct ctctgtgcct atcttctgtc |
| 25321 | ttactctctt tctctgcctg tctatctgtc tgtctctctc tgtctctctc cctgcctttc |
| 25381 | tgtttctctc tctctccctc tctcgctctc tctgtctttc tctctttctc tctgtttctc |
| 25441 | tgtctctctc tgtccgtctc tgtctttttc tgtctgtctg tctctctctt tctttctgtc |
| 25501 | gtctgtctct gtctctgtct ctgtctctct ctctctctct ctccttgtct ctctcactgt |
| 25561 | gtctgtcttc tgtcttactc tccttctctg cctgtccatc tgtctgtctg tctctctctc |
| 25621 | tctctcccta cctttctgtt tctctctcgc tagctctctc tctctctgcc tgtttctctc |
| 25681 | tttctctctc tgtctttctc tgtctgtctc tttctctgtc tgtctgtctc tttctctctg |
| 25741 | tctctgtctc tgtctctctc tctctctctc tctctctctc tgcctctctc actgtgtctg |
| 25801 | tcttctgtct tattctcttt ctctctctgt ctctctctct ctctccttta ctgtctgttt |
| 25861 | ctctctctct ctctctcttt ctgcctgttt ctctctgtct gtctctgtct ttctctgtct |
| 25921 | gtctgcctct ctctttcttt ttctgcgtct ctctgtctct ctctctctct ctctgttcct |
| 25981 | atcttctgtc ttactctgtt tccttgcctg cctgcctgtc tgtgtgtctg tctctctctc |
| 26041 | tctctctctc tctctctccc tccctttctc tttctctgtc tctctctctc tttctgggtg |
| 26101 | tttctctctg tctctctgtc catctctgtc tttctatgtc tgtctctctc tttctctctg |
| 26161 | tctctgtctc tgcctctctc tctctctctc tctctctctc tctgtctgtc tctctcactg |
| 26221 | tgtgtgtctg tcttctgtct tactctcctt ctctgcctgt ccgtctgtct gtctgtctct |
| 26281 | ccctctctct ccctcccttt ctgtttctct ctctctctct ttctgtctgt ttctctcttt |
| 26341 | ctctctctgt ctgtctcttt ctctgtctgt ctgtctctct ctttcttttt ctctgtctct |
| 26401 | ctgtctctct ctgtgtctgt ctctctgtct gtgcctatct tctgtcttac tctctttctc |
| 26461 | tggctgtctg cctgtctctc tctctctctc tgtctgtctc cgtccctctc tccctgtctg |
| 26521 | tctgtttctc tctctgcctc tctctctctc tgtctgtctc tttctctgtc tgtctgtctc |
| 26581 | tctctttctt tttctctgtc tctctgtctc tctctgtgtc tgtctctctt tctgtgccta |
| 26641 | tcttctgtct tactctcttt ctctggctgt ctgcctgtct ctctctctct gcctgtctcc |
| 26701 | gtccctccct ccctgtctgt ctgtttctct ctctgtctct gtctctctgt ccatctctgt |
| 26761 | ctgtctcttt ctctttctct ctctctgtct ctgtctctct ctctctctgc ctgtctctct |
| 26821 | cactgtgtct gtcttctgtc ttactctctt tctcttgcct gcctctctgt ctgtctgtct |
| 26881 | ctctccctcc atgtctctct ctctctctca ctcactctct ctccgtctct ctctctttct |
| 26941 | gtctgtttct ctctctgtct gtctctctcc ctccatgtct ctctctctct ctctcactca |
| 27001 | ctctctctcc gtctctctct ctctttctgt ctgtttctct ctctgtctgt ctctctccct |
| 27061 | ccatgtctct ctctctccct ctcactcact ctctctccgt ctctctctct ctttctgtct |
| 27121 | gtttctttgt ctgtctgtct gtctgtctgt ctgtctctct ctctctctct ctctctctct |
| 27181 | ctctctgttt gtctttctcc ctccctgtct gtctgtctgt ctctctctct ctgtctctgt |
| 27241 | ctctgtctct ctctctttct ctttctgtct gtttctctct atctctcgct gtccatctct |
| 27301 | gtctttctat gtctgtctct ttctctgtca gtctgtcaga cacacccgtg ccggtagggc |
| 27361 | cctgcccttc cacgagagtg agaagcgcgt gcttcggtgc ttagagaggc cgagaggaat |
| 27421 | ctagacaggc gggccttgct gggcttcccc actcggtgta cgatttcggg aggtcgaggc |
| 27481 | cgggtccccg cttggatgcg aggggcattt tcagactttt ctctcggtca cgtgtggcgt |
| 27541 | ccgtacttct cctatttccc cgataagtct cctcgacttc aacataaact gttaaggccg |
| 27601 | gacgccaaca cggcgaaacc ccgtctctac taaaaataca aagctgagtc gggagcggtg |
| 27661 | gggcaggccc tgtaatgcca gctcctcggg aggctgaggc gggagaatcg cttgaaccag |
| 27721 | ggaagcggag gctgcaggga gccgagatcg cgccactgca ctacggccca ggctgtagag |
| 27781 | tgagtgagac tcggtctcta aataaatacg gaaattaatt aattcattaa ttcttttccc |
| 27841 | tgctgacgga catttgcagg caggcatcgg ttgtcttcgg gcatcaccta gcggccactg |
| 27901 | ttattgaaag tcgacgttga cacggaggga ggtctcgccg acttcaccga gcctggggca |
| 27961 | acgggtttct ctctctccct tctggaggcc cctccctctc tccctcgttg cctagggaac |
| 28021 | ctcgcctagg gaacctccgc cctgggggcc ctattgttct ttgatcggcg ctttactttt |
| 28081 | ctttgtgttt tggcgcctag actcttctac ttgggctttg ggaagggtca gtttaatttt |
| 28141 | caagttgccc cccggctccc cccactaccc acgtcccttc accttaattt agtgagncgg |
| 28201 | ttaggtgggt ttcccccaaa ccgccccccc ccccccgcct cccaacaccc tgcttggaaa |
| 28261 | ccttccagag ccaccccggt gtgcctccgt cttctctccc cttcccccac cccttgccgg |
| 28321 | cgatctcatt cttgccaggc tgacatttgc atcggtgggc gtcaggcctc actcgggggc |
| 28381 | caccgttttt gaagatgggg gcggcacggt cccacttccc cggaggcagc ttgggccgat |
| 28441 | ggcatagccc cttgacccgc gtgggcaagc gggcgggtct gcagttgtga ggcttttccc |
| 28501 | cccgctgctt cccgctcagg cctccctccc taggaaagct tcaccctggc tgggtctcgg |
| 28561 | tcacctttta tcacgatgtt ttagtttctc cgccctccgg ccagcagagt ttcacaatgc |
| 28621 | gaagggcgcc acggctctag tctgggcctt ctcagtactt gcccaaaata gaaacgcttt |
| 28681 | ctgaaaacta ataactttnc tcacttaaga tttccaggga cggcgccttg gcccgtgttt |
| 28741 | gttggcttgt tttgtttcgt tctgttttgt tttgttcgtg tttttccttt ctcgtatgtc |
| 28801 | tttcttttca ggtgaagtag aaatccccag ttttcaggaa gacgtctatt ttccccaaga |
| 28861 | cacgttagct gccgtttttt cctgttgtga actagcgctt ttgtgactct ctcaacgctg |
| 28921 | cagtgagagc cggttgatgt ttacnatcct tcatcatgac atcttatttt ctagaaatcc |
| 28981 | gtaggcgaat gctgctgctg ctcttgttgc tgttgttgtt gttgttgttg tcgtcgttgc |
| 29041 | tgttgtcgtt gtcgttgttg ttgtcgttgt cgttgttttc aaagtatacc ccggccaccg |
| 29101 | tttatgggat caaaagcatt ataaaatatg tgtgattatt tcttgagcac gcccttcctc |
| 29161 | cccctctctc tgtctctctg tctgtctctg tctctctctt tctctgtctg tcttctctct |
| 29221 | ctctctctct ctgtgtctct ctctctctgc ctgtctgttt ctctctctct gcctctctct |
| 29281 | ctctctctct ctctgcctgt ctctctcact gtgtctgtct tctgtcttac tccctttctc |
| 29341 | tgtctgtctg tcggtctctc tctctctctc tccctgtctg tatgtttctc tctgtctctg |
| 29401 | tctctctctc tctttctgtt tctctctctc cgtctctgtc tttctctgac tgtctctctc |
| 29461 | tttccttctc tctgtctctc tctgcctgtc tctctcactc tgtcttctgt cttatctctc |
| 29521 | tctctgcctg cctgtctctc tcactctctc tctctgtgtg tctctctctc tctttctgtt |
| 29581 | tctctctgtc tctctgtccg tctctgtctt tctctgtctg tctctttgtc tgtctgtctt |
| 29641 | tgtctttcct tctctctgtc tctgtctctc tcactgtgtc tgtcttctgt cttagtctct |
| 29701 | ctctctctct ctccctgtct gtctgtctct ctctctctct ccccctgtct gtttctctct |
| 29761 | ctctctctct ctctctctct ctctgtcttt gtctttcttt ctgtctctgt ctctctctct |
| 29821 | ctctctgtgt gtctgtcttc tgtcttactg tctttctctg cctgtctgtc tgtctgtctc |
| 29881 | tctctgtctg tctctctctc tctctccccc tgtcggctgt ttctctgtct ctgtctgtgt |
| 29941 | ctctctttct gtctgtttct ctctgtctgt ctttctctct ctgtctcttt ctctctgtct |
| 30001 | ctctgtctgt ctctgtctct ctctctgtct ctctctctct gtgggggtgt gtgtgtgtgt |
| 30061 | gtgtatgtgt gtgtgtgtgt gtgtgtgtgt ctgccttctg tcttactctc tttctctgcc |
| 30121 | tgtctgtctg cctgtctgtt tgtctctctc tctctgcctg tctctctccc ttcctgtctg |
| 30181 | tttctctctc tttctgtttc tctctgtctc tgtccatctc tgtctttctc cgtctgtctc |
| 30241 | tttatctgtc tctctccgtc tgtctcttta tctgtctctc tctctctttc tgtctttctc |
| 30301 | tctctgtgta tcgttgtctc tctctgtctg tctctgtctc tgtctctctg tctctctctc |
| 30361 | tctctctctc tctctgtctg tctgtccgtc tgtctgtctc ggtctctgcg tctcgctatc |
| 30421 | tcccgccctc tctttttttg caaaagaagc tcaagtacat ctaatctaat cccttaccaa |
| 30481 | ggcctgaatt cttcacttct gacatcccag atttgatctc cctacagaat gctgtacaga |
| 30541 | actggcgagt tgatttctgg acttggatac ctcatagaaa ctacatatga ataaagatcc |
| 30601 | aatcctaaaa tctggggtgg cttctccctc gactgtctcg aaaaatcgta cctctgttcc |
| 30661 | cctaggatgc cggaagagtt ttctcaatgt gcatctgccc gtgtcctaag tgatctgtga |
| 30721 | ccgagccctg tccgtcctgt ctcaaatatg tacgtgcaaa cacttctctc catttccaca |
| 30781 | actacccacg gccccttgtg gaaccactgg ctctttgaaa aaaatcccag aagtggtttt |
| 30841 | ggctttttgg ctaggaggcc taagcctgct gagaactttc ctgcccagga tcctcgggac |
| 30901 | catgcttgct agcgctggat gagtctctgg aaggacgcac gggactccgc aaagctgacc |
| 30961 | tgtcccaccg aggtcaaatg gatacctctg cattggcccg aggcctccga agtacatcac |
| 31021 | cgtcaccaac cgtcaccgtc agcatccttg tgagcctgcc caaggccccg cctccgggga |
| 31081 | gactcttggg agcccggcct tcgtcggcta aagtccaaag ggatggtgac ttccacccac |
| 31141 | aaggtcccac tgaacggcga agatgtggag cgtaggtcag agaggggacc aggaggggag |
| 31201 | acgtcccgac aggcgacgag ttcccaaggc tctggccacc ccacccacgc cccacgcccc |
| 31261 | acgtcccggg cacccgcggg acaccgccgc tttatcccct cctctgtcca cagccggccc |
| 31321 | caccccacca cgcaacccac gcacacacgc tggaggttcc aaaaccacac ggtgtgacta |
| 31381 | gagcctgacg gagcgagagc ccatttcacg aggtgggagg ggtgggggtg gggtgggttg |
| 31441 | ggggttgtgg ggtctgtggc gagcccgatt ctccctcttg ggtggctaca ggctagaaat |
| 31501 | gaatatcgct tcttgggggg aggggcttcc ttaggccatc accgcttgcg ggactacctc |
| 31561 | tcaaaccctc ccttgaggcc acaaaataga ttccacccca cccatcgacg tttcccccgg |
| 31621 | gtgctggatg tatcctgtca agagacctga gcctgacacc gtcgaattaa acaccttgac |
| 31681 | tggctttgtg tgtttgtttg tttctgagat ggagtcttgc tctgtccccc aggctggagt |
| 31741 | gcagtggcgt gatctcagct cactggaacc tctgcctcct gggttcaagt gattctcctg |
| 31801 | tctcagcgcc accatggccg gctcattttt tttttttttt tttttggtag acacggggtt |
| 31861 | tcaccctctt tcattggttt tcactggaga ttctagattc gagccacacc tcattccgtg |
| 31921 | ccacagagag acttcttttt tttttttttt tttttaagcg caacgcaaca tgtctgcctt |
| 31981 | atttgagtgg cttcctatat cattataatt gtgttataga tgaagaaacg gtattaaaca |
| 32041 | ctgtgctaat gatagtgaaa gtgaagacaa aagaaaggct atctattttg tggttagaat |
| 32101 | aaagttgctc agtatttaga agctacctaa atacgtcagc atttacactc ttcctagtaa |
| 32161 | aagctggccg atctgaataa tcctccttta aacaaacaca atttttgata gggttaagat |
| 32221 | ttttttaaga atgcgactcc tgcaaaatag ctgaacagac gatacacatt taaaaaaata |
| 32281 | acaacacaag gatcaaccag acttgggaaa aaatcgaaaa ccacacaagt cttatgaaga |
| 32341 | actgagttct taaaatagga cggagaacgt agctatcgga agagaaggca gtattggcaa |
| 32401 | gttgattgtt acgttggtca gcagtagctg gcactatctt tttggccatc tttcgggcaa |
| 32461 | tgtaactact acagcaaaat gagatatgat ccattaaaca acatattcgc aaatcaaaaa |
| 32521 | gtgtttcagt aatataatgc ttcagattta gaagcaaatc aaatgataga actccactgc |
| 32581 | tgtaataagt caccccaaag atcaccgtat ctgacaaaat aactaccaca gggttatgac |
| 32641 | ttcagaatca tactttcttc ttgatattta cttatgtatt tatttttttt aatttatttc |
| 32701 | tcttgagacg cgtctcgctc tgtcgcccag gctggagtgc gatggtgtga tctcggctca |
| 32761 | ctgcaaccgc cacctccctg ggttcaagcg attctcctgc ctcagcctcc cgagtagctg |
| 32821 | ggactacagg tgcccgccac cacgcccagc taatctttat acttttaata gagacggggt |
| 32881 | ttcaccgtgt cggcccggat ggtctcgatc tcttgacctc gtgacccgcc cgcctcggcc |
| 32941 | tcccaaagtg ctgggatgac aggcgtgagc cactgagccc ggccttctct tgacgtttaa |
| 33001 | actatgaagt cagtccagag aaacgcaata aatgtcaacg gtgaggatgg tgttgaggca |
| 33061 | gaagtaggac cacacttttt cctatcttat tcagttgata acaatatgac ctaggtagta |
| 33121 | atttcctatg tgcctactta tacacgagta caaaagagta aaacagagag actgctaaat |
| 33181 | taaagggtac gtgaagttct tcatagtaac tccgtaaact ggaacactgt caaaaagcag |
| 33241 | cagctagtga attgtttcca tgtatttttc tattatccaa taagtgaact atgctattcc |
| 33301 | tttccagtct cccaagcact tcttgtcccc atcaccactt cggtgctcga agaaaaagta |
| 33361 | agcaaatcaa ggaacacaag ctaaagaaac acacacacaa accaaagaca actacagcgt |
| 33421 | ctgcaaaagt ttgctagaag actgaaactg ttgagtataa ggatctggta ttctacgatc |
| 33481 | atgagttcac ttcagagttt gttcaagaca tacgtttcgt aaggaaacat cttagttaga |
| 33541 | agttattcag cagtaggtac catccctaag tatttttcac caaatccgtg acaataaaga |
| 33601 | gctatctaac cagaaaaatt agcgagtacg ggcaccatcc atagggcttt gtctttacgc |
| 33661 | ttcattagca cttaccatgc cttacaatgt ctaggattga ccctgatagc atttcgaaaa |
| 33721 | caagctaatg ctttgtccag ttcttcagtg aagacaactc acgccctaat gcgctatagg |
| 33781 | cataagcatc atttggatcc acttcgagag ttctctggaa gaattgaatc gcaatatcgt |
| 33841 | gttcccgttt gcagaccgaa acagtttccc tgcagcacac caggcctctg gctggcgaat |
| 33901 | ttttatccat gtctgtgaag tctttggaca gaactgaaag agcaacctct ttcggaggat |
| 33961 | gccaaagtgt tgtagagtag atctccatgc cttcgactct gtaattctca atcctcctaa |
| 34021 | cctctgagaa ttgtctttca gcttgcgtgg actctgaaag tttacaatag gccntttccg |
| 34081 | atttggcaca gtacccaacc ggtattgcag tggtgagaag ctagatggct caagatgctg |
| 34141 | atagcttctt tgccgtggta agaacacaaa gctaaataac ctttccccct ttcacgaaga |
| 34201 | aggctcatca agccttccgc tgctgctttt tgtagattaa aagcctgaat ctgaggcgcg |
| 34261 | attgcggcta ttttcccttc tgaaatgacg gaagagtcca attttgtcac ttccaggcta |
| 34321 | tcacttatgt tcggtggagt tattgctcct ttattagttt tacttttggt tcttctgttt |
| 34381 | gggattttag gtggaaactt catttttaat tttctcctaa ttctcctcgg ttgtggagct |
| 34441 | gtcactagtc aagagtcgtg aatttcttcg aggncggtgc atttggggga gatgccatag |
| 34501 | tggggctcaa tacctgaggt gttgcccttg tcggcggacc agaactttgt gtttttgcaa |
| 34561 | ggactggagt tacctttcgg ctctttcccc tctgcgagaa gacagacggt gttccggttt |
| 34621 | ggccgattct ggcaacaggc ttttctgaag gggctccggt ggatggcacg tcagtgacag |
| 34681 | acggtgtctc ataccagtgc agttttgtca atagggtccg tctccgggac ttggggtttc |
| 34741 | taatggcaaa atgccaacac ttggggttaa tggactaaca gctgctggtc ctcctaataa |
| 34801 | acttcgacca gtttttggtt tatgttgaac ctgtttagat catatggaag ttcctgttcc |
| 34861 | cagtgggaca gtatcaggtg aaaggacagc tgaatcgata gaagacactg gggagtctgt |
| 34921 | attcaaggag tactttgaat tggaagattc taaattccat ccgtttcatt cgacggtgtc |
| 34981 | ctggggtgtt tccgtaagaa cggtctcggg ctgtctgtga cataaactag gacgaggtcc |
| 35041 | aagtgttgtg gcgcaacact tggacaggca gttgctaaag ctctctagag aggtgaatca |
| 35101 | aaatgtttgg tcaggatctg gcttttcccc cctatttcac atcatgattc aaagggacac |
| 35161 | cagaggaaag gatttcaacg aaggctcttt tggtcacatt ctgatccttt ggtaagccga |
| 35221 | tctgtcttgc aatatacatg tcccgacgat ggaaggggaa agcgagctga atcaccaaac |
| 35281 | tcaggaacga taatatcatc gtggcttttc tgcttatgaa acactccacc cgataagatt |
| 35341 | tgatcccctt ctgcaagctt gctgagatca acacaacatt tcgcaagcag gcatttgcat |
| 35401 | tgcggggtag tacaactgtg tcctttcaag agtctatatg ttttataggc ctttcctgag |
| 35461 | cggtaagaac aggtcgccag taagaacaag gcttcttctg agtgtacttc tgcataaagg |
| 35521 | cgttctgcgg gggaaaccgc atctcggtag gcatagtggt ttagtgcttg ccatatagca |
| 35581 | gcctggacgg gtccctgcag caccgccatc ctcgaggctc aggcccactt tctgcagtgc |
| 35641 | cacaggcacc cccccccccc catagcggct ccggcccggc cagccccggc tcatttaaag |
| 35701 | gcaccagccg ccgttaccgg gggatggggg agtccgagac agaatgactt ctttatcctg |
| 35761 | ctgactctgg aaagcccggc gccttgtgat ccattgcaaa ccgagagtca cctcgtgttt |
| 35821 | agaacacgga tccactccca agttcagtgg ggggatgtga ggggtgtggc aggtaggacg |
| 35881 | aaggactctc ttccttctga ttcggtctgc acagtggggc ctagggctgg agctctctcc |
| 35941 | gtgcggaccg ctgactccct ctaccttggg ttccctcggc cccaccctgg aacgccgggc |
| 36001 | cttggcagat tctggccctt tctggccctt cagtcgctgt cagaaacccc atctcatgct |
| 36061 | cggatgcccc gagtgactgt ggctcgcacc tctccggaaa cattggaaat ctctcctcta |
| 36121 | cgcgcggcca cctgaaacca caggagctcg ggacacacgt gctttcggga gagaatgctg |
| 36181 | agagtctctc gccgactctc tcttgacttg agttcttcgt gggtgcgtgg ttaagacgta |
| 36241 | gtgagaccag atgtattaac tcaggccggg tgctggtggc tcacgcctgt aaccccaaca |
| 36301 | ctttgggagg ccgaggccgt aggatccctc gaggaatcgc ctaaccctgg ggaggttgag |
| 36361 | gttgcagtga gtgagccata gttgtgtcac tgtgctccag tctgggcgaa agacagaatg |
| 36421 | aggccctgcc acaggcaggc aggcaggcag gcaggcagaa agacaacagc tgtattatgt |
| 36481 | tcttctcagg gtaggaagca aaaataacag aatacagcac ttaattaatt tttttttttt |
| 36541 | ccttcggacg gagtttcact cttggtgccc acgctggagt gcagtggcac catctcggct |
| 36601 | caccgcaacc tccacctccc gcgttcaagc gattctcctg cctcagcctc ctgagtagct |
| 36661 | gggattacag ggaggagcca ccacacccag ctgattttgt attgttagta gagacggcat |
| 36721 | ttctccatgt gggtcaggct ggtctcgaac tggcgacccc agtggatctg cccgccccgg |
| 36781 | cctcccaaag tgctggggtg acaggcgtga gccatcgtga ctggccggct acgtttattt |
| 36841 | atttattttt ttaattattt tacttttttt tagttttcca ttttaatcta tttatttatt |
| 36901 | tacatttatt tatttattta tttatttact tatttattta ttttcgagac agactctcgc |
| 36961 | tctgctgccc aggctggagt gcagcggcgt gatctcggct cactgcaacg tccgcctccc |
| 37021 | gggttcacgc cattctcctg cctcagcctc ccaagtagct gggactacag gcgcccgcca |
| 37081 | ccgtgcccgg ctaacttttt gtattttgag tagagatggg gtttcactgt ggtagccagg |
| 37141 | atggtctcga tctcctgacc ccgtgatccg tccacctcgg cctcccaaag tgctgggatg |
| 37201 | acaggcgtga gccaccggcc ccggcctatt tatctattta ttaactttga gtccaggtta |
| 37261 | tgaaaccagt tagtttttgt aatttttttt tttttttttt ttttttgaga cgaggtttca |
| 37321 | ccgtgttgcc aaggcttgga ccgagggatc caccggccct cggcctccca aaagtgcggg |
| 37381 | gatgacaggc gcgagcctac cgcgcccgga cccccccttt ccccttcccc cgcttgtctt |
| 37441 | cccgacagac agtttcacgg cagagcgttt ggctggcgtg cttaaactca ttctaaatag |
| 37501 | aaatttggga cgtcagcttc tggcctcacg gactctgagc cgaggagtcc cctggtctgt |
| 37561 | ctatcacagg accgtacacg taaggaggag aaaaatcgta acgttcaaag tcagtcattt |
| 37621 | tgtgatacag aaatacacgg attcacccaa aacacagaaa ccagtctttt agaaatggcc |
| 37681 | ttagccctgg tgtccgtgcc agtgattctt ttcggtttgg accttgactg agaggattcc |
| 37741 | cagtcggtct ctcgtctctg gacggaagtt ccagatgatc cgatgggtgg gggacttagg |
| 37801 | ctgcgtcccc ccaggagccc tggtcgatta gttgtgggga tcgccttgga gggcgcggtg |
| 37861 | acccactgtg ctgtgggagc ctccatcctt ccccccaccc cctccccagg gggatcccaa |
| 37921 | ttcattccgg gctgacacgc tcactggcag gcgtcgggca tcacctagcg gtcactgtta |
| 37981 | ctctgaaaac ggaggcctca cagaggaagg gagcaccagg ccgcctgcgc acagcctggg |
| 38041 | gcaactgtgt cttctccacc gcccccgccc ccacctccaa gttcctccct cccttgttgc |
| 38101 | ctaggaaatc gccactttga cgaccgggtc tgattgacct ttgatcaggc aaaaacgaac |
| 38161 | aaacagataa ataaataaaa taacacaaaa gtaactaact aaataaaata agtcaataca |
| 38221 | acccattaca atacaataag atacgatacg ataggatgcg ataggatacg ataggataca |
| 38281 | atacaatagg atacgataca atacaataca atacaataca atacaataca atacaataca |
| 38341 | atacaataca atacaatacg ccgggcgcgg tggctcatgc ctgtcatccc gtcactttgg |
| 38401 | gatgccgagg tggacgcatc acctgaagtc gggagttgga gacaagcccg accaacatgg |
| 38461 | agaaatcccg tctcaattga aaatacaaaa ctagccgggc gcggtggcac atgcctataa |
| 38521 | tcccagctgc taggaaggct gaggcaggag aatcgcttga acctgggaag cggaggttgc |
| 38581 | agtgagccga gattgcgcca tcgcactcca gtctgagcaa caagagcgaa actccgtctc |
| 38641 | aaaaataaat acataaataa atacatacat acatacatac atacatacat acatacatac |
| 38701 | ataaattaaa ataaataaat aaaataaaat aaataaatgg gccctgcgcg gtggctcaag |
| 38761 | cctgtcatcc cctcactttg ggaggccaag gccggtggat caagaggcgg tcagaccaac |
| 38821 | agggccagta tggtgaaacc ccgtctctac tcacaataca caacattagc cgggcgctgt |
| 38881 | gctgtgctgt actgtctgta atcccagcta ctcgggaggc cgagctgagg caggagaatc |
| 38941 | gcttgaacct gggaggcgga ggttgcagtg agccgagatc gcgccactgc aacccagcct |
| 39001 | gggcgacaga gcgagactcc gtctccaaaa aatgaaaatg aaaatgaaac gcaacaaaat |
| 39061 | aattaaaaag tgagtttctg gggaaaaaga agaaaagaaa aaagaaaaaa acaacaaaac |
| 39121 | agaacaaccc caccgtgaca tacacgtacg cttctcgcct ttcgaggcct caaacacgtt |
| 39181 | aggaattatg cgtgatttct ttttttaact tcattttatg ttattatcat gattgatgtt |
| 39241 | tcgagacgga gtctcggagg cccgccctcc ctggttgccc agacaacccc gggagacaga |
| 39301 | ccctggctgg gcccgattgt tcttctcctt ggtcaggggt ttccttgtct ttcttcgtgt |
| 39361 | ctttaacccg cgtggactct tccgcctcgg gtttgacaga tggcagctcc actttaggcc |
| 39421 | ttgttgttgt tggggacttt cctgattctc cccagatgta gtgaaagcag gtagattgcc |
| 39481 | ttgcctggcc ttgcctggcc ttgccttttc tttctttctt tctttcttta ttactttctc |
| 39541 | tttttcttct tcttcttctt cttttttttg agacagagtt tcactcttgt tgcccaggct |
| 39601 | agagggcaat ggcgcgatct cggctcaccg caccctccgc ctcccaggtt caagcgattc |
| 39661 | tcctgcctca gcctcctgat tagctgggat tacaggcatg ggccaccgtg ctggctgatg |
| 39721 | tttgtacttt tagtagagac ggtgtttttc catgttggtc aggctggtct cccactccca |
| 39781 | acctcaggtg gtccgcctgc cttagcctcc caaagtgctg ggatgacagg cgtgcaaccg |
| 39841 | cgcccagcct ctctctctct ctctctctct ctcgctcgct tgcttgcttg ctttcgtgct |
| 39901 | ttcttgcttt cccgttttct tgctttcttt ctttctttcg tttctttcat gcttgctttc |
| 39961 | ttgcttgctt gcttgctttc gtgctttctt gctttcctgt tttctttctt tctttctttc |
| 40021 | tttctttctt ttgtttcttt cttgcttgct ttcttgcttg cttgcttgct ttcgtgcttt |
| 40081 | cttgctttcc tgttttcttt ctttctttct ttcttttctt tctttcttgc ttgctttcct |
| 40141 | gcttgcttgc tttcgtgctt tcttgttttc tcgatttctt tctttctttt gtttctttcc |
| 40201 | tgcttgcttt cttgcttgct tgctttcgtg cttcttgctt tcctgttttc tttctttctt |
| 40261 | tctttctttt gtttctttct tgcttgcttt cttgcttgct tgctttcgtg ctgtcttgtt |
| 40321 | tctcgatttc tttctttctt ttgtttcttt cctgcttgct ttcttgcttg attgctttcg |
| 40381 | tgctttcttg ctttcttgtt ttctttcttt cttttgtttc tttctttctt gcttccttgt |
| 40441 | tttcttgctt tcttgcttgc ttgctttcgt gctttcttgt tttcttgctt tctttctttt |
| 40501 | gtttctttct tgcttgcttt cttgcttcct tgttttcttg ctttcttgct tgcttgcttt |
| 40561 | cgtgctttct ttcttgcttt cttttctttc tttcttttct ttttctttct ttcttgcttt |
| 40621 | cttttctttc atcatcatct ttctttcttt cctttctttc tttctttctt tctatctttc |
| 40681 | tttctttctt tctttctttc tttctttctt tctttctgtt tcgtcctttt gagacagagt |
| 40741 | ttcactcttg tttccacggc tagagtgcaa tggcgcgatc ttggctcacc gcaccttccg |
| 40801 | cctcccgggt tcgagcgctt ctcctgcctc cagcctcccg attagcgggg attgacaggg |
| 40861 | aggcaccccc acgcctggct tggctgatgt ttgtgttttt agtaggcacg ccgtgtctct |
| 40921 | ccatgttgct caggctggtc tccaactccc gacctcctgt gatgcgccca cctcggcctc |
| 40981 | tcgaagtgct gggatgacgg gcgtgacgac cgtgcccggc ctgttgactc atttcgcttt |
| 41041 | tttatttctt tcgtttccac gcgtttactt atatgtatta atgtaaacgt ttctgtacgc |
| 41101 | ttatatgcaa acaacgacaa cgtgtatctc tgcattgaat actcttgcgt atggtaaata |
| 41161 | cgtatcggtt gtatggaaat agacttctgt atgatagatg taggtgtctg tgttatacaa |
| 41221 | ataaatacac atcgctctat aaagaaggga tcgtcgataa agacgtttat tttacgtatg |
| 41281 | aaaagcgtcg tatttatgtg tgtaaatgaa ccgagcgtac gtagttatct ctgttttctt |
| 41341 | tcttcctctc cttcgtgttt ttcttccttc ctttcttcct ttctctcctt ctttaggttt |
| 41401 | ttcttcctct cttcctttcc ttctttctct ctttctgtcc ttttttcctt cgtgctttat |
| 41461 | ttctctttcg ttccctgtgt ttccttcttt tttctttcct ctctgtttct ttttcccttc |
| 41521 | tttccttcgt ttctttcctc attctttctc tctttttcgt tgtttctttc cttcccgtct |
| 41581 | gtcttttaaa aaattggagt gtttcagaag tttactttgt gtatctacgt tttctaaatt |
| 41641 | gtctctcttt tctccatttt cttcctccct ccctccctcc ctccctgctc ccttccctcc |
| 41701 | ctccttccct ttcgccatct gtctcttttc cccactcccc tccccccgtc tgtctctgcg |
| 41761 | tggattccgg aagagcctac cgattctgcc tctccgtgtg tctgcagcga ccccgcgacc |
| 41821 | gagtccttgt gtgttctttc tccctccctc cctccctccc tccctccctc cctccctgct |
| 41881 | tccgagaggc atctccagag accgcgccgt gggttgtctt ctgactctgt cgcggtcgag |
| 41941 | gcagagacgc gttttgggca ccgtttgtgt ggggttgggg cagaggggct gcgttttcgg |
| 42001 | cctcgggaag agcttctcga ctcacggttt cgctttcgcg gtccacgggc cgccctgcca |
| 42061 | gccggatctg tctcgctgac gtccgcggcg gttgtcgggc tccatctggc ggccgctttg |
| 42121 | agatcgtgct ctcggcttcc ggagctgcgg tggcagctgc cgagggaggg gaccgtcccc |
| 42181 | gctgtgagct aggcagagct ccggaaagcc cgcggtcgtc agcccggctg gcccggtggc |
| 42241 | gccagagctg tggccggtcg cttgtgagtc acagctctgg cgtgcaggtt tatgtggggg |
| 42301 | agaggctgtc gctgcgcttc tgggcccgcg gcgggcgtgg ggctgcccgg gccggtcgac |
| 42361 | cagcgcgccg tagctcccga ggcccgagcc gcgacccggc ggacccgccg cgcgtggcgg |
| 42421 | aggctgggga cgcccttccc ggcccggtcg cggtccgctc atcctggccg tctgaggcgg |
| 42481 | cggccgaatt cgtttccgag atccccgtgg ggagccgggg accgtcccgc ccccgtcccc |
| 42541 | cgggtgccgg ggagcggtcc ccgggccggg ccgcggtccc tctgccgcga tcctttctgg |
| 42601 | cgagtccccg tggccagtcg gagagcgctc cctgagccgg tgcggcccga gaggtcgcgc |
| 42661 | tggccggcct tcggtccctc gtgtgtcccg gtcgtaggag gggccggccg aaaatgcttc |
| 42721 | cggctcccgc tctggagaca cgggccggcc cctgcgtgtg gccagggcgg ccgggagggc |
| 42781 | tccccggccc ggcgctgtcc ccgcgtgtgt ccttgggttg accagaggga ccccgggcgc |
| 42841 | tccgtgtgtg gctgcgatgg tggcgttttt ggggacaggt gtccgtgtcc gtgtcgcgcg |
| 42901 | tcgcctgggc cggcggcgtg gtcggtgacg cgacctcccg gccccggggg aggtatatct |
| 42961 | ttcgctccga gtcggcaatt ttgggccgcc gggttatat |

Quadruplex structures for other nucleic acids having sequences derived from human ribosomal DNA, template (T) and non-template (NT) strands are tested. For nucleotide sequences from the NT strand, the number in the identifier delineates the 5′ nucleotide of the oligonucleotide and is the position in SEQ ID NO: 1 less one nucleotide (e.g., the nucleotide sequence of oligonucleotide 13079NT spans sixteen (16) nucleotides in SEQ ID NO: 1 beginning at position 13080 in SEQ ID NO: 1). For nucleotide sequences from the T strand, the number in the identifier defines the 3′ nucleotide of the reverse complement oligonucleotide derived from the position in SEQ ID NO: 1 less one nucleotide (e.g., the nucleotide sequence of 10110T is the reverse complement of a seventeen (17) nucleotide span in SEQ ID NO: 1, with the 3′ terminus of the oligonucleotide defined at position 10111 in SEQ ID NO: 1). Spectra characteristic of parallel, mixed parallel, antiparallel (with mixed parallel characteristics) and complex intramolecular quadruplex structures were observed. Quadruplex conformation determinations are summarized in the following table.

| Nucleic | | |
|---|---|---|
| acid | | |
| identifier | Conformation | Nucleotide Sequence |
| 10110T | Parallel | GGGGGGGGGGGCGGGGG |
| 13079NT | Parallel | GGGGTGGGGGGGAGGG |
| 6960NT | Mixed | GGGTGGCGGGGGGGAGAGGGGGG |
| 6534NT | Mixed | GGGCGGGGGGGGCGGGGGG |
| 1196NT | Mixed | GGGTGGACGGGGGGGCCTGGTGGGG |
| 2957NT | Mixed | GGGTCGGGGGGTGGGGCCCGGGCCG |
| | | GGG |
| 5700NT | Mixed | GGGAGGGAGACGGGGGGG |
| 8511NT | Mixed | GGGGGTGGGCGGGCGGGGCCGGGGG |
| | | TGGG |
| 6183NT | Mixed | GGGTCGGGGGCGGTGGTGGGCCCGCG |
| | | GGGG |
| 11028NT | Mixed | GGGGCGCGGCGGGGGGAGAAGGGTC |
| | | GGGGCGGCAGGGG |
| 6374NT | Mixed | GGGGGCGGGAACCCCCGGGCGCCTGT |
| | | GGG |
| 7733T | Mixed | GGGAGGGGCACGGGCCGGGGGCGGGA |
| | | CGGG |
| 7253NT | Mixed | GGGTCCGGAAGGGGAAGGGTGCCGG |
| | | CGGGGAGAGAGGGTCGGGGG |
| 13173NT | Mixed | GGGCCGGGACGGGGTCCGGGG |
| 6914T | Mixed | GGGCCCGCGGGGGGAGGGGGAAGGG |
| | | GCGGG |
| 8749NT | Antiparallel | GGGAGGGCGCGCGGGTCGGGG |
| 10816NT | Antiparallel | GGGCTGGGTCGGTCGGGCTGGGG |
| 8762NT | Complex | CGGAGGGCGCGCGGGTCGGGGCGG |
| | | CGGCGGCGGCGGCGGTGGCGGCGG |
| | | CGGCGGGGGCGGCGGG |

### Example 4

### Effects of Ribosomal Nucleic Acid Interacting Molecules on Nucleolin/Nucleic Acid Interactions

The following assays can be used to assess the effects of compounds on interactions between nucleolin and nucleic acid ligands capable of forming quadruplex (QP) and hairpin (HP) secondary structures. Nucleic acid ligands tested were a cMyc QP DNA having nucleotide sequence 5′-TGGGGAGGGTGGGGAGGGTGGGGAAGG-3′ and a HP pre-rRNA region to which nucleolin binds, having the sequence 5′-GGCCGAAAUCCCGAAGUAGGCC-3′. In the assays, recombinant nucleolin (˜250 nM), which has been fused to maltose binding protein, and has the sequence under accession number NM_{—}005381 without the N-terminal acidic stretches domain, is incubated with each of the two ³²P-labeled nucleic acid ligands (10 or 250 nM). Nucleolin and the nucleic acid ligand are incubated in the presence or absence of a test compound 7 in an incubation buffer (12.5 mM Tris, pH 7.6, 60 mM KCl, 1 mM MgCl₂, 0.1 mM EDTA, 1 mM DTT, 5% glycerol, 0.1 mg/ml BSA) for 30 minutes at room temperature.

The resulting complexes are separated on a 6% DNA retardation gel using 0.5×TBE with 20 mM KCl as a running buffer. The assay also can be conducted using nucleic acid ligands derived from human ribosomal DNA, whereby one can identify a compound that selectively modulates formation of a nucleolin/nucleic acid complex that depends on the conformation of the nucleic acid. Sequences of suitable nucleic acids are shown in the preceding example. The table directly below shows for each nucleic acid ligand the relative affinity for nucleolin. A “+” represents the weakest nucleolin affinity and a “++++” represents the strongest nucleolin affinity. The table also shows the conformation of the intramolecular quadruplex structure formed by the nucleic acid ligand determined by circular dichroism, as described above. RND27 is a single-stranded nucleic acid having a random sequence that does not form a quadruplex structure. Using nucleic acids such as these having known conformational properties, one can identify a compound such as the compounds described herein that selectively interferes with binding of nucleolin to a particular quadruplex structure.

| Nucleic acid ligand | Conformation | Affinity for Nucleolin |
|---|---|---|
| 1196NT | Mixed | + |
| 2957NT | Mixed | +++ |
| 6183NT | Mixed | + |
| 6374NT | Mixed | − |
| 6534NT | Parallel | +++ |
| 6960NT | Parallel | +++ |
| 7253NT | Mixed | +++ |
| 7733T | Mixed | + |
| 8511NT | Mixed | ++++ |
| 8749NT | Antiparallel | + |
| 8762NT | Complex | ++++ |
| 10816NT | Antiparallel | − |
| 11028NT | Mixed | + |
| 13079NT | Parallel | ++ |
| 13137NT | Mixed | ++ |
| RND27 | Single-stranded | − |

### Example 5

### Inhibition of Protein Kinases

Compounds can also be tested for activity in protein kinase inhibition assays as described herein. All substrates are dissolved and diluted to working stocks in de-ionised water, apart from histone H1 (10× working stock in 20 mM MOPS pH 7.0), PDKtide (10× working stock in 50 mM Tris pH 7.0) ATF2 (which is typically stored at a 20× working stock in 50 mM Tris pH 7.5, 150 mM NaCl, 0.1 mM EGTA, 0.03% Brij-35, 50% glycerol, 1 mM benzamidine, 0.2 mM PMSF and 0.1% R-mercaptoethanol), KKLNRTLSFAEPG and RRRLSFAEPG (50 mM HEPES pH 7.4) and GGEEEEYFELVKKKK (20 mM MOPS pH 7.0). All kinases are pre-diluted to a 10× working concentration prior to addition into the assay. The composition of the dilution buffer for each kinase is detailed below.

1. Blk, c-RAF, CSK, IGF-1R, IR, Lyn, MAPK1, MAPK2, MKK4, MKK6, MKK70, SAPK2a, SAPK2b, SAPK3, SAPK4, Syk, ZAP-70: 50 mM Tris pH 7.5, 0.1 mM EGTA, 0.1 mM Na3VO4, 0.1% beta-mercaptoethanol, 1 mg/ml BSA.

2. JNK1a1, JNK2a2, JNK3, PRK2, ROCK-II: 50 mM Tris pH 7.5, 0.1 mM EGTA, 0.1% beta-mercaptoethanol, 1 mg/ml BSA.

3. PDK1: 50 mM Tris pH 7.5, 0.05% Beta-mercaptoethanol, 1 mg/ml BSA.

4. MEK-1: 25 mM Tris pH 7.5, 0.1 mM EGTA, 0.1% beta-mercaptoethanol, 1 mg/ml BSA.

5. Abl, Abl(T315I), ALK, ALK4, Arg, Ask1, Aurora-A, Axl, Bmx, BRK, BTK, CDK1/cyclinB, CDK2/cyclinA, CDK2/cyclinE, CDK3/cyclinE, CDK5/p25, CDK5/p35, CDK6/cyclinD3, CDK7/cyclinH/MAT1, CHK1, CHK2, CK1, CKIS, cKit, cKit (D816V), cSRC, DDR2, EGFR, EGFR (L858R), EGFR (L861Q), EphA2, EphA3, EphA4, EphA5, EphB2, EphB3, EphB4, ErbB4, Fer, Fes, FGFR1, FGFR2, FGFR3, FGFR4, Fgr, Flt1, Flt3, Flt3 (D835Y), Fms, Fyn, GSK3a, GSK30, Hck, HIPK2, IKKa, IKKO, IRAK4, IRR, JAK2, JAK3, KDR, Lck, MAPKAP-K2, MAPKAP-K3, Met, MINK, MLCK, MRCKP, MSK1, MSK2, MST1, MST2, MuSK, NEK2, NEK6, Nek7, p70S6K, PAK2, PAK4, PAK6, PAR-1Ba, PDGFRa, PDGFRO, Pim-1, PKA, PKBa, PKBP, PKBy, PKC6, PKCQ, PKG10, Plk3, Pyk2, Ret, RIPK2, Rse, ROCK-I, Ron, Ros, Rsk1, Rsk2, Rsk3, SGK, SGK2, SGK3, Snk, TAK1, TBK1, Tie2, TrkA, TrkB, TSSK2, Yes, ZIPK: 20 mM MOPS pH 7.0, 1 mM EDTA, 0.1% Beta-mercaptoethanol, 0.01% Brij-35, 5% glycerol, 1 mg/ml BSA.

6. CK2: 20 mM HEPES pH 7.6, 0.15 M NaCl, 0.1 mM EGTA, 5 mM DTT, 0.1% Triton X-100, 50% glycerol.

7. CaMKII, CaMKIV: 40 mM HEPES pH 7.4, 1 mg/ml BSA.

8. PKCa, PKCRI, PKCRII, PKCy, PKCS, PKC6, PKCYI, PKCL, PKCμ, PKD2: 20 mM HEPES pH 7.4, 0.03% Triton X-100.

9. PRAK: Beta-mercaptoethanol, 0.1 mM EGTA, 1 mg/ml BSA.

10. AMPK: 50 mM Na R-glycerophosphate pH 7.0, 0.1%. Protein kinase assays for a variety of kinases are conducted as follows:

Abl (h)

In a final reaction volume of 25 μl, Abl (h) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Abl (T315I) (h)

In a final reaction volume of 25 μl, Abl (T315I) (h) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Abl (m)

In a final reaction volume of 25 μl, Abl (m) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in meth\anol prior to drying and scintillation counting.

ALK (h)

In a final reaction volume of 25 μl, ALK (h) (5-10 mU) is incubated with 8 mM MOPS pH 7.0, 0.2 mM EDTA, 250 μM KKKSPGEYVNIEFG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting. ALK4 (h)

In a final reaction volume of 25 μl, ALK4 (h) (5-10 mU) is incubated with 8 mM MOPS pH 7.0, 0.2 mM EDTA, 2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

AMPK (r)

In a final reaction volume of 25 μl, AMPK (r) (5-10 mU) is incubated with 32 mM HEPES pH 7.4, 0.65 mM DTT, 0.012% Brij-35, 200 μM AMP, 200 μM AMARAASAAALARRR, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Arg (h)

In a final reaction volume of 25 μl, Arg (h) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Arg (m)

In a final reaction volume of 25 μl, Arg (m) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ASK1 (h)

In a final reaction volume of 25 μl, ASK1 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Aurora-A (h)

In a final reaction volume of 25 μl, Aurora-A (h) (5-10 mU) is incubated with 8 mM MOPS pH 7.0, 0.2 mM EDTA, 200 μM LRRASLG (Kemptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

Axl (h)

In a final reaction volume of 25 μl, Axl (h) (5-10 mU) is incubated with 8 mM MOPS pH 7.0, 0.2 mM EDTA, 250 μM KKSRGDYMTMQIG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Blk (m)

In a final reaction volume of 25 μl, Blk (m) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Bmx (h)

In a final reaction volume of 25 μl, Bmx (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

BRK (h)

In a final reaction volume of 25 μl, BRK (h) (5-10 mU) is incubated with 8 mM MOPS pH 7.0, 0.2 mM EDTA, 5 mM MnCl2, 0.1 mg/ml poly (Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

BTK (h)

In a final reaction volume of 25 μl, BTK (h) (5-10 mU) is incubated with 8 mM MOPS pH 7.0, 0.2 mM EDTA, 250 μM KVEKIGEGTYGVVYK (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CaMKII (r)

In a final reaction volume of 25 μl, CaMKII (r) (5-10 mU) is incubated with 40 mM HEPES pH 7.4, 5 mM CaCl2, 30 μg/ml calmodulin, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CaMKIV (h)

In a final reaction volume of 25 μl, CaMKIV (h) (5-10 mU) is incubated with 40 mM HEPES pH 7.4, 5 mM CaCl2, 30 μg/ml calmodulin, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK1/cyclinB (h)

In a final reaction volume of 25 μl, CDK1/cyclinB (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK2/cyclinA (h)

In a final reaction volume of 25 μl, CDK2/cyclinA (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK2/cyclinE (h)

In a final reaction volume of 25 μl, CDK2/cyclinE (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK3/cyclinE (h)

In a final reaction volume of 25 μl, CDK3/cyclinE (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK5/p25 (h)

In a final reaction volume of 25 μl, CDK5/p25 (h) (5-10 mU) is incubated with 8 mM MOPS pH 7.0, 0.2 mM EDTA, 0.1 mg/ml histone H1, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK5/p35 (h)

In a final reaction volume of 25 μl, CDK5/p35 (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK6/cyclinD3 (h)

In a final reaction volume of 25 μl, CDK6/cyclinD3 (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK7/cyclinH/MAT1 (h)

In a final reaction volume of 25 μl, CDK7/cyclinH/MAT1 (h) (5-10 mU) is incubated with 8 mM MOPS pH 7.0, 0.2 mM EDTA, 500 μM peptide, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CHK1 (h)

In a final reaction volume of 25 μl, CHK1 (h) (5-10 mU) is incubated with 8 mM MOPS pH 7.0, 0.2 mM EDTA, 200 μM KKKVSRSGLYRSPSMPENLNRPR, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CHK2 (h)

In a final reaction volume of 25 μl, CHK2 (h) (5-10 mU) is incubated with 8 mM MOPS pH 7.0, 0.2 mM EDTA, 200 μM KKKVSRSGLYRSPSMPENLNRPR, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CK1 (y)

In a final reaction volume of 25 μl, CK1 (y) (5-10 mU) is incubated with 8 mM MOPS pH 7.0, 0.2 mM EDTA, 200 μM KRRRALS(p)VASLPGL, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CK1S (h)

In a final reaction volume of 25 μl, CK1S (h) (5-10 mU) is incubated with 8 mM MOPS pH 7.0, 0.2 mM EDTA, 200 μM KRRRALS(p)VASLPGL, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CK2 (h)

In a final reaction volume of 25 μl, CK2 (h) (5-10 mU) is incubated with 20 mM HEPES pH 7.6, 0.15 M NaCl, 0.1 mM EDTA, 5 mM DTT, 0.1% Triton X-100, 165 μM RRRDDDSDDD, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

cKit (h)

In a final reaction volume of 25 μl, cKit (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

cKit (D816V) (h)

In a final reaction volume of 25 μl, cKit (D816V) (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

c-RAF (h)

In a final reaction volume of 25 μl, c-RAF (h) (5-10 mU) is incubated with 25 mM Tris pH 7.5, 0.02 mM EGTA, 0.66 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CSK (h)

In a final reaction volume of 25 μl, CSK (h) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

cSRC (h)

In a final reaction volume of 25 μl, cSRC (h) (5-10 mU) is incubated with 8 mM MOPS pH 7.0, 0.2 mM EDTA, 250 μM KVEKIGEGTYGVVYK (Cdc2 peptide), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

DDR2 (h)

In a final reaction volume of 25 μl, DDR2 (h) (5-10 mU) is incubated with 8 mM MOPS pH 7.0, 0.2 mM EDTA, 250 μM KKSRGDYMTMQIG, 10 mM MnCl2, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

EGFR (h)

In a final reaction volume of 25 μl, EGFR (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EGFR (L858R) (h)

In a final reaction volume of 25 μl, EGFR (L858R) (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EGFR (L861Q) (h)

In a final reaction volume of 25 μl, EGFR (L861Q) (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphA2 (h)

In a final reaction volume of 25 μl, EphA2 (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphA3 (h)

In a final reaction volume of 25 μl, EphA3 (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphA4 (h)

In a final reaction volume of 25 μl, EphA4 (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphA5 (h)

In a final reaction volume of 25 μl, EphA5 (h) (5-10 mU) is incubated with 8 mM MOPS pH 7.0, 0.2 mM EDTA, 2.5 mM MnCl2, 0.1 mg/ml poly (Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphB2 (h)

In a final reaction volume of 25 μl, EphB2 (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphB3 (h)

In a final reaction volume of 25 μl, EphB3 (h) (5-10 mU) is incubated with 8 mM MOPS pH 7.0, 0.2 mM EDTA, 10 mM MnCl2, 0.1 mg/ml poly (Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphB4 (h)

In a final reaction volume of 25 μl, EphB4 (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

ErbB4 (h)

In a final reaction volume of 25 μl, ErbB4 (h) (5-10 mU) is incubated with 8 mM MOPS pH 7.0, 0.2 mM EDTA, 2.5 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Fer (h)

In a final reaction volume of 25 μl, Fer (h) (5-10 mU) is incubated with 8 mM MOPS pH 7.0, 0.2 mM EDTA, 1 mM MnCl2, 250 μM KKKSPGEYVNIEFG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Fes (h)

In a final reaction volume of 25 μl, Fes (h) (5-10 mU) is incubated with 8 mM MOPS pH 7.0, 0.2 mM EDTA, 0.1 mg/ml poly(Glu, Tyr) 4:1, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

FGFR1 (h)

In a final reaction volume of 25 μl, FGFR1 (h) (5-10 mU) is incubated with 8 mM MOPS pH 7.0, 0.2 mM EDTA, 250 μM KKKSPGEYVNIEFG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

FGFR2 (h)

In a final reaction volume of 25 μl, FGFR2 (h) (5-10 mU) is incubated with 8 mM MOPS pH 7.0, 0.2 mM EDTA, 2.5 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

FGFR3 (h)

In a final reaction volume of 25 μl, FGFR3 (h) (5-10 mU) is incubated with 8 mM MOPS pH 7.0, 0.2 mM EDTA, 0.1 mg/ml poly(Glu, Tyr) 4:1, 10 mM MnCl2, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

FGFR4 (h)

In a final reaction volume of 25 μl, FGFR4 (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Fgr (h)

In a final reaction volume of 25 μl, Fgr (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Flt1 (h)

In a final reaction volume of 25 μl, Flt1 (h) (5-10 mU) is incubated with 8 mM MOPS pH 7.0, 0.2 mM EDTA, 250 μM KKKSPGEYVNIEFG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Flt3 (h)

In a final reaction volume of 25 μl, Flt3 (h) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Flt3 (D835Y) (h)

In a final reaction volume of 25 μl, Flt3 (D835Y) (h) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Fms (h)

In a final reaction volume of 25 μl, Fms (h) (5-10 mU) is incubated with 8 mM MOPS pH 7.0, 0.2 mM EDTA, 250 μM KKKSPGEYVNIEFG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Fyn (h)

In a final reaction volume of 25 μl, Fyn (h) (5-10 mU) is incubated with 50 mM Tris pH 7.5, 0.1 mM EGTA, 0.1 mM Na3VO4, 250 μM KVEKIGEGTYGVVYK (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

GSK3a (h)

In a final reaction volume of 25 μl, GSK3a (h) (5-10 mU) is incubated with 8 mM MOPS pH 7.0, 0.2 mM EDTA, 20 μM YRRAAVPPSPSLSRHSSPHQS(p)EDEEE (phospho GS2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

GSK3P (h)

In a final reaction volume of 25 μl, GSK30 (h) (5-10 mU) is incubated with 8 mM MOPS pH 7.0, 0.2 mM EDTA, 20 μM YRRAAVPPSPSLSRHSSPHQS(p)EDEEE (phospho GS2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

Hck (h)

In a final reaction volume of 25 μl, Hck (h) (5-10 mU) is incubated with 8 mM MOPS pH 7.0, 0.2 mM EDTA, 250 μM KVEKIGEGTYGVVYK (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

HIPK2 (h)

In a final reaction volume of 25 μl, HIPK2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IGF-1R (h)

In a final reaction volume of 25 μl, IGF-1R (h) (5-10 mU) is incubated with 50 mM Tris pH 7.5, 0.1 mM EGTA, 0.1 mM Na3VO4, 0.1% R-mercaptoethanol, 250 μM KKKSPGEYVNIEFG, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IKKa (h)

In a final reaction volume of 25 μl, IKKa (h) (5-10 mU) is incubated with 8 mM MOPS pH 7.0, 0.2 mM EDTA, 200 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IKKP (h)

In a final reaction volume of 25 μl, IKKP (h) (5-10 mU) is incubated with 8 mM MOPS pH 7.0, 0.2 mM EDTA, 100 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IR (h)

In a final reaction volume of 25 μl, IR (h) (5-10 mU) is incubated with 50 mM Tris pH 7.5, 0.1 mM EGTA, 0.1 mM Na3VO4, 0.1% R-mercaptoethanol, 250 μM KKSRGDYMTMQIG, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IRAK4 (h)

In a final reaction volume of 25 μl, IRAK4 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IRR (h)

In a final reaction volume of 25 μl, IRR (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JAK2 (h)

In a final reaction volume of 25 μl, JAK2 (h) (5-10 mU) is incubated with 8 mM MOPS pH 7.0, 0.2 mM EDTA, 100 μM KTFCGTPEYLAPEVRREPRILSEEEQEMFRDFDYIADWC, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JAK3 (h)

In a final reaction volume of 25 μl, JAK3 (h) (5-10 mU) is incubated with 8 mM MOPS pH 7.0, 0.2 mM EDTA, 500 μM GGEEEEYFELVKKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JNK1a1 (h)

In a final reaction volume of 25 μl, JNK1a1 (h) (5-10 mU) is incubated with 50 mM Tris pH 7.5, 0.1 mM EGTA, 0.1% R-mercaptoethanol, 3 μM ATF2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JNK2a2 (h)

In a final reaction volume of 25 μl, JNK2a2 (h) (5-10 mU) is incubated with 50 mM Tris pH 7.5, 0.1 mM EGTA, 0.1% R-mercaptoethanol, 3 μM ATF2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JNK3 (h)

In a final reaction volume of 25 μl, JNK3 (h) (5-10 mU) is incubated with 50 mM Tris pH 7.5, 0.1 mM EGTA, 0.1% R-mercaptoethanol, 250 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

KDR (h)

In a final reaction volume of 25 μl, KDR (h) (5-10 mU) is incubated with 8 mM MOPS pH 7.0, 0.2 mM EDTA, 0.33 mg/ml myelin basic protein, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Lck (h)

In a final reaction volume of 25 μl, Lck (h) (5-10 mU) is incubated with 50 mM Tris pH 7.5, 0.1 mM EGTA, 0.1 mM Na3VO4, 250 μM KVEKIGEGTYGVVYK (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Lyn (h)

In a final reaction volume of 25 μl, Lyn (h) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Lyn (m)

In a final reaction volume of 25 μl, Lyn (m) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

MAPK1 (h)

In a final reaction volume of 25 μl, MAPK1 (h) (5-10 mU) is incubated with 25 mM Tris pH 7.5, 0.02 mM EGTA, 250 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MAPK2 (h)

In a final reaction volume of 25 μl, MAPK2 (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

P,-MAPK2 (m)

In a final reaction volume of 25 μl, MAPK2 (m) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MAPKAP-K2 (h)

In a final reaction volume of 25 μl, MAPKAP-K2 (h) (5-10 mU) is incubated with 50 mM Na R-glycerophosphate pH 7.5, 0.1 mM EGTA, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MAPKAP-K3 (h)

In a final reaction volume of 25 μl, MAPKAP-K3 (h) (5-10 mU) is incubated with 50 mM Na R-glycerophosphate pH 7.5, 0.1 mM EGTA, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MEK1 (h)

In a final reaction volume of 25 μl, MEK1 (h) (1-5 mU) is incubated with 50 mM Tris pH 7.5, 0.2 mM EGTA, 0.1% R-mercaptoethanol, 0.01% Brij-35, 1 μM inactive MAPK2 (m), 10 mM MgAcetate and cold ATP (concentration as required). The reaction is initiated by the addition of the MgATP. After incubation for 40 minutes at room temperature, 5 μl of this incubation mix is used to initiate a MAPK2 (m) assay, which is described on page 12 of this book.

Met (h)

In a final reaction volume of 25 μl, Met (h) (5-10 mU) is incubated with 8 mM MOPS pH 7.0, 0.2 mM EDTA, 250 μM KKKSPGEYVNIEFG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MINK (h)

In a final reaction volume of 25 μl, MINK (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MKK4 (m)

In a final reaction volume of 25 μl, MKK4 (m) (1-5 mU) is incubated with 50 mM Tris pH 7.5, 0.1 mM EGTA, 0.1% R-mercaptoethanol, 0.1 mM Na3VO4, 2 μM inactive JNK1a1 (h), 10 mM MgAcetate and cold ATP (concentration as required). The reaction is initiated by the addition of the MgATP. After incubation for 40 minutes at room temperature, 5 μl of this incubation mix is used to initiate a JNK1a1 (h) assay, which is exactly as described on page 11 of this book except that ATF2 is replaced with 250 μM peptide.

MKK6 (h)

In a final reaction volume of 25 μl, MKK6 (h) (1-5 mU) is incubated with 50 mM Tris pH 7.5, 0.1 mM EGTA, 0.1% R-mercaptoethanol, 0.1 mM Na3VO4, 1 mg/ml BSA, 1 μM inactive SAPK2a (h), 10 mM MgAcetate and cold ATP (concentration as required). The reaction is initiated by the addition of the MgATP. After incubation for 40 minutes at room temperature, 5 μl of this incubation mix is used to initiate a SAPK2a (h) assay, which is described on page 18 of this book.

MKK7P (h)

In a final reaction volume of 25 μl, MKK70 (h) (1-5 mU) is incubated with 50 mM Tris pH 7.5, 0.1 mM EGTA, 0.1% R-mercaptoethanol, 0.1 mM Na3VO4, 2 μM inactive JNK1a1 (h), 10 mM MgAcetate and cold ATP (concentration as required). The reaction is initiated by the addition of the MgATP. After incubation for 40 minutes at room temperature, 5 μl of this incubation mix is used to initiate a JNK1a1 (h) assay, which is exactly as described on page 11 of this book except that ATF2 is replaced with 250 μM peptide.

MLCK (h)

In a final reaction volume of 25 μl, MLCK (h) (5-10 mU) is incubated with 8 mM MOPS pH 7.0, 0.2 mM EDTA, 0.5 mM CaCl2, 16 μg/ml calmodulin, 250 μM KKLNRTLSFAEPG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MRCKP (h)

In a final reaction volume of 25 μl, MRCKP (h) (5-10 mU) is incubated with 8 mM MOPS pH 7.0, 0.2 mM EDTA, 100 μM KKRNRTLTV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MSK1 (h)

In a final reaction volume of 25 μl, MSK1 (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MSK2 (h)

In a final reaction volume of 25 μl, MSK2 (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MST1 (h)

In a final reaction volume of 25 μl, MST1 (h) (5-10 mU) is incubated with 8 mM MOPS pH 7.0, 0.2 mM EDTA, 250 μM KKSRGDYMTMQIG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MST2 (h)

In a final reaction volume of 25 μl, MST2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MuSK (h)

In a final reaction volume of 25 μl, MuSK (h) (5-10 mU) is incubated with 8 mM MOPS pH 7.0, 0.2 mM EDTA, 5 mM MnCl2, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

NEK2 (h)

In a final reaction volume of 25 μl, NEK2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

NEK6 (h)

In a final reaction volume of 25 μl, NEK6 (h) (5-10 mU) is incubated with 8 mM MOPS pH 7.0, 0.2 mM EDTA, 300 μM FLAKSFGSPNRAYKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

NEK7 (h)

In a final reaction volume of 25 μl, NEK7 (h) (5-10 mU) is incubated with 8 mM MOPS pH 7.0, 0.2 mM EDTA, 300 μM FLAKSFGSPNRAYKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAK2 (h)

In a final reaction volume of 25 μl, PAK2 (h) (5-10 mU) is incubated with 8 mM MOPS pH 7.0, 0.2 mM EDTA, 30 μM KEAKEKRQEQIAKRRRLSSLRASTSKSGGSQK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAK4 (h)

In a final reaction volume of 25 μl, PAK4 (h) (5-10 mU) is incubated with 8 mM MOPS pH 7.0, 0.2 mM EDTA, 0.8 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAK6 (h)

In a final reaction volume of 25 μl, PAK6 (h) (5-10 mU) is incubated with 8 mM MOPS pH 7.0, 0.2 mM EDTA, 200 μM RRRLSFAEPG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAR-1Ba (h)

In a final reaction volume of 25 μl, PAR-1Ba (h) (5-10 mU) is incubated with 8 mM MOPS pH 7.0, 0.2 mM EDTA, 100 μM KKKVSRSGLYRSPSMPENLNRPR, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PDGFRa (h)

In a final reaction volume of 25 μl, PDGFRa (h) (5-10 mU) is incubated with 8 mM MOPS pH 7.0, 0.2 mM EDTA, 0.1 mg/ml poly(Glu, Tyr) 4:1, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

PDGFRP (h)

In a final reaction volume of 25 μl, PDGFRP (h) (5-10 mU) is incubated with 8 mM MOPS pH 7.0, 0.2 mM EDTA, 0.1 mg/ml poly(Glu, Tyr) 4:1, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

PDK1 (h)

In a final reaction volume of 25 μl, PDK1 (h) (5-10 mU) is incubated with 50 mM Tris pH 7.5, 100 μM KTFCGTPEYLAPEVRREPRILSEEEQEMFRDFDYIADWC (PDKtide), 0.1% R-mercaptoethanol, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PI3Ky (h) [Non-radioactive assay]

In a final reaction volume of 20 μl, PI3Ky (h) is incubated in assay buffer containing 10 μM phosphatidylinositol-4,5-bisphosphate and MgATP (concentration as required). The reaction is initiated by the addition of the MgATP mix. After incubation for 30 minutes at room temperature, the reaction is stopped by the addition of 5 μl of stop solution containing EDTA and biotinylated phosphatidylinositol-3,4,5-trisphosphate. Finally, 5 μl of detection buffer is added, which contains europium-labelled anti-GST monoclonal antibody, GST-tagged GRP1 PH domain and streptavidin-allophycocyanin. The plate is then read in time-resolved fluorescence mode and the homogenous time-resolved fluorescence (HTRF®)* signal is determined according to the formula HTRF®=10000×(Em665 nm/Em620 nm).

Pim-1 (h)

In a final reaction volume of 25 μl, Pim-1 (h) (5-10 mU) is incubated with 8 mM MOPS pH 7.0, 0.2 mM EDTA, 100 μM KKRNRTLTV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKA (h)

In a final reaction volume of 25 μl, PKA (h) (5-10 mU) is incubated with 8 mM MOPS pH 7.0, 0.2 mM EDTA, 30 μM LRRASLG (Kemptide), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

PKA (b)

In a final reaction volume of 25 μl, PKA (b) (5-10 mU) is incubated with 8 mM MOPS pH 7.0, 0.2 mM EDTA, 30 μM LRRASLG (Kemptide), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

PKBa (h)

In a final reaction volume of 25 μl, PKBa (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKBP (h)

In a final reaction volume of 25 μl, PKBP (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKBy (h)

In a final reaction volume of 25 μl, PKBy (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCa (h)

In a final reaction volume of 25 μl, PKCa (h) (5-10 mU) is incubated with 20 mM HEPES pH 7.4, 0.03% Triton X-100, 0.1 mM, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCPI (h)

In a final reaction volume of 25 μl, PKCRI (h) (5-10 mU) is incubated with 20 mM HEPES pH 7.4, 0.03% Triton X-100, 0.1 mM CaCl2, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCPII (h)

In a final reaction volume of 25 μl, PKCRII (h) (5-10 mU) is incubated with 20 mM HEPES pH 7.4, 0.03% Triton X-100, 0.1 mM, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCy (h)

In a final reaction volume of 25 μl, PKCy (h) (5-10 mU) is incubated with 20 mM HEPES pH 7.4, 0.03% Triton X-100, 0.1 mM, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCS (h)

In a final reaction volume of 25 μl, PKCS (h) (5-10 mU) is incubated with 20 mM HEPES pH 7.4, 0.03% Triton X-100, 0.1 mg/ml phosphatidylserine, 10 μg/ml diacylglycerol, 50 μM ERMRPRKRQGSVRRRV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCS (h)

In a final reaction volume of 25 μl, PKC6 (h) (5-10 mU) is incubated with 20 mM HEPES pH 7.4, 0.03% Triton X-100, 0.1 mg/ml phosphatidylserine, 10 μg/ml diacylglycerol, 50 μM ERMRPRKRQGSVRRRV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCYI (h)

In a final reaction volume of 25 μl, PKCYj (h) (5-10 mU) is incubated with 20 mM HEPES pH 7.4, 0.03% Triton X-100, 0.1 mM CaCl2, 0.1 mg/ml phosphatidylserine, 10 μg/ml diacylglycerol, 50 μM ERMRPRKRQGSVRRRV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCL (h)

In a final reaction volume of 25 μl, PKCL (h) (5-10 mU) is incubated with 20 mM HEPES pH 7.4, 0.03% Triton X-100, 50 μM ERMRPRKRQGSVRRRV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCμ (h)

In a final reaction volume of 25 μl, PKCV (h) (5-10 mU) is incubated with 20 mM HEPES pH 7.4, 0.03% Triton X-100, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCe (h)

In a final reaction volume of 25 μl, PKC6 (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCM (h)

In a final reaction volume of 25 μl, PKCQ (h) (5-10 mU) is incubated with 8 mM MOPS pH 7.0, 0.2 mM EDTA, 50 μM ERMRPRKRQGSVRRRV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKD2 (h)

In a final reaction volume of 25 μl, PKD2 (h) (5-10 mU) is incubated with 20 mM HEPES pH 7.4, 0.03% Triton X-100, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKG1P (h)

In a final reaction volume of 25 μl, PKG10 (h) (5-10 mU) is incubated with 8 mM MOPS pH 7.0, 0.2 mM EDTA, 10 μM cGMP, 200 μM RRRLSFAEPG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Plk3 (h)

In a final reaction volume of 25 μl, Plk3 (h) (5-10 mU) is incubated with 8 mM MOPS pH 7.0, 0.2 mM EDTA, 2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PRAK (h)

In a final reaction volume of 25 μl, PRAK (h) (5-10 mU) is incubated with 50 mM Na R-glycerophosphate pH 7.5, 0.1 mM EGTA, 30 μM KKLRRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

PRK2 (h)

In a final reaction volume of 25 μl, PRK2 (h) (5-10 mU) is incubated with 50 mM Tris pH 7.5, 0.1 mM EGTA, 0.1% R-mercaptoethanol, 30 μM AKRRRLSSLRA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Pyk2 (h)

In a final reaction volume of 25 μl, Pyk2 (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

p70S6K (h)

In a final reaction volume of 25 μl, p70S6K (h) (5-10 mU) is incubated with 8 mM MOPS pH 7.0, 0.2 mM EDTA, 100 μM KKRNRTLTV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Ret (h)

In a final reaction volume of 25 μl, Ret (h) (5-10 mU) is incubated with 8 mM MOPS pH 7.0, 0.2 mM EDTA, 250 μM KKKSPGEYVNIEFG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

RIPK2(h)

In a final reaction volume of 25 μl, RIPK2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ROCK-I (h)

In a final reaction volume of 25 μl, ROCK-I (h) (5-10 mU) is incubated with 8 mM MOPS pH 7.0, 0.2 mM EDTA, 30 μM KEAKEKRQEQIAKRRRLSSLRASTSKSGGSQK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ROCK-II (h)

In a final reaction volume of 25 μl, ROCK-II (h) (5-10 mU) is incubated with 50 mM Tris pH 7.5, 0.1 mM EGTA, 30 μM KEAKEKRQEQIAKRRRLSSLRASTSKSGGSQK, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ROCK-II (r)

In a final reaction volume of 25 μl, ROCK-II (r) (5-10 mU) is incubated with 50 mM Tris pH 7.5, 0.1 mM EGTA, 30 μM KEAKEKRQEQIAKRRRLSSLRASTSKSGGSQK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Ron (h)

In a final reaction volume of 25 μl, Ron (h) (5-10 mU) is incubated with 8 mM MOPS pH 7.0, 0.2 mM EDTA, 250 μM KKSRGDYMTMQIG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Ros (h)

In a final reaction volume of 25 μl, Ros (h) (5-10 mU) is incubated with 8 mM MOPS pH 7.0, 0.2 mM EDTA, 10 mM MnCl2, 250 μM KKKSPGEYVNIEFG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rse (h)

In a final reaction volume of 25 μl, Rse (h) (5-10 mU) is incubated with 8 mM MOPS pH 7.0, 0.2 mM EDTA, 250 μM KVEKIGEGTYGVVYK, 1 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk1 (h)

In a final reaction volume of 25 μl, Rsk1 (h) (5-10 mU) is incubated with 8 mM MOPS pH 7.0, 0.2 mM EDTA, 30 μM KKKNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk1 (r)

In a final reaction volume of 25 μl, Rsk1 (r) (5-10 mU) is incubated with 8 mM MOPS pH 7.0, 0.2 mM EDTA, 30 μM KKKNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk2 (h)

In a final reaction volume of 25 μl, Rsk2 (h) (5-10 mU) is incubated with 8 mM MOPS pH 7.0, 0.2 mM EDTA, 30 μM KKKNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk3 (h)

In a final reaction volume of 25 μl, Rsk3 (h) (5-10 mU) is incubated with 8 mM MOPS pH 7.0, 0.2 mM EDTA, 30 μM KKKNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK2a (h)

In a final reaction volume of 25 μl, SAPK2a (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK2b (h)

In a final reaction volume of 25 μl, SAPK2b (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK3 (h)

In a final reaction volume of 25 μl, SAPK3 (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK4 (h)

In a final reaction volume of 25 μl, SAPK4 (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SGK (h)

In a final reaction volume of 25 μl, SGK (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SGK2 (h)

In a final reaction volume of 25 μl, SGK2 (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SGK3 (h)

In a final reaction volume of 25 μl, SGK3 (h) (5-10 mU) is incubated with 8 mM MOPS pH 7.0, 0.2 mM EDTA, 250 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Snk (h)

In a final reaction volume of 25 μl, Snk (h) (5-10 mU) is incubated with 8 mM MOPS pH 7.0, 0.2 mM EDTA, 2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Syk (h)

In a final reaction volume of 25 μl, Syk (h) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

TAK1 (h)

In a final reaction volume of 25 μl, TAK1 (h) (5-10 mU) is incubated with 8 mM MOPS pH 7.0, 0.2 mM EDTA, 2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

TBK1 (h)

In a final reaction volume of 25 μl, TBK1 (h) (5-10 mU) is incubated with 8 mM MOPS pH 7.0, 0.2 mM EDTA, 200 μM KRRRALS(p)VASLPGL, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Tie2 (h)

In a final reaction volume of 25 μl, Tie2 (h) (5-10 mU) is incubated with 8 mM MOPS pH 7.0, 0.2 mM EDTA, 0.5 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

TrkA (h)

In a final reaction volume of 25 μl, TrkA (h) (5-10 mU) is incubated with 8 mM MOPS pH 7.0, 0.2 mM EDTA, 250 μM KKKSPGEYVNIEFG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

TrkB (h)

In a final reaction volume of 25 μl, TrkB (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

TSSK2 (h)

In a final reaction volume of 25 μl, TSSK2 (h) (5-10 mU) is incubated with 8 mM MOPS pH 7.0, 0.2 mM EDTA, 100 μM KKKVSRSGLYRSPSMPENLNRPR, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Yes (h)

In a final reaction volume of 25 μl, Yes (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

ZAP-70 (h)

In a final reaction volume of 25 μl, ZAP-70 (h) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

ZIPK (h)

In a final reaction volume of 25 μl, ZIPK (h) (5-10 mU) is incubated with 8 mM MOPS pH 7.0, 0.2 mM EDTA, 250 μM KKLNRTLSFAEPG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### Example 6

### Effects of Compounds on Ribosomal RNA Synthesis

Assays can also be conducted to determine the effects of compounds on rRNA synthesis from 45S rDNA. Synthesized rRNA is quantified by a polymerase chain reaction (PCR) assay. A primer/probe set can be designed using Primer Express software and synthesized by a commercial supplier, such as Applied Biosystems. A 5′ ETS Probe having the following sequence (at its 3′ end): 6FAM-TTG ATC CTG CCA GTA GC-MGBNFQ is used. Representative primer sequences are as follows:

Forward Primer: CCG CGC TCT ACC TTA CCT ACC T
Reverse Primer: GCA TGG CTT AAT CTT TGA GAC AAG.

A control assay that detects effects of the compounds on C-myc transcription can also be conducted using a primer/probe set, that can be purchased from ABI (TaqMan Gene Expression Assay with assay ID: Hs99999003_m1). The following assay protocol is utilized:

Step 1. Reverse transcription of RNA to DNA

Mix the following:

1 ug RNA

2.5 ul 10× Taq Man buffer

5.5 ul 25 mM MgCl2

5 ul of a mix of dNTP (500 uM each)

1.2 ul random hexamer primer (2.5 uM stock)

0.5 ul RNase inhibitor (0.4 units/ul)

0.6 ul Reverse Transcriptase (1.2 units/ul)

and bring to 25 ul total volume with water. Incubate at 48 degrees C. for 30 minutes. Inactivate Reverse Transcriptase by incubating at 95 for 5 minutes

Step 2. PCR

Mix the following:
5 ul Reverse Transcriptase reaction product
12.5 ul 2×PCR mix
1 uM forward primer
1 uM reverse primer
0.5 uM Taq Man probe
500 nM Rox
Adjust to 25 ul final volume with water

PCR cycles

95 degrees C. 1 minute

40 cycles of

95 degrees C. 15 seconds

60 degrees C. 1 minute.

In addition to assessing c-Myc levels as a control, levels of other gene products can be detected, such as GAPDH.

### Example 7

### Effects of Compounds on Cell Viability

A representative cell-proliferation assay protocol using Alamar Blue dye (stored at 4° C., use 20 ul per well) is described below. This assay monitors the reducing potential of metabolically active proliferating cells: proliferating cells reduce the Alamar Blue to form a fluorescent product, while non-proliferating cells and dying cells do not. Thus the proliferating cells are counted using a fluorescence visualization method to compare the effects of the test compounds. The first procedure hereafter describes a representative assay with HCT-116 cells, and other cell lines can be utilized. For example, a useful colon cancer cell line is colo320, which is a colon adenocarcinoma cell line deposited with the National Institutes of Health as accession number JCRB0225. Parameters for using such cells are available at the http address cellbank.nihs.go.jp/cell/data/jcrb0225.htm. Human cervical cells also may be utilized as described hereafter.

Cell Viability Assay 1

a. Split and trypsinize HCT-116 cells.

b. Count cells using hemocytometer.

c. Plate 4,000-5,000 cells per well in 100 μl of medium and seed into a 96-well plate according to the following plate layout. Add cell culture medium only to wells B10 to B12. Wells B1 to B9 have cells but no compound added.

| | | | |
|---|---|---|---|
| 1 2 3 | 4 5 6 | 7 8 9 | 10 11 12 |

| | |
|---|---|
| A | EMPTY |

| B | NO COMPOUND | | | Medium | |
|---|---|---|---|---|---|
| | ADDED | | | Only | |
| C | 10 nM | 100 nM | 1 uM | 10 uM | QQ58S |
| D | 10 nM | 100 nM | 1 uM | 10 uM | Comp1 |
| E | 10 nM | 100 nM | 1 uM | 10 uM | Comp2 |
| F | 10 nM | 100 nM | 1 uM | 10 uM | Comp3 |
| G | 10 nM | 100 nM | 1 uM | 10 uM | Comp4 |

| | |
|---|---|
| H | EMPTY |

d. Add 100 μl of 2× drug dilution to each well in a concentration shown in the plate layout above. At the same time, add 100 μl of media into the control wells (wells B10 to B12). Total volume is 200 μl/well.

e. Incubate four (4) days at 37° C., 5% CO₂ in a humidified incubator.

f. Add 20 μl Alamar Blue reagent to each well.

g. Incubate for four (4) hours at 37° C., 5% CO₂ in a humidified incubator.

h. Record fluorescence at an excitation wavelength of 544 nm and emission wavelength of 590 nm using a microplate reader.

Cell Viability Assay 2

Human cervical epithelial cells (HeLa cells) are obtained from American Type Culture Collection (Manassas, Va.). Cells are grown in Eagle's minimum essential medium (MEM, Hyclone, Utah) supplemented with 2 mM Glutamine, 0.1 mM nonessential amino acid, 1 mM Na Pyruvate, 1.5 g/L NaHCO₃, 50 mg/L gentamicin, and 10% fetal bovine serum (Hyclone, USA) in a humidified atmosphere of 5% CO₂ at 37° C. Antiproliferative effects of anticancer drugs are tested by the CellTiter 96 AQᵤₑₒᵤₛ assay (Promega, WI), which is a colorimetric assay for determining the number of viable cells. (See, e.g., Wang, L., et al., Methods Cell Sci (1996) 18:249-255). Generally, cells (2,000 to 5,000 cells/well) are seeded on 96 well flat bottom plates (Corning, N.Y.) in 100 μl of culture medium without any anticancer drug on day 0, and the culture medium is exchanged for that contained anticancer drugs at various concentrations on day 1. After incubation for 3 days under normal growth conditions (on day 4), the monolayers are washed once in PBS, and the medium is switched to 100 μl of PBS in each of the 96 well plate. After mixing MTS and PMS at the ratio of 20:1, 20 μl of MTS/PMS solution is added to each of the 96 well plate and incubated for 4 hours in a humidified atmosphere of 5% CO₂ at 37° C. The absorbance is read at 490 nm using FLUOstar Galaxy 96 well plate reader (BMG Labtechnologies, Germany).

Cell Viability Assay 3

A representative assay for detecting cell apoptosis, which makes use of an Annexin V-Alexa 488 staining protocol is performed as follows. Seed 1.5-2.0×106 HCT-116 cells/10 cm dish/10 ml medium. Incubate overnight or up to 24 hrs at 37° C. in a CO₂ incubator. The following day, treat cells with varying concentrations of test compound (e.g., 1, 2, 3, 4 and 5 μM test compound). Maintain one or two untreated plates (medium only) as control plates. The following controls are used: untreated samples (no Alexa or propidium iodide), controls treated with propidium iodide or Alexa 488 only, and controls treated with both Alexa 488 and propidium iodide. Harvest cells (collect attached as well as floating cells). Wash cells twice with cold PBS. Re-suspend cells in 1× Annexin binding buffer. Count cells and dilute in 1× Annexin binding buffer to about 10⁶ cells/0.1 ml, preparing a sufficient volume to have 100 μl per assay. Add 5 μl of the Annexin V conjugate to each 100 μl of cell suspension. Add 4 μl of propidium iodide solution (stock=1 mg/ml) to each 100 μl of cell suspension and incubate the sample at RT for 15 minutes. Add 400 μl Annexin binding buffer, mix gently and keep samples on ice. Analyze stained cells immediately by flow cytometry.

### Example 8

### In Vitro Quadruplex Interaction Characterization

Various methods may be used for in vitro characterization of the compounds of the present invention, including but not limited to i) stop assays; ii) quadruplex/duplex competition assay; iii) quadrome footprints; and iv) direct assay in the absence of a competitor molecule.

Stop Assays. Stop assays are high throughput, first-pass screens for detecting drugs that bind to and stabilize the target G-quadruplex. Generally, DNA template oligonucleotide is created, which contains the nucleotide sequence of the “target” quadruplex against which drug screening is desired. A fluorescently labeled primer DNA is then annealed to the 3′ end of the template DNA. A DNA polymerase such as Taq polymerase is then introduced to synthesize a complementary strand of DNA by extending from the fluorescently labeled primer. When the progress of the Taq polymerase is unhindered, it synthesizes a full-length copy of the template. Addition of a test drug that merely binds to duplex DNA but does not bind selectively the quadruplex region results in a decrease in synthesis of full length product and a concomitant increase in variable-length DNA copies. If, however, the test drug selectively binds to and stabilizes the quadruplex, the progress of polymerase arrests only at the quadruplex, and a characteristic “Stop Product” is synthesized.

Compounds are initially screened at a single concentration, and “hits” are re-assayed over a range of doses to determine an IC₅₀ value (i.e., the concentration of drug required to produce an arrest product/full-length product ratio of 1:1). These products are visualized by capillary electrophoresis. In one assay embodiment, a 5′-fluorescent-labeled (FAM) primer (P45, 15 nM) was mixed with template DNA (15 nM) in a Tris-HCL buffer (15 mM Tris, pH 7.5) containing 10 mM MgCl₂, 0.1 mM EDTA and 0.1 mM mixed deoxynucleotide triphosphates (dNTP's). In one example, the FAM-P45 primer (5′-6FAM-AGTCTGACTGACTGTACGTAGCTAATACGACTCACTATAG CAATT-3′) (SEQ ID NO. 17) and the c-Myc template DNA (5′-TCCAACTATGTATACTGGGG AGGGTGGGGAGGGTGGGGAAGGTTAGCGACACGCAATTGCTATAGTGAGTCGTATT AGCTACGTACAGTCAGTCAGACT-3′) (SEQ ID NO. 18) were synthesized and HPLC purified by Applied Biosystems. The mixture was denatured at 95° C. for 5 minutes and, after cooling down to room temperature, was incubated at 37° C. for 15 minutes.

After cooling down to room temperature, 1 mM KCl₂ and the test compound (various concentrations) were added and the mixture incubated for 15 minutes at room temperature. The primer extension was performed by adding 10 mM KCl and Taq DNA Polymerase (2.5 U/reaction, Promega) and incubating at 70° C. for 30 minutes. The reaction was stopped by adding 1 μl of the reaction mixture to 10 μl Hi-Di Formamide mixed and 0.25 μl LIZ120 size standard. Hi-Di Formamide and LIZ120 size standard were purchased from Applied Biosystems. The partially extended quadruplex arrest product was between 61 or 62 bases long and the full-length extended product was 99 bases long. The products were separated and analyzed using capillary electrophoresis. Capillary electrophoresis was performed using an ABI PRISM 3100-Avant Genetic Analyzer. The assay was performed using compounds described above and results are shown in Table 1. μM concentrations reported in Table 1 are concentrations at which 50% of the DNA was arrested in the assay (i.e., the ratio of shorter partially extended DNA (arrested DNA) to full-length extended DNA is 1:1).

Quadruplex/Duplex Competitor Assay. The selectivity of compounds for the target quadruplex sequence relative to duplex DNA may be measured using a competition assay (i.e., “selectivity screen”). This selectivity screen uses the stop assay as a reporter system to measure the relative ability of an externally added DNA sequence to compete with the target quadruplex structure formed in the DNA template for binding of the drug. For example, the competitors are the c-myc quadruplex sequence, which is identical to the quadruplex sequence present in the template DNA; or a plasmid DNA which mimics complex genomic duplex DNA. The degree to which each competitor successfully “soaks up” drug in solution is reflected by the quantitative decrease in synthesis of the stop product. In this manner, the relative binding affinities of drug to both the target quadruplex and duplex DNA are determined. In certain assays, the G-quadruplex binding ligand is added at the concentration previously established to produce a 1:1 ratio of stop-product to full-length product. A CC50 for each nucleic acid competitor is defined as the concentration of competitor required to change the ratio of arrest product to full-length product from 1:1 to 1:2. Representative nucleic acid sequences for use in this assay are set forth hereafter in Table 4.

TABLE 4
TGFB3-81
TATACGGGGTGGGGGAGGGAGGGATTAGCGACACGCAATTGCTATAGTGA
GTCGTATTAGCTACGTACAGTCAGTCAGACT
HRAS-85
TATACCGGGGCGGGGCGGGGGCGGGGGCTTAGCGACACGCAATTGCTAT
AGTGAGTCGTATTAGCTACGTACAGTCAGTCAGACT
BCL2-97(full)
TAGGGGCGGGCGCGGGAGGAAGGGGGCGGGAGCGGGGCTGTTAGCGACA
CGCAATTGCTATAGTGAGTCGTATTAGCTACGTACAGTCAGTCAGACT
HMGA-97
TTAGAGAAGAGGGGAGGAGGAGGAGGAGAGGAGGAGGCGCTTAGCGACAC
GCAATTGCTATAGTGAGTCGTATTAGCTACGTACAGTCAGTCAGACT
MYC99
TCCAACTATGTATACTGGGGAGGGTGGGGAGGGTGGGGAAGGTTAGCGA
CACGCAATTGCTATAGTGAGTCGTATTAGCTACGTACAGTCAGTCAGACT
IMOTIF99
TCCAACTATGTATACCCTTCCCCACCCTCCCCACCCTCCCCATTAGCGAC
ACGCAATTGCTATAGTGAGTCGTATTAGCTACGTACAGTCAGTCAGACT
Humtel-95
TCATATATGACTACTTAGGGTTAGGGTTAGGGTTAGGGTTACTGCCACGC
AATTGCTATAGTGAGTCGTATTAGCTACGTACAGTCAGTCAGACT
SRC89
ATGATCACCGGGAGGAGGAGGAAGGAGGAAGCGCGCTGCCACGCAATT
GCTATAGTGAGTCGTATTAGCTACGTACAGTCAGTCAGACT
Primer (45 MER):
AGTCTGACTGACTGTACGTAGCTAATACGACTCACTATAG
CAATT

Quadrome Footprints. Compounds may also be evaluated for their ability to bind to other native quadruplex structures of biological relevance, including quadruplex control elements that regulate a range of different oncogenes. The resulting data are used to create a Quadrome footprint.

Direct Interaction Assay. Compounds may be evaluated for their ability to interact directly with nucleic acids capable of forming a quadruplex structure, wherein the nucleic acid is not a telomeric nucleic acid. The assay may be performed in the same or different vessels. For example, a compound may be contacted with each nucleic acid in the same vessel. Alternatively, a compound may be separately contacted with each of the nucleic acids tested in a different vessel. A telomeric nucleic acid as used herein represents a region of highly repetitive nucleic acid at the end of a chromosome. As used herein, a direct interaction is measured without the presence of a competitor nucleic acid.

An interaction between the compound and the nucleic acid may be determined for example, by measuring IC₅₀ values, which are indicative of the binding and/or quadruplex stabilization. The selectivity of interactions may be determined, for example, by comparing measured IC₅₀ values. For example, the lowest IC₅₀ values may be used to indicate a strong interaction between the compound and the nucleic acid, while highest IC₅₀ values show a poor interaction; thus, showing selectivity of interaction. The reaction products may be characterized by capillary electrophoresis.

Transcription Reporter Assay. In a transcription reporter assay, test quadruplex DNA is coupled to a reporter system, such that a formation or stabilization of a quadruplex structure can modulate a reporter signal. An example of such a system is a reporter expression system in which a polypeptide, such as luciferase or green fluorescent protein (GFP), is expressed by a gene operably linked to the potential quadruplex forming nucleic acid and expression of the polypeptide can be detected. As used herein, the term “operably linked” refers to a nucleotide sequence which is regulated by a sequence comprising the potential quadruplex forming nucleic acid. A sequence may be operably linked when it is on the same nucleic acid as the quadruplex DNA, or on a different nucleic acid. An exemplary luciferase reporter system is described herein.

A luciferase promoter assay described in He, et al., Science (1998) 281:1509-1512 often is utilized for the study of quadruplex formation. Specifically, a vector utilized for the assay is set forth in reference 11 of the He, et al., document. In this assay, HeLa cells are transfected using the lipofectamin 2000-based system (Invitrogen) according to the manufacturer's protocol, using 0.1 μg of pRL-TK (Renilla luciferase reporter plasmid) and 0.9 μg of the quadruplex-forming plasmid. Firefly and Renilla luciferase activities are assayed using the Dual Luciferase Reporter Assay System (Promega) in a 96-well plate format according to the manufacturer's protocol.

Circular Dichroism Assay. Circular dichroism (CD) is utilized to determine whether another molecule interacts with a quadruplex nucleic acid. CD is particularly useful for determining whether a PNA or PNA-peptide conjugate hybridizes with a quadruplex nucleic acid in vitro. PNA probes are added to quadruplex DNA (5 μM each) in a buffer containing 10 mM potassium phosphate (pH 7.2) and 10 or 250 mM KCl at 37° C. and then allowed to stand for 5 minutes at the same temperature before recording spectra. CD spectra are recorded on a Jasco J-715 spectropolarimeter equipped with a thermoelectrically controlled single cell holder. CD intensity normally is detected between 220 nm and 320 nm and comparative spectra for quadruplex DNA alone, PNA alone, and quadruplex DNA with PNA are generated to determine the presence or absence of an interaction (see, e.g., Datta, et al., JACS (2001) 123:9612-9619). Spectra are arranged to represent the average of eight scans recorded at 100 nm/min

Fluorescence Binding Assay. An example of a fluorescence binding assay is a system that includes a quadruplex nucleic acid, a signal molecule, and a test molecule. The signal molecule generates a fluorescent signal when bound to the quadruplex nucleic acid (e.g., N-methylmesoporphyrin IX (NMM)), and the signal is altered when a test compound competes with the signal molecule for binding to the quadruplex nucleic acid. An alteration in the signal when test molecule is present as compared to when test compound is not present identifies the test compound as a quadruplex interacting compound.

50 μl of quadruplex nucleic acid or a nucleic acid not capable of forming a quadruplex is added in 96-well plate. A test compound also is added in varying concentrations. A typical assay is carried out in 100 μl of 20 mM HEPES buffer, pH 7.0, 140 mM NaCl, and 100 mM KCl. 50 μl of the signal molecule NMM then is added for a final concentration of 3 μM. NMM is obtained from Frontier Scientific Inc, Logan, Utah. Fluorescence is measured at an excitation wavelength of 420 nm and an emission wavelength of 660 nm using a FluoroStar 2000 fluorometer (BMG Labtechnologies, Durham, N.C.). Fluorescence often is plotted as a function of concentration of the test compound or quadruplex-targeted nucleic acid and maximum fluorescent signals for NMM are assessed in the absence of these molecules.

Gel Electrophoretic Mobility Shift Assay (EMSA). An EMSA is useful for determining whether a nucleic acid forms a quadruplex and whether a nucleotide sequence is quadruplex-destabilizing. EMSA is conducted as described previously (Jin & Pike, Mol. Endocrinol. 10: 196-205 (1996)) with minor modifications. Generally, synthetic single-stranded oligonucleotides are labeled in the 5′-terminus with T4-kinase in the presence of [γ-³²P] ATP (1,000 mCi/mmol, Amersham Life Science) and purified through a sephadex column ³²P-labeled oligonucleotides (30,000 cpm) are then incubated with or without various concentrations of a testing compound in 20 μl of a buffer containing 10 mM Tris pH 7.5, 100 mM KCl, 5 mM dithiothreitol, 0.1 mM EDTA, 5 mM MgCl₂, 10% glycerol, 0.05% Nonedit P-40, and 0.1 mg/ml of poly(dI-dC) (Pharmacia). After incubation for 20 minutes at room temperature, binding reactions are loaded on a 5% polyacrylamide gel in 0.25× Tris borate-EDTA buffer (0.25×TBE, 1×TBE is 89 mM Tris-borate, pH 8.0, 1 mM EDTA). The gel is dried and each band is quantified using a phosphoimager.

DMS Methylation Protection Assay. Chemical footprinting assays are useful for assessing quadruplex structure. Quadruplex structure is assessed by determining which nucleotides in a nucleic acid are protected or unprotected from chemical modification as a result of being inaccessible or accessible, respectively, to the modifying reagent. A DMS methylation assay is an example of a chemical footprinting assay. In such an assay, bands from EMSA are isolated and subjected to DMS-induced strand cleavage. Each band of interest is excised from an electrophoretic mobility shift gel and soaked in 100 mM KCl solution (300 μl) for 6 hours at 4° C. The solutions are filtered (microcentrifuge) and 30,000 cpm (per reaction) of DNA solution is diluted further with 100 mM KCl in 0.1×TE to a total volume of 70 μl (per reaction). Following the addition of 1 μl salmon sperm DNA (0.1 μg/μl), the reaction mixture is incubated with 1 μl DMS solution (DMS:ethanol; 4:1; v:v) for a period of time. Each reaction is quenched with 18 μl of stop buffer (b-mercaptoethanol:water:NaOAc (3 M); 1:6:7; v:v:v). Following ethanol precipitation (twice) and piperidine cleavage, the reactions are separated on a preparative gel (16%) and visualized on a phosphoimager.

### Example 9

### Cytochrome P450 (CYP450) Inhibition Assay

The compounds of the present invention may be evaluated for potential inhibitory activity against cytochrome P450 isoenzymes. Generally, six reaction tubes with 100 μL of a solution containing 50 mM potassium phosphate, pH 7.4, 2.6 mM NADP+, 6.6 mM glucose 6-phosphate, 0.8 U of glucose 6-phosphate dehydrogenase/mL and 1:6 serial dilutions of the test compound will be prepared along with six tubes of 1:6 serial dilutions of a suitable positive control inhibitor. The reactions will be initiated by adding 100 μL of a pre-warmed enzyme/substrate solution to the reaction tubes. A zero time-point control reaction will be prepared by adding 50 μL of acetonitrile to 100 μL of cofactor solution to inactivate the enzymes, then adding 100 μL of enzyme/substrate solution. A control reaction with no inhibitor may also be prepared. After a suitable incubation at 37 C, the reactions will be terminated by the addition of 50 μL of acetonitrile. The reactions will be analyzed for the metabolite forms of the probe substrate using LC/MS/MS.

### Example 10

### Evaluation of Compound Efficacy in Tumor Suppression

A representative study for evaluating the efficacy of compounds of the present invention in athymic nude mouse models of human carcinoma is as follows. Male or female animals (mouse, Taconic) (NCR, nu/nu) aged five to six weeks and weighing more than 20 grams will be used. The animals are purposely bred and will be experimentally naïve at the outset of the study. Tumors are propagated either from injected cells or from the passage of tumor fragments. Cell lines that can be utilized include, but are not limited to, HCT116, alia Paca-2, HPAC, Hs700T, Panc10.05, Panc 02.13, PL45, SW 190, Hs 766T, CFPAC-1 and PANC-1.

Cell implantation. One to ten million cells suspended in 0.1 ml culture media with or without Matrigel (Collaborative Biomedical Products, Inc, Bedford, Mass.) are inoculated subcutaneously in the right flank of animals. There generally is one injection per animal. Within 7-14 days of injection tumors develop to a study use size of approximately 1.0 cm³. Donors and tumors often are grown 10-28 days and to a size of 1.5 cm³ in order to be used for serial transplantation.

Fragment transplantation. Donor animals are euthanized and tumors surgically excised and cut into 2 mm³ size fragments using aseptic technique Animals to be implanted are lightly anesthetized with isoflurane. The area implanted is cleansed with 70% alcohol and betadine. A single fragment is implanted subcutaneously using a trocar.

Efficacy studies. Tumor bearing animals are randomly divided. For example, in a representative study, animals may be randomly divided into groups containing 5-10 animals each, as described in Table 5.

TABLE 5

| | | | | Dose | Number |
|---|---|---|---|---|---|
| | Number | | | Solution | Euthanized |
| Group | of Males/ | | Dose Vol. | Conc. | on: |
| No. | Females | Dose Level | (μL) | (mg/mL) | Day 28-42 |
| 1 | N = 5-10 | Negative Control* | 250 | | all |
| 2 | N = 5-10 | Positive Control** | 10-400 IP | 2 to 5 IP | all |
| | | | 10-250 IV | 2.5 to 5 IV | |
| | | | 125-500 PO | ≦10 PO | |
| | | Test Compound | | | |
| Groups 3-8 | N = 5-10/ | 1 to 25 IP | 10-400 IP | 2.5 to 5 IP | all |
| | grp | 1 to 50 IV | 10-250 IV | 2.5 to 5 IV | |
| | <56 total | 50 to 200 PO | 125-500 PO | 10 PO | |

*Vehicle/Diluent
**Commercially available anticancer compounds including, but not limited to, Taxol, CPT11 and Gemcitabine will be used as positive controls.

Dosing Procedure. Compounds will be administered QD, QOD, Q3D or once weekly via IP, IV (lateral tail vein) or PO. Animals will be dosed in a systematic order that distributes the time of dosing similarly across all groups. For bolus IP and PO dosing, animals will be manually restrained. For IV bolus dosing or short term IV infusion (one minute), animals will be mechanically restrained but not sedated. Disposable sterile syringes will be used for each animal/dose.

Efficacy studies for two exemplary compounds of the invention in an HCT-116 xenograft model are shown in FIGS. 3A and 3B. Both results show that exemplary compounds of the invention exhibit anti-tumor activity in an HCT-116 xenograft model.

### Example 11

### Evaluation of Maximum Tolerated Doses

A representative experiment for evaluating the maximum tolerate dose (MTD) of compounds of the present invention may be designed as follows. Selection for animal models is as described herein.

Acute Toxicity Studies. In a representative study to determine the MTD after a single dose, sixty naive animals, for example, will be randomly divided into groups containing 10 animals (5 male and 5 female) and will receive either one compound via two routes of administration or two compounds via a single route of administration. A single 50 mg/kg IV dose has been shown to be tolerated, and is used as the preliminary low dose levels. The low dose for oral studies is based on projected tolerability and will be adjusted downward if necessary. A representative design of dose levels, dose volumes and dose solution concentration are described in Table 6.

TABLE 6

| | Number | | | Dose | |
|---|---|---|---|---|---|
| | of Males | | | Solution | Number |
| | and | Dose Level | Dose Vol. | Conc. | Euthanized on: |
| Group No. | Females | (mg/kg) | (μL) | (mg/mL) | Day 7 |
| | | Test compound #1 | | | |
| 1 | N = 5 M | 50 IV | 250 IV | 5 IV | all |
| | N = 5 F | 100 PO | 500 PO | 5 PO | all |
| 2 | N = 5 M | 75 IV | 250 IV | 8.25 IV | all |
| | N = 5 F | 200 PO | 500 PO | 10 PO | |
| 3 | N = 5 M | 100 IV | 250 IV | 10 IV | all |
| | N = 5 F | 300 PO | 500 PO | 15 PO | |
| | | Test compound #2 | | | |
| 4 | N = 5 M | 50 IV | 250 IV | 5 IV | all |
| | N = 5 F | 100 PO | 500 PO | 5 PO | |
| 5 | N = 5 M | 75 IV | 250 IV | 8.25 IV | all |
| | N = 5 F | 200 PO | 500 PO | 10 PO | |
| 6 | N = 5 M | 100 IV | 250 IV | 10 IV | all |
| | N = 5 F | 300 PO | 500 PO | 15 PO | |

SubChronic Studies. In a representative study to characterize dose-response relationships following repeated dosing, twenty-five naive animals, for example, will be randomly divided into groups containing 5 animals each as described in Table 7. Each two week study will test only one compound via a single route of administration at an optimal dose derived from data collected in prior acute toxicity studies.

TABLE 7

| | Number | | | Dose | |
|---|---|---|---|---|---|
| | of Males | | | Solution | Number |
| | and | Dose Level | Dose Vol. | Conc. | Euthanized on: |
| Group No. | Females | (mg/kg) | (μL) | (mg/mL) | Day 14 |
| 1 | N = 5 | Negative Control | 250 IV | Depends on | all |
| | | | 500 PO | Dose Level | |
| 2 | N = 5 | Test Compound | 250 IV | Depends on | all |
| QD | | As Determined in | 500 PO | Dose Level | |
| | | MTD Studies | | | |
| 3 | N = 5 | Test Compound | 250 IV | Depends on | all |
| QOD | | As Determined in | 500 PO | Dose Level | |
| | | MTD Studies | | | |
| 4 | N = 5 | Test Compound | 250 IV | Depends on | all |
| Q3D | | As Determined in | 500 PO | Dose Level | |
| | | MTD Studies | | | |
| 5 | N = 5 | Test Compound | 250 IV | Depends on | all |
| Q7D | | As Determined in | 500 PO | Dose Level | |
| | | MTD Studies | | | |

Dosing Procedure. Compounds will be administered QD, QOD, Q3D or Q7D via IV (lateral tail vein) or PO. Animals will be dosed in a systematic order that distributes the time of dosing similarly across all groups. For PO dosing, animals will be manually restrained. For IV bolus dosing or short term IV infusion (one minute), animals will be mechanically restrained but not sedated. Disposable sterile syringes will be used for each animal/dose.

### Example 12

### Evaluation of Pharmacokinetic Properties

A representative pharmacokinetic study for evaluating pharmacokinetic properties of the compounds herein may be designed as follows. Male animals (mouse, Balb/c or rat, SD) aged five to six weeks. For rat models, rats weighing more than 200 grams will be used. In a representative study, twenty animals, for example, will randomly divided into 4 groups, as shown in Table 8. One group with be untreated and samples taken to be used as a base line. The other three groups will be and administered a single dose of compounds by intravenous injection.

TABLE 8

| Group | No. of | Time followed by injection |
|---|---|---|
| No. | Animals | (h) |
| 1 | 2 | Naïve |
| 2 | 6 | .25, 2, 8 |
| 3 | 6 | .5, 4, 12 |
| 4 | 6 | 1, 6, 24 |

Dosing Procedure. Compounds will be administered via IV (lateral tail vein), IP or PO. Animals will be dosed in a systematic order that distributes the time of dosing similarly across all groups. For IP and PO dosing, animals will be manually restrained. For IV bolus dosing or short term IV infusion (one minute), animals will be mechanically restrained but not sedated. Disposable sterile syringes will be used for each animal/dose.

Approximately 0.5 ml of blood will be collected from the naive animals via cardiac puncture prior to the first dose Terminal blood samples (0.5 ml) will be collected via cardiac puncture from two animals per group per time point according to the above chart. All samples will be placed in tubes containing lithium heparin as anticoagulant and mixed immediately by inverting. They will be centrifuged and the plasma flash frozen in liquid nitrogen, stored at −70° C. or greater and analyzed for drug levels.

### Example 13

### Determination of In Vitro Metabolic Stability in Hepatocytes

A representative protocol to determine the stability of a new chemical entity in the presence of hepatocytes (human, rat, dog, monkey) in in vitro incubations may be designed as follows. The test article will be incubated with hepatocytes and suitable media for various times at 37° C. The reaction mixtures will be extracted and analyzed by LC/MS/MS for the parent compound and anticipated metabolites. If applicable, a half-life will be calculated for the consumption of the test article. Metabolism controls will be run for comparison of the half-life values with that obtained for the test article. The metabolism controls may be tolbutamide, desipramine and naloxone, which have defined pharmacokinetics corresponding to low, moderate and high in vivo clearance values, respectively.

Metabolic Stability Study. Generally, solutions of the test compounds will be prepared along with a cocktail solution of metabolism controls that are intended to provide a reference for enzyme activity. The reactions will be initiated by combining these pre-warmed solutions with hepatocyte suspensions and with a media control solution. Control zero samples will be taken from these reactions immediately after initiation. Additional samples may be taken at appropriate time points. Each sample will be immediately placed in a terminating solution (acidified MeCN containing IS) to stop the reaction. Hepatocyte blank suspensions and test compound standard solutions will be prepared.

Samples and standards for the test compound as well as appropriate blanks may be subjected to a custom sample preparation procedure and analyzed for the parent and/or metabolite form of the test compound using HPLC coupled with tandem mass spectrometry. Samples and standards for the metabolism controls may be subjected to the analytical method described herein. Where Krebs Henseleit buffer will be added, the buffer is bubbled with 5% CO₂ in air at room temperature for 5-10 minutes before adding BSA to a final concentration of 0.2% w/v. The volume of terminating solution and the method of sample preparation will be determined for the test article during method development.

Test Article/Media Solution. A solution of the test article will be prepared by adding an appropriate volume of the stock solution to 0.2% BSA in Krebs Henseleit buffer equilibrated with 5% CO₂ in air. The final concentration will be between 5 μM and 20 μM, and the final assay concentration at initiation of the reactions will be between 1 μM and 10 μM.

Metabolism Controls/Media Solution. A solution of tolbutamide, desipramine and naloxone will be prepared by adding an appropriate volume of each 10 mM stock solution to 0.2% BSA in Krebs Henseleit buffer equilibrated with 5% CO₂ in air. The final concentration will be 20 μM for each metabolism control and the final assay concentration will be 10 μM at initiation of the reactions.

Hepatocyte Suspension Solution. The hepatocytes will be thawed and isolated according to the vendor (Invitrotech, Inc.) instructions. During the final step of the procedure, the viability of the cells will be determined using the method of trypan blue exclusion. Then, the hepatocytes will be resuspended with 0.2% BSA in Krebs Henseleit buffer equilibrated with 5% CO₂ in air so the final concentration is 0.5 million viable cells/mL. The concentration at the initiation of the reactions will be 0.25 million viable cells/mL.

Initiating Test Article Incubation. Equal volumes of the test article solution prepared in step 2.1.3 will be dispensed into four polypropylene scintillation vials. The vials are pre-warmed for 5-10 minutes at 37° C. with 95% humidity and 5% CO₂. Equal volumes of 0.2% BSA in Krebs Henseleit buffer equilibrated with 5% CO₂ in air will be added to two of the vials and mixed thoroughly. Immediately after initiating the reaction, a timer is started and a 100 μL sample is removed from each vial and placed into a 1.7-mL centrifuge tube containing a suitable volume of terminating solution. These samples will serve as media controls to check for non-enzymatic degradation and non-specific binding to the vessel.

Equal volumes of the hepatocyte suspension prepared above will be added to two of the vials and mixed thoroughly. Immediately after initiating the reaction, a timer is started and a 100 μL sample is removed from each vial and placed into a 1.7-mL centrifuge tube containing a suitable volume of terminating solution. All vials are placed in an incubator maintained at 37° C., 95% humidity and 5% CO₂.

Initiating Metabolism Control Incubation. Equal volumes of the metabolism control solution prepared above will be dispensed into two polypropylene scintillation vials. The vials are pre-warmed for 5-10 minutes at 37° C. with 95% humidity and 5% CO₂. Equal volumes of the hepatocyte suspension prepared above will be added to each of the two vials and mixed thoroughly. Immediately after initiating the reaction, a timer is started and a 100 μL sample is removed from each vial and placed into a 1.7-mL centrifuge tube containing an equal volume of terminating solution. All vials are placed in an incubator maintained at 37° C., 95% humidity and 5% CO₂.

Sample Collection. The vials will be gently shaken and samples (100 μL) will be removed and placed into a 1.7-mL centrifuge tube containing an appropriate volume of terminating solution according to the following schedule: Test article samples are taken after 5, 10, 15, 30, 60, 90 and 120 minutes; metabolism control samples are taken after 30, 60, 90 and 120 minutes. Immediately after removal of the samples, the vials are placed back in the incubator until the last sample is collected.

Blank Preparation. A sample (100 μL) of the hepatocyte suspension will be added to an equal volume of 0.2% BSA in Krebs Henseleit buffer and mixed thoroughly. A 100 μL sample of this solution will be removed and placed into a 1.7-mL centrifuge tube containing the same volume of terminating solution used for the test article reaction. A sample of the incubation medium (0.2% BSA in Krebs Henseleit buffer) will be placed into a 1.7-mL centrifuge tube containing the same volume of terminating solution used for the test article reaction.

Sample Preparation and Analysis. All vials will be centrifuged at 16,000 g for 3 minutes. The supernatants will be placed into polypropylene autosampler vials and stored at 4° C. (<1 day) or −70° C. (>1 day) until analysis. The test article solutions will be analyzed using HPLC/MS/MS conditions according to standard procedures. In one example, the following HPLC conditions may be used: column (Phenomenex Synergi Hydro-RP, 100.0×2.0 mm, 5 μm); guard column (Phenomenex C18, 4.0×2.0 mm, 5 μm); flow rate (0.3 mL/min); column temperature at 45° C.; injection volume at 10 μL; and ambient autosampler temperature.

### Example 14

### Determination of In Vitro Metabolic Stability in Microsomes

A representative protocol to determine the stability of a new chemical entity in the presence of liver microsomes (human, rat, dog, monkey) in in vitro incubations may be designed as follows. The test article will be incubated with microsomes and suitable media for various times at 37° C. The reaction mixtures will be extracted and analyzed by LC/MS/MS for the parent compound and anticipated metabolites. If applicable, a half-life will be calculated for the consumption of the test article. Metabolism controls will be run for comparison of the half-life values with that obtained for the test article. The metabolism controls are tolbutamide, desipramine and testosterone, and these compounds have defined pharmacokinetics corresponding to low, moderate and high in vivo clearance values, respectively.

Metabolic Stability Study. Generally, six pre-warmed reaction vials with 100 μL of a solution containing 50 mM potassium phosphate, pH 7.4, 2.6 mM NADP⁺, 6.6 mM glucose 6-phosphate, 0.8 U/mL of glucose 6-phosphate dehydrogenase and 1, 10 or 50 μM of the test compound are prepared. Similar reactions with metabolic controls representing low (tolbutamide), moderate (desipramine), and high (testosterone) clearance compounds are run simultaneously with the same enzyme solution. The reactions are initiated by adding 100 μL of a pre-warmed enzyme solution and incubated at 37° C. The zero time-point reaction is prepared by adding 50 μL of acetonitrile (containing internal standard) to the test compound/cofactor solution prior to adding the enzyme solution. After 15, 30, 60, 90 and 120 minutes, a reaction tube is removed from the water bath and the reaction is terminated with 50 μL of acetonitrile containing internal standard. The reactions are extracted and the samples are analyzed for the parent form of the test compound and one metabolite using a C18 column with MS/MS detection. Each assay is performed in duplicate.

Cofactor/Test compound Solution Concentrations. A stock solution of 10 mM NCE will be prepared in 10% DMSO (v/v). For all assays, a 2, 20 or 100 μM solution of the test article will be prepared in 50 mM potassium phosphate, pH 7.4, 2.6 mM NADP⁺, 6.6 mM glucose 6-phosphate and 0.8 U/mL of glucose 6-phosphate dehydrogenase (cofactor solution).

Cofactor/Metabolism Control Solution Concentrations. Stock solutions of the metabolism controls (tolbutamide, desipramine, and testosterone) will be used to prepare a 6 μM solution of the metabolism control in cofactor solution described in step

Enzyme Solution Concentrations. The enzyme solutions will be prepared by adding liver microsomes to 50 mM potassium phosphate, pH 7.4, to a final concentration of 1 mg/mL. All microsomes were purchased from XenoTech or InvitroTech, Inc.

Initiating the Reactions. All the reaction tubes will be pre-warmed at 37° C. in a water bath for about 3-5 minutes. The zero time-point control reaction will be prepared for each replicate by adding 50 μL of acetonitrile containing 15.9 μM nebularine (internal standard) to 100 μL of cofactor solution to inactivate the enzymes, and then vortex mixing. The reactions will be initiated by adding 100 μL of the enzyme solution to each of the tubes and vortex mixing. All the tubes, including the zero time-point control, will be incubated in a 37° C. water bath. The final concentrations of all components in the tubes after initiating the reactions are 50 mM potassium phosphate, pH 7.4, 1.3 mM NADP⁺, 3.3 mM glucose 6-phosphate, 0.4 U/mL of glucose 6-phosphate dehydrogenase, 0.5 mg/mL liver microsomes and 1, 10 or 50 μM test article.

Terminating and Extracting the Reactions. After 15, 30, 60, 90 and 120 minutes at 37° C., the reactions will be terminated by the addition of 150 μL of acetonitrile containing 15.9 μM nebularine (internal standard). The zero time-point control is removed from the water bath after 120 minutes. All vials will be centrifuged at 16,000 g for 3 minutes. The supernatants will be placed into polypropylene autosampler vials and stored at 4° C. (<1 day) or −70° C. (>1 day) until analysis.

Analysis of Test Article Solutions. The test article solutions will be analyzed using HPLC/MS/MS conditions according to standard procedures.

### Example 15

### Bacterial Mutagenicity Test

This Mutagenicity Assessment assay (Ames Assay) will evaluate the potential of the test article extracts to induce histidine (his) reversion in S. typhimurium (his− to his+) or tryptophan (trp) reversion in E. coli (trp− to trp+) caused by base changes or frameshift mutations in the genome of tester organisms. Generally, a plate incorporation assay will be conducted with five strains of Salmonella typhimurium (TA97a, TA98, TA100, TA102, and TA1535) and one strain of Escherichia coli (WP2-uvrA⁻) in the presence and absence of an exogenous mammalian activation system (S9). The test article will be dissolved in 5% dextrose. A series of dilutions will then be prepared in saline just prior to testing. A Range Finding Study will also be conducted for this assay to determine the appropriate doses for definitive mutagenicity assessment.

Test Material Preparation

A stock solution of test article will be prepared at 20.0 mg/mL as follows: 1.0 g test article will be added to 15.0 mL of 0.1 HCl for 1 minute. The test article will be stirred for 15 minutes at room temperature. Next 33.0 mL of deionized water will be added and allowed to stir for 30 minutes. The pH will then be adjusted to 3.53. Lower doses will be prepared by dilution in 5% dextrose from this stock immediately prior to use. To minimize any change of degradation, the test article solutions will be kept on ice after preparation and until just prior to dosing procedures. The test article will be administered in vitro, through a solvent compatible with the test system.

Genotypic Characterization of the Test Strains

Working stocks of test strains will be confirmed for genotypic markers and acceptable spontaneous reversion rates. All working stocks should demonstrate a requirement for histidine or tryptophan (E. coli only). Additionally, the following conformations will be made with each assay, as appropriate: sensitivity to crystal violet due to the rfa wall mutation; sensitivity to ultraviolet light due to the deletion of the uvrB gene (uvrA in E. coli), resistance to ampicillin due to the presence of the pKM101 plasmid; and resistance to tetracycline due to the presence of the pAQ1 plasmid. Spontaneous reversion rates for the strains will be determined using the negative controls.

Test articles that are water-soluble will be dissolved in isotonic saline or other suitable solvent. Test articles that are not water-soluble will be dissolved in dimethylsulfoxide (DMSO) or other suitable solvent. If DMSO is anticipated to cause adverse reactions with the test article, the test article will be suspended in carboxymethylcellulose. In order to aid in dissolution, heating, vigorous vortexing or alternative solvents may be employed.

Test System

This assay will be conducted in accordance with the plate incorporation methodology originally described by Ames (Ames et al., Mutation Research (1975) 31:347-364) and updated by Maron and Ames (Maron et al., Mutation Research (1983) 113:173-215). This assay has historically been used to detect mutation in a gene of a histidine requiring strain to produce a histidine independent strain or concordantly, to detect mutation in a gene of a tryptophan requiring strain to produce a tryptophan independent strain. In addition, it has been shown to detect diverse classes of chemical mutagens which produce heritable DNA mutations of a type which are associated with adverse effects.

The Salmonella typhimurium strains that may be used in this assay, TA97a, TA98, TA100, and TA102 are described by Maron and Ames, supra; Green et al., Mutation Research (1976) 38:33-42); and Brusick et al., Mutation Research (1980) 76:169-190)). S. typhimurium strain TA1535 and E. coli strain Wp2-uvrA⁻ may be obtained from American Type Culture Collection, Manassas, Va. (ATCC numbers: 29629 and 49979, respectively). All working stocks of test strains will be confirmed for genotypic markers and acceptable reversion rates. Working stocks should demonstrate a requirement for histidine or tryptophan (E. coli only).

Experimental Methods

Master plates of the tester strains will be prepared from frozen working stocks. To create working cultures for each bacterial strain used in the assay, a single colony will be transferred from the master plate into Oxoid nutrient broth and incubated, with shaking, at 37±2° C. until an optical density (at 650 nm) of 0.6-1.6 is reached. This overnight culture will be used for the mutagenicity test and for genotypic confirmation. Genotype tests will be performed as described in the protocol.

For both the dose range and mutagenicity test, a top agar consisting of 0.6% Difco agar in 0.5% NaCl will be melted and a solution of 0.5 mM L-histidine/0.5 mM biotin or 0.5 mM L-tryptophan will be added to the melted top agar at a ratio of 10 mL per 100 mL agar. The supplemented agar will be aliquotted, 2 mL per tube and held at 45-47° C. To prepare the top agar for treatment, 0.1 mL of the test article or control, 0.1 mL of the bacterial culture and 0.5 mL of phosphate buffered saline will be added to the molten agar. The mixture will be briefly vortexed and poured onto a room temperature minimal glucose agar plate (1.5% Difco agar, 2% glucose, in Vogel-Bonner medium E). Metabolic activation will be provided by adding 0.5 mL of the S9 mix in place of the PBS. The plates will be allowed to harden and then incubated 48-72 hours at 37±2° C. All plates will be counted using an automatic image analysis system. Negative control and test article treated plates will also be examined for the presence of a bacterial lawn.

Exogenous Metabolic Activation

The in vitro metabolic activation system used in this assay is comprised of Sprague Dawley rat liver enzymes and a cofactor pool. The enzymes will be contained in a preparation of liver microsomes (S9 fraction) from rates treated with Arochlor to induce the production of enzymes capable of transforming chemicals to more active forms. Immediately prior to use, the S9 will be thawed and mixed with a cofactor pool to contain 5% S9, 5 mM glucose 6-phosphate, 4 mM β-nicotine-adenine dinucleotide phosphate, 8 mM MgCl₂ and 33 mM KCl in a 200 mM phosphate buffer at pH 7.4.

Dose Levels and Replicates

The test article will be tested in triplicate at five dose levels (20.0, 10.0, 5.0, 2.5, and 1.25 mg/mL) along with appropriate vehicle (5% dextrose) and positive controls in the dose range assay. This is equivalent to 2.0, 1.0, 0.5, 0.25, and 0.125 mg/plate.

For the definitive assay, three dose levels will be chosen (10.0, 10.0, and 5.0 mg/mL), which is equivalent to 2.0, 1.0, and 0.5 mg/plate. All treatments, including negative and positive control, will be plated in triplicate against test strains TA97a, TA98, TA100, TA102, TA1535, and WP2-uvrA⁻ in the presence and absence of metabolic activation. These doses will be chosen based on inducing a range of test article toxicity and maximizing the applied dose.

Control Substances

Control substances may be prepared and used in the mutagenicity assay as described in Table 9.

TABLE 9

| Control | Strain | Concentration |
|---|---|---|
| ICR-191 Acridine | TA97a | 1.0 μg/plate |
| 2-nitrofluorene | A98 | 10.0 μg/plate |
| Sodium azide | TA100 and TA1535 | 1.5 μg/plate |
| 1-methyl-3-nitro-1- | WP2-uvrA⁻ | 4.0 μg/plate |
| nitrosognanidine | | |
| 2-aminoanthracene | all strains (except | 10.0 μg/plate |
| | TA1535) | |
| 2-aminoanthracene | TA1535 | 1.6 μg/plate |

Negative (Vehicle) Control

Tester strains will be plated with untreated dextrose solution at the corresponding maximum concentration (0.1 mL), with and without S9. These plates serve as the negative controls and provide information regarding background lawn and revertant colony formation.

Dose Range Assay

The initial dose range assay starts at the maximum concentration of 2.0 mg/plate. The four lower doses to be tested will be diluted in a 1:2 dilution series.

Reverse Mutation Assay

Each separate bacterial strain, with and without S9, is considered a separate experiment with its own concurrent positive and vehicle controls. All plates will be scored with an automated colony counter and a printout of the data was made. The positive controls will consist of direct-acting mutagens and mutagens requiring metabolic transformation. A two-fold or greater increase in reversion rates may be observed for all strains with the appropriate positive control. The negative control article reversion rates for each strain should be within or slightly below the expected ranges from laboratory historical data. An induced positive result for any strain would be demonstrated by at least a two-fold increase in the number of revertant colonies per plate over the negative control values.

### Example 16

### In Vitro Chromosome Aberration Assay in CHO Cells

The Chromosomal Aberration Assay may be one of several in vitro tests that can be used to screen materials for their potential genetic toxicity. Chromosome aberrations are mutations which have been associated with carcinogenesis. Therefore, the chromosome aberration assay is relevant for testing potential mutagens and carcinogens (Galloway et al., Environ. Mut. (1985) 7:1-51; Galloway et al., Environ. Mut. (1987) 10:1-175). This Chromosome Aberration Assay evaluates the potential of the test article extracts to induce damage in Chinese Hamster Ovary Cells (CHO). This test will be conducted in the presence and absence of an exogenous mammalian activation system (S9) over three treatment periods. All negative control treated preparations should demonstrate normal levels of spontaneously occurring aberrations while positive control treated cultures should demonstrate dramatic, dose dependent increases in aberrant chromosomes.

A representative assay to determine whether a test material is clastogenic, i.e., whether it has the capacity to break chromosomes may be designed as follows. Clastogenicity is an important endpoint because it is through chromosomal breakage and inappropriate rejoining that certain oncogenes (e.g., myc) can be activated and certain tumor suppressor genes (e.g., those suppressing retinoblastoma) can be inactivated). In this test, mammalian Chinese Hamster Ovary (CHO) cells will be exposed to the test material and blocked in metaphase using a spindle poison. Visualization of chromosomes will be performed microscopically after hypotonic swelling, fixing and staining the treated CHO cells. Agents found to be capable of inducing chromosome breakage have a high probability of being carcinogens and also have the potential for inducing heritable chromosomal defects.

The CHO-K₁ cell line (ATCC number: CCL-61) is a proline auxotroph with a modal chromosome number of 20 and a population doubling time of 10-14 hours. This system has been shown to be sensitive to the clastogenic activity of a variety of chemicals (Preston et al., Mutation Res. (1981) 87:143-188). CHO cells will be grown and maintained in McCoy's 5A medium supplemented with 10% fetal calf serum, 1% L-glutamine (2 mM), penicillin (100 units/mL), and streptomycin (100 μg/mL). Cultures will be incubated in 5-7% CO₂ with loose caps in a humidified incubator at 37±2° C.

Test Procedures

A stock solution will be prepared at 5 mg/mL. Lower doses will be prepared by dilution in 5% dextrose from this stock immediately prior to use. To minimize any chance of degradation, the test article solutions will be kept on ice after preparation and until just prior to dosing procedures. Cells will be seeded at approximately 1-1.5×10⁶ cells per 75 cm² tissue culture flask in 10 mL fresh medium one day prior to treatment. For treatment, spent medium will be replaced with fresh growth medium and the test article extract, negative or positive control will be added to each flask. Positive controls will be dosed in 0.1 mL volumes to minimize vehicle toxicity. The test article dilutions and negative control will be dosed in 1 mL volumes. Fresh medium will be added to bring the total treatment volume to 10 mL. For the portion of the test with metabolic activation, the S9 activation mix will be added to serum free medium at 1.5%, (v/v) final concentration. All treatments will be carried out in duplicate. The cells will be incubated at 37±2° C. in the presence of the test article extract, the S9 reaction mixture (metabolic activation portion of the study only) and growth medium. The assay will be divided into three treatment periods: 3 hours, 3 hours with S9 activation, and 20 hours.

After the treatment period, all flasks will be evaluated microscopically for gross manifestations of toxicity. i.e., morphological changes in cells or significant cell detachment. All flasks will be washed twice with phosphate buffered saline (PBS). Normal growth medium containing 10% fetal bovine serum (FBS) will be added to the freshly washed cells and the flasks will be returned to the incubator for an additional 14.5-15.5 hours. Microscopic evaluation will be performed immediately prior to harvest. Two hours prior to harvest, 1 μg of colcemid will be added (0.1 μg/mL final concentration) to all flasks to accumulate dividing cells.

The test article extracts will be tested in duplicate at six dose levels (0.5, 0.16, 0.05, 0.016, 0.005, and 0.0016 ml/mL final concentration in culture) along with appropriate vehicle and positive controls.

Metabolic Activation System

The use of a metabolic activation system is an important aspect for evaluation of a test article, as some compounds exist only in a promutagenic state. That is, they become mutagenic only after being acted upon by an outside metabolic source. In vitro test systems lack this ability to metabolize compounds unless an outside system such as S9 is added.

The in vitro metabolic activation system to be used in this assay may comprise Sprague Dawley rat liver enzymes and an energy producing system necessary for their function (NADP and isocitric acid; core reaction mixture). The enzymes will be contained in a preparation of liver microsomes (S9 fraction) from rats treated with Arochlor 1254 to induce enzymes capable of transforming chemicals to more active forms. The S9 may be purchased from Moltox (Boone, N.C.) and retained frozen at less than −70° C. until use. This S9 fraction will be thawed immediately before use and added to the core reaction mixture.

Cell Fixation, Staining and Scoring

Metaphase cells will be collected by mitotic shake off, swollen with 75 mM KCl, fixed in methanol: glacial acetic acid (3:1 v/v). Cells will be pipetted onto glass slides after resuspension in fresh fixative and air dried. The slides will be labeled with a blind code. Three slides will be prepared from each treatment flask. Slides will be stained with Giemsa and permanently mounted. All slides will be read under blind code with the exception of the high dose positive controls, which are evaluated first to ensure the aberration frequency was adequate. Two hundred cells per dose (100 from each of the duplicate flasks) will be read from each of the doses. One hundred cells will be read from each of the high dose positive controls in accordance with the following definitions and were scored as such.

Chromatid Type

TG (Chromatid Gap): “Tid Gap”. An achromatic (unstained) region in one chromatid, the size of which is equal to or smaller than the width of a chromatid. These are noted but not usually included in final totals of aberrations, as they may not all be true breaks.

IG (Isochromatid Gap): “Chromosome Gap”. The gaps are at the same locus in both sister chromatids. These are noted but are not usually included in final totals of aberrations, as they may not all be true breaks.

TB (Chromatid Break): An achromatic region in one chromatid, larger than the width of a chromatid. The associated fragment may be partially or completely displaced, or missing.

ID (Chromatid Deletion): Length of chromatid “cut” from midregion of a chromatid resulting in a small fragment or ring lying beside a shortened chromatid or a gap in the chromatid.

TR (Triradial): An exchange between two chromosomes, which results in a three-armed configuration. May have an associated acentric fragment.

QR (Quadriradial): The same as the triradial, but resulting in a four-armed configuration.

CR (Complex Rearrangement): An exchange among more than two chromosomes which is the result of several breaks and exchanges.

TI (Chromatid Interchange): Exchange within a chromosome involving one or both arms.

### Chromosome Type

SB (Chromosome Break): Terminal deletion. Chromosome has a clear break forming an abnormal (deleted) chromosome with an acentric fragment that is dislocated and may remain associated or may appear anywhere in the cell.

DM (Double Minute Fragment): Chromosome interstitial deletion. These appear as small double “dots” or may be paired rings. In some cases, they cannot be distinguished from acentric fragments that result from exchanges or terminal deletions.

D (Dicentric): An exchange between two chromosomes that results in a chromosome with two centromeres. This is often associated with an acentric fragment in which it is classified as Dicentric with Fragment (DF).

MC (Multi-centric Chromosome): An exchange among chromosomes that results in a chromosome with more than two centromeres.

R (Ring): A chromosome that forms a circle containing a centromere. This is often associated with an acentric fragment, in which case it is classified as Ring with Fragment (RF). Acentric rings are also included in this category.

Ab (Abnormal Monocentric Chromosome): This is a chromosome whose morphology is abnormal for the karyotype, and often the result of such things as a translocation or pericentric inversion. Classification used if abnormally cannot be ascribed to, e.g., a reciprocal translocation.

T (Translocation): Obvious transfer of material between two chromosomes resulting in two abnormal chromosomes. When identifiable, scored at “T”, not as “2 Ab”.

### Other

SD (Severely Damaged Cell): A cell with 10 or more aberrations of any type. A heavily damaged cell should be analyzed to identify the type of aberrations and may not have 10 or more, e.g., because of multiple fragments such as those found associated with a tricentric.

PU (Pulverized Chromosome): Despiralized or fragmented chromosome. This may simply be at a different stage of chromosome condensation.

P (+Pulverized Cell): More than one chromosome, up to the whole nucleus, is “pulverized”.

PP (Polyploid Cell): A cell containing multiple copies of the haploid number of chromosomes. Polyploid cells are occasionally observed in normal bone marrow or cell culture. These are recorded but are not included in final totals of structural aberrations.

Control Substances

Control substances are prepared and used in this assay as described in published reports. Positive controls which may be used are: cyclophosphamide—High dose 15 μg/mL; cyclophosphamide—Low dose 5 μg/mL; mitomycin C—High dose 1.0 μg/mL; and citomycin C—Low dose 0.25 μg/mL. For negative (vehicle) control, the CHO cells are treated with the 5% dextrose negative controls with and without S9 activation. These treatments provide information regarding background numbers of aberrant cells.

Assay Validity Evaluation and Statistical Analysis

The total number of aberrations (% CA) of the solvent control culture(s) should fall within 1-14%. High dose positive controls should produce a statistically significant increase in the number of aberrations at the 95% confidence level (p<0.05) as determined by statistical analysis. Analysis of Variance (ANOVA) may be used to identify significant differences between positive and negative control groups or test article and negative control groups. A difference is considered significant when the p value obtained is less than 0.05.

### Example 17

### Safety and Tolerance Determination in Dogs

A representative study for determining the safety and tolerance of compounds at dose levels administered intravenously once daily to beagle dogs for five consecutive days, for example, may be designed as follows. Safety parameters will be monitored through observation, clinical pathology, and microscopic histopathology assessments.

Experimental Design

Table 10 summarizes a representative study. For example, the study will be conducted using three (3) test article and one (1) control article group. The control article will be the solution (5% dextrose in water) used to dilute the test article prior to administration and will be administered at the same volume as the high dose. The test article dosage levels for this study will be approximately 12, 3.8, and 1.2 mg/kg. Test and control articles will be administered once by intravenous (IV) infusion over approximately a one hour period on five consecutive days.

Blood samples for test article blood level analysis will be taken as follows (i.e., pk/tk sampling). Approximately 1.0 mL of blood will be taken from three male and three female dogs in the low dose group at approximately 20 minutes and 40 minutes from the start of the infusion, and then at the end of infusion (Time 0) and at 5, 10, 15, and 30 minutes, and 1, 2, 4, 8, 12, and 24 hours from the end of the infusion after the first and fifth doses. Also, prior to and immediately after Dose 1 and after Dose 5 for all animals, and for recovery animals prior to necropsy, approximately 5-10 second ECG tracings in a lead II configuration will be obtained. Animals will be terminated one (1) or 15 days after the last dose. Blood for hematology and clinical chemistry analysis will be drawn pre-dose and prior to euthanasia at termination. Following euthanasia, a necropsy will be performed to include collection of major organs for microscopic evaluation.

TABLE 10

| | | | PRIMARY | |
|---|---|---|---|---|
| | | | NO. | RECOVERY |
| | | | ANIMALS | (15 DAY) |
| GROUP | | DOSAGE | (MALE/ | NO. ANIMALS |
| NO. | ARTICLE^{a} | (MG/KG) | FEMALE) | (MALE/FEMALE) |
| 1 | Control | 0.0 | 3/3 | 1/1 |
| 2 | Test Article | 12.0 | 3/3 | 1/1 |
| 3 | Test Article | 3.8 | 3/3 | 1/1 |
| 4 | Test Article | 1.2 | 3/3 | 1/1 |

^{a}Delivered as an approximate 1 hour infusion

Test Methods

In a representative study, animals will be assigned to groups as follows: The heaviest dog for a sex will be assigned to Group 1, the next heaviest for that sex will be assigned to Group 2, the next heaviest to Group 3, the next heaviest to Group 4, then continue with Groups 2, 3, 4, and 1, then Groups 3, 4, 1, and 2, continuing with this pattern until each group had a full complement of animals. The test and control article will be administered at each dosing as an intravenous infusion into a cephalic or saphenous vein over approximately one hour.

Animals will be weighed daily prior to dosing and prior to necropsy. All animals will be observed for signs of pharmacological activity, behavioral changes, and toxicity immediately and one hour after dosing. Recovery animals will be also observed once daily during the recovery period. Prior to and immediately after Doses 1 and 5 for all animals, and for recovery animals prior to necropsy, approximately five second ECG tracings in a lead II configuration will be obtained. These tracings will be used to provide data for interpretation of the rhythm and amplitude changes of the QRS-complex and T-wave and to measure QT intervals on a number of segments per tracing (approximately 5-10).

Blood Collection

PK/TK. Blood samples for test article blood level analysis will be taken. Approximately 1 mL of blood will be taken from three males and three females in the low dose group at approximately 20 minutes and 40 minutes from the start of the infusion, and then at the end of infusion (Time 0) and at 5, 10, 15, and 30 minutes, and 1, 2, 4, 8, 12, and 24 hours from the end of the infusion after the first and fifth dose. Plasma (lithium heparin anticoagulant) samples will be prepared for analysis.

Clinical Pathology. After overnight fasting and prior to the first dose (baseline; all animals) and then prior to each necropsy, blood samples will be taken for hematology and clinical chemistry. For hematology assays, blood collected at baseline and prior to necropsy (fasted) are analyzed for erythrocyte count, hematocrit, MCH, leukocyte count, differential WC, MCHC, hemoglobin, MCV, platelet count, PT, and APTT. For clinical chemistry assays, blood collected at baseline and prior to necropsy (fasted) will be tested for: aspartate aminotransferase (ASP), globulin & A/G ratio, Alanine aminotransferase (ALT), sodium, alkaline phosphatase, potassium, gamma glutamyltransferase (GGT), chloride, glucose, calcium, blood urea nitrogen (BUN), total bilirubin, creatinine, inorganic phosphorus, total protein, cholesterol, albumin, and triglycerides.

Necropsy

Following blood sample collection, primary treatment and recovery group animals will be sacrificed at their respective termination times and are necropsied. Major organs will be collected, weighed, and preserved for microscopic evaluation. Necropsy will include examination of the cranial, thoracic, abdominal and pelvic cavities, their viscera, the tissues, organs, and the carcass.

Statistical Methods

Statistical analysis of the clinical chemistry and hematology values and organ and body weight data will be performed to compare the test article groups to the control group. The statistical methods used for the data will be selected as appropriate: parametric data will be analyzed using a one way Analysis of Variance, non-parametric data will be analyzed using the Kurskai-Wallis test. A paired t-test will also be used to compare baseline and post treatment clinical chemistry and hematology values for each animal. Probability (p) values of 0.05 or less will be considered significant for all statistical tests.

### Example 18

### Safety and Tolerance Study in Rats

A representative study to determine the safety and tolerance of a test compound, for example, at three dose levels administered intravenously once daily to rats for five consecutive days may be designed as follows. Safety parameters will be monitored through observation, clinical pathology, and microscopic histopathology assessments. Selected animals will also undergo blood sample collection for pharmacokinetic/toxicokinetic evaluation.

Experimental Methods

Table 11 summarizes a representative study. The study will be conducted using three (3) test and one (1) control article groups. The high and low test article groups and the control group will consist of 28 animals each and will be used to assess tolerance. The medium test article group will consist of 64 animals, of which 28 animals will be used to assess tolerance and 36 animals will be used to determine the level of test article in the blood at various time points after the first and fifth doses in the PK/TK portion of the study. The control article will be the solution (5% dextrose in water; D5W) used to dilute the test article prior to administration and is administered at the same volume as the high dose test article group. The test article dosage levels for this study will be 24, 7.6, and 2.4 mg/kg. Test and control articles will be administered by intravenous (IV) injection into a tail vein over one minute on five consecutive days.

Blood samples for test article blood level analysis will be taken as follows. Approximately 0.3-0.5 mL of blood will be taken from three male and three female rats under anesthesia at each sample time point of pre-dose and at the end of injection (Time 0) and at approximately 0.08, 0.25, 0.5, 1, 2, 4, 8, 12, and 24 hours from the end of the injection after the first and fifth doses. Animals used to assess tolerance will be terminated one day (for the primary group) or 15 days (for the recovery group) after the last dose. At termination of the tolerance test animals, blood for hematology and clinical chemistry analysis will be drawn prior to euthanasia and following euthanasia. A necropsy will be performed to include collection of major organs for microscopic evaluation. The animals used for the pk/tk blood sampling only to determine the level of test article will be euthanized after the final blood sample is collected without any further sampling or observations.

TABLE 11

| | | | PRIMARY | |
|---|---|---|---|---|
| | | | NO. | RECOVERY |
| | | | ANIMALS | (15 DAY) |
| GROUP | | DOSAGE | (MALE/ | NO. ANIMALS |
| NO. | ARTICLE^{a} | (MG/KG) | FEMALE) | (MALE/FEMALE) |
| 1 | Control | 0.0 | 3/3 | 1/1 |
| 2 | Test Article | 12.0 | 3/3 | 1/1 |
| 3 | Test Article | 3.8 | 3/3 | 1/1 |
| 4 | Test Article | 1.2 | 3/3 | 1/1 |

^{a}Delivered as an approximate 1 hour infusion

Test Methods

The test and control article will be administered at each dosing as an intravenous infusion into a tail vein over approximately one minute Animals will be weighed daily prior to dosing and prior to necropsy. All animals will be observed for signs of pharmacological activity, behavioral changes, and toxicity immediately and one hour after dosing. Recovery animals will also be observed once daily during the recovery period. The control animals will be dosed with approximately 6 mL/kg of D5W. The high, mid, and low dose test article animals will be administered dosages of approximately 24 mg/kg, 7.6 mg/kg, and 2.4 mg/kg, respectively.

Blood Collection

PK/TK. Blood samples for test article blood level analysis will be taken. Utilizing 18 male and 18 female medium dose animals, approximately 0.3-0.5 mL of blood will be taken from three male and three female rats under anesthesia at each sampling time point of pre-dose and at the end of injection (Time 0), and at approximately 0.08, 0.25, 0.5, 1, 2, 4, 8, 12, and 24 hours from the end of the injection after the first and fifth dose. Blood sampling will be via retro-orbital bleeding or cardiac puncture bleeding for an animal's terminal sample. Plasma (lithium heparin anticoagulant) samples will be prepared for analysis. General procedures for chemical pathology, necropsy, and histopathology, as well as statistical methods, such as those previously described, will be followed.

### Example 19

### Phosphorylated and Total p53 Assay Protocol

A phosphorylated and total p53 assay protocol may be designed as follows. On Day 1, cells are seeded at 2×10⁶ cells/10 cm dish/10 mL medium. On day two, cells will be treated as follows: control=0.05% DMSO (5 μl DMSO stock/10 ml medium); 1 μM test compound (1 μl Stock (10 mM)/10 ml medium); 2 μM test compound (2 μl Stock (10 mM)/10 ml medium); 3 μM test compound (3 μl Stock (10 mM)/10 ml medium); 4 μM test compound (4 μl Stock (10 mM)/10 ml medium) and 5 μM test compound (5 μl Stock (10 mM)/10 ml medium).

On Day 3, cells will be harvested and attached and floating cells will be collected. Cells will be washed twice with PBS, counted and collected at 4×10⁶ cells/sample. The cell pellet will be frozen at −80° C. until further use. On the same day or on Day 4, cells will be extracted using a cell extraction buffer (3 mL cell extraction buffer, 300 μl protease inhibitor and 10 μl 0.3M PMSF). To each sample will be added 200 μl Buffer, and the solution will be vortexed and set on ice for 30 minutes, and subsequently vortexed after every 10 mins. The solution will be then centrifuged at 13,000 rpm for 10 min, and 100 μl supernatant per tube will be aliquoted and stored at −80° C.

Assay preparation (Day 5). An anti-rabbit IgG HRP solution will be prepared by diluting 10 μl of 100× concentrate solution with 1 ml HRP diluent for each 8-well strip. A wash buffer solution will be prepared by diluting the original vial (×25) using distilled water to make a ×1 solution. Dilutions of p53 standard solution or p53 total solution can be prepared as described according to representative parameters of Table 12. To ensure complete reconstitution, standard 1 will be mixed gently and allowed to sit for 10 minutes at room temperature.

TABLE 12

| | Conc. | Standard Soln. | Dilution Buffer |
|---|---|---|---|
| Standard 1 | 100 Units/ml | Reconstitute 1 Vial worth 0.7 ml | |
| | | of standard Dil. Buffer | |
| Standard 2 | 50 Units/ml | 250 μl of Standard 1 | 250 μl |
| Standard 3 | 25 Units/ml | 250 μl of Standard 2 | 250 μl |
| Standard 4 | 12.5 Units/ml | 250 μl of Standard 3 | 250 μl |
| Standard 5 | 6.25 Units/ml | 250 μl of Standard 4 | 250 μl |
| Standard 6 | 3.12 Units/ml | 250 μl of Standard 5 | 250 μl |
| Standard 7 | 1.6 Units/ml | 250 μl of Standard 6 | 250 μl |
| Standard 8 | 0 | | 250 μl |

Test Procedure. Allow all solution to reach RT and mix gently before use. Take out and insert 8-well strips. Add 100 μl of standard dilution buffer to standard 8 well (0 ng/ml/well or 0 Units/well). Add nothing to the chromogen blank well. Add 100 μl of standard or diluted sample to the appropriate microtiter wells. Generally, the sample should be diluted with standard dilution buffer at least 1:10 or greater. Each sample will be run in duplicates. Gently tap the side of the plate to thoroughly mix. Cover plate with plate cover and incubate for 2 hours at RT or o/n at 4 C. Wash wells with 400 μl working wash buffer 4 times. Let soak for 15-30 sec., and then aspirate the liquid. After washing, the plate will be inverted and tapped dry on absorbance tissue. Add 100 μl of anti-p53 [015] or anti-p53 (total) (detection antibody) to each well except chromogen blank. Tap gently to mix; cover plate and incubate 1 hour at RT. Aspirate solution from wells thoroughly.

Wash wells with 400 μl working wash buffer four times. Let soak for 15-30 sec., and then aspirate the liquid. After washing, the plate will be inverted and tapped try on absorbance tissue. Add 100 μl of anti-rabbit IgG HRP working solution. to each well except chromogen blank. Cover plate and incubate 30 min at RT. Wash wells with 400 μl working wash buffer four times. Let soak for 15-30 sec., and then aspirate the liquid. After washing, the plate will be inverted and tapped try on absorbance tissue. Add 100 μl of TMB (stabilized chromogen substrate) to each well and incubate for 30 min. at RT in the dark. The color will change to blue. Add 100 μl Stop soln. Tap plate gently to mix. The color should change to yellow. Read the plate at A450 nm by setting chromogen blank (=100 μl TMB+100 μl Stop soln) as blank. Read absorbance within 2 hours of assay completion.

### Example 20

### Caspase-3/7 Assay Protocol

A representative Caspase-3/7 assay protocol may be designed as follows. On Day 1, seed 0.015×10⁶ HCT-116 cells/50 ul/well. Incubate o/n in 37° C. CO₂ incubator. On Day 2, remove 25 ul of medium from wells. Treat HCT-116 cells with 1, 3, and 5 uM test compound. Treat positive control group with Staurosporin 0.01, 0.1, 1 uM. Keep six negative control wells treated with medium only (add 25 ul of diluted sample to appropriate wells). Incubate for 24 h at 37° C. in a CO₂ incubator. On Day 3, prepare Apo-ONE Homogeneous Caspase-3/7 assay reagent (Promega) at 10 ul reagent/1 ml buffer. Add 50 ul of diluted reagent. Incubate one hour at room temp. Measure fluorescence at 485/520.

### Example 21

### DNA Cell Cycle Analysis Protocol

A representative DNA cell cycle analysis protocol will be designed as follows. Seed 1.5-2.0×10⁶ cells/10 cm dish (seed one extra dish for unstained cells). Incubate cells in 37° C. humidified 5% CO₂ incubator for 24 hours. For synchronizing cells in a low growth state to make cells quiescent, remove media and rinse once with serum-free media, add 10 ml of serum-free media to each dish. Incubate the cells for 24 hr in a 37° C. humidified 5% CO₂ incubator. Remove media and add treatment (diluted in serum contained media, 10 ml): 1-5 μM test compound plus control. Incubate the cells for 24 hr in a 37° C. humidified 5% CO₂ incubator.

To trypsinize/isolate cells, remove treatment. Add 3 ml trypsin/EDTA solution. Keep floating cells and combine with attached cells. Incubate for 5 min in a 37° C. humidified 5% CO₂ incubator. Add 3 ml media (containing FBS) to wells and pipette into centrifuge tube. Centrifuge at 1000 rpm for 5 minutes. Decant supernatant and re-suspend pellet in 2-3 ml PBS. Count cells and wash cells once by putting 2×10⁶ cells/tube, adding 2 ml PBS and centrifuging at 1000 rpm for 5 minutes. Re-suspend pelleted cells in 0.3 ml cold PBS.

To fix cells, gently add 0.7 ml ice cold 70% ethanol drop wise to tube containing 0.3 ml of cell suspension in PBS while vortexing. Leave on Ice for one hour (or up to a few days at 4 C). Centrifuge at 1000 rpm for 5 minutes. Wash one time with cold PBS (1-2 ml). Centrifuge at 1000 rpm for 5 minutes. Re-suspend cell pellet in 0.25 ml cold PBS, add 5 μl of 10 mg/ml RNAse A (the final concentration being 0.2-0.5 mg/ml). Incubate at 37 C for 1 hour. Add 10 μl of 1 mg/ml of propidium iodide solution in deionized water (the final concentration being 10 μl/ml), and keep in the dark and at 4° C. until analysis. Analyze on FACS by reading on cytometer at 488 nm Cells may be stained with propidium iodide on the same day of analysis.

It is understood that the foregoing detailed description and accompanying examples are merely illustrative, and are not to be taken as limitations upon the scope of the invention. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof. U.S. patents and publications referenced herein are incorporated by reference.

What is claimed is:

1. A compound having formula (1): 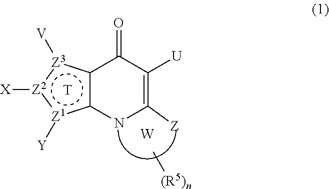
and pharmaceutically acceptable salts thereof;
wherein V, X, and Y are absent if attached to a heteroatom other than Nitrogen, and independently H, halo, azido, R², CH₂R², SR², OR² or NR¹R² when attached to C or N; or
wherein V and X, or X and Y may form a carbocyclic ring, heterocyclic ring, aryl or heteroaryl, each of which may be optionally substituted and/or fused with a cyclic ring;
Z¹, Z² and Z³ are C, N, O or S; wherein at most one of Z¹, Z² and Z³ is O, and at most one of Z¹, Z² and Z³ is S, and at most two of Z¹, Z² and Z³ are C;
Z is O, S, NR², CH₂ or C═O;
W together with N and Z forms an optionally substituted 5- or 6-membered ring that is fused to an optionally substituted aryl or heteroaryl, wherein said aryl or heteroaryl may be monocyclic or fused with a single or multiple ring, and wherein said ring optionally contains a heteroatom;
U is C(O)R², C(O)OR², C(O)NR¹R², C(O)NR¹—(CR¹₂)ₙ—NR³R⁴, SO₃R², SO₂NR¹R² or SO₂NR¹—(CR¹₂)ₙ—NR³R⁴;
wherein in each NR¹R², R¹ and R² together with N may form an optionally substituted ring;
in NR³R⁴, R³ and R⁴ together with N may form an optionally substituted ring;
R¹ and R³ are independently H or C₁₋₆ alkyl;
each R² is H, or a C₁₋₁₀ alkyl or C₂₋₁₀ alkenyl each optionally substituted with a halogen, one or more non-adjacent heteroatoms selected from N, O and S, a carbocyclic ring, a heterocyclic ring, an aryl or heteroaryl, wherein each ring is optionally substituted; or R² is an optionally substituted carbocyclic ring, heterocyclic ring, aryl or heteroaryl; or R² is COR¹ or S(O)ₓ—R¹ wherein x is 1-2;
R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;
each R⁵ is a substituent at any position on W; and is H, OR², amino, alkoxy, amido, halogen, cyano or an inorganic substituent; or R⁵ is C₁₋₆ alkyl, C₂₋₆ alkenyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring, optionally fused to an additional optionally substituted carbocyclic or heterocyclic ring; and
n is 1-6.

2. The compound of claim 1, wherein T forms an optionally substituted 5-membered ring selected from the group consisting of:

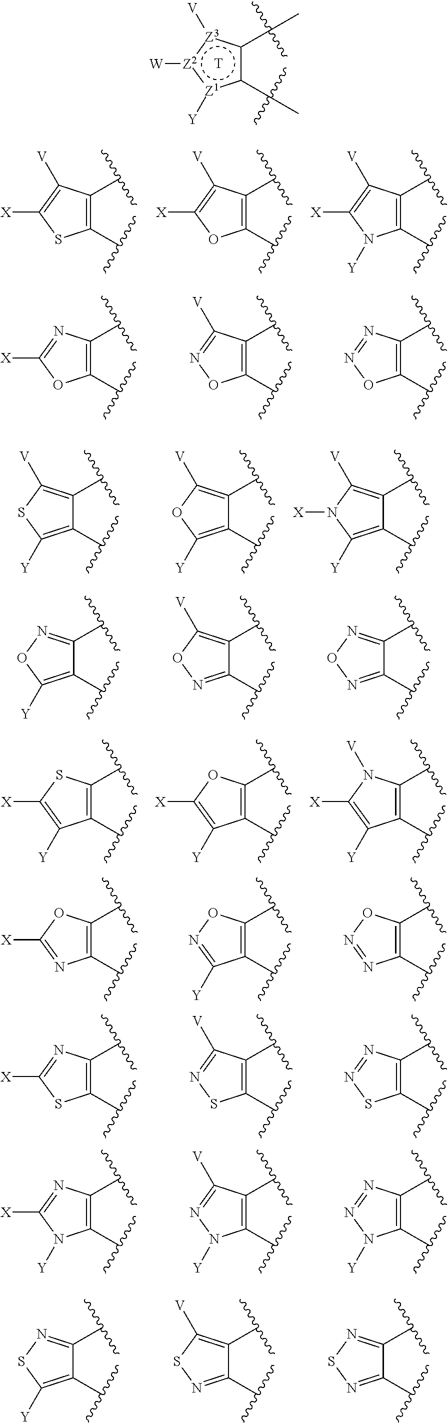

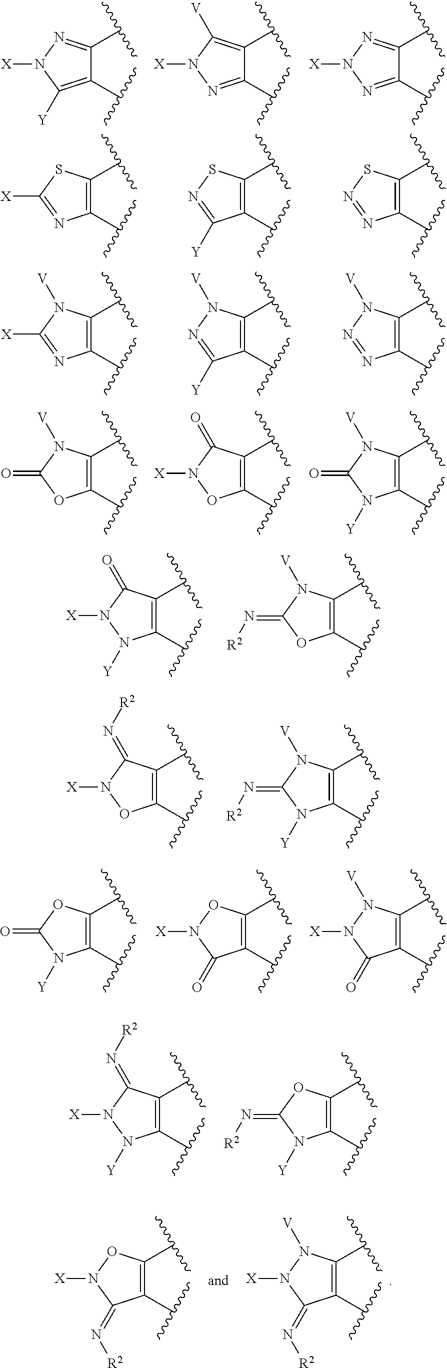

3. The compound of claim 1, wherein W together with N and Z form an optionally substituted 5- or 6-membered aryl or heteroaryl ring that is fused to an optionally substituted aryl or heteroaryl selected from the group consisting of: 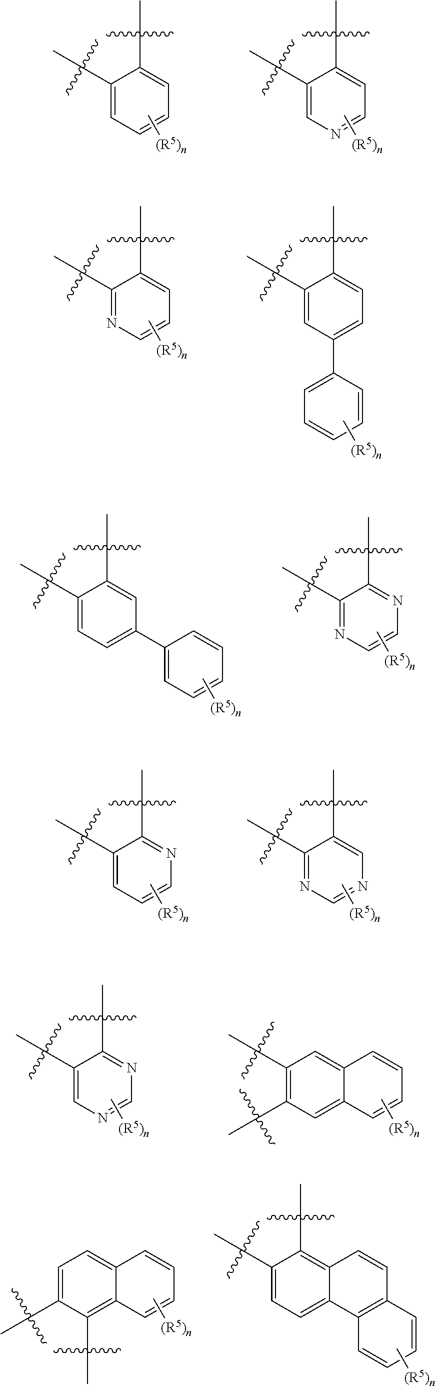 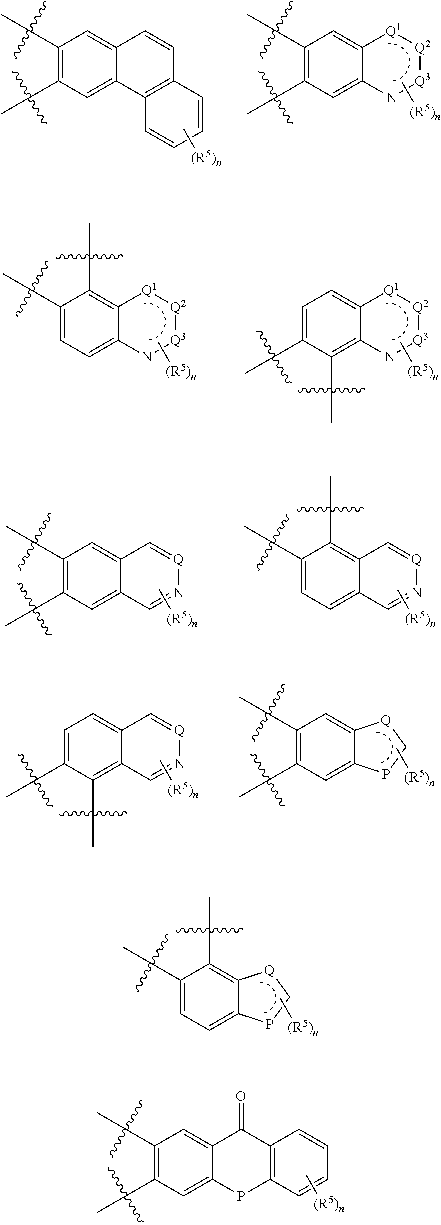 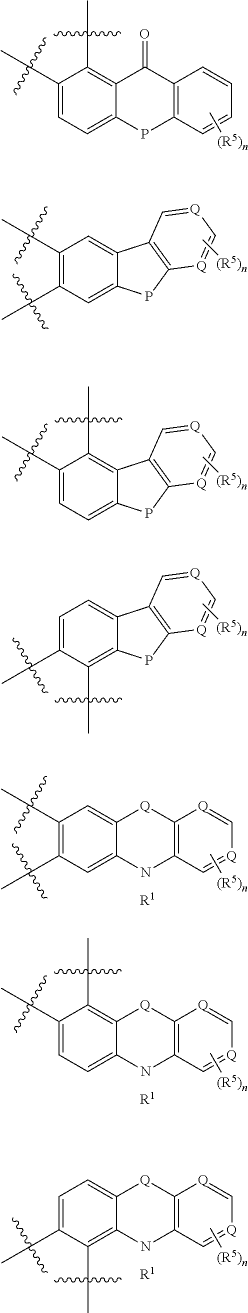 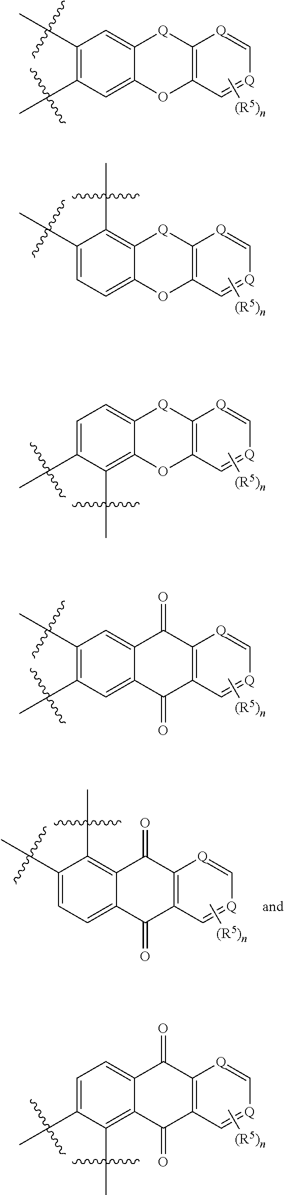
wherein each Q, Q¹, Q², and Q³ is independently CH or N;
P is independently O, CH, C═O or NR¹;
n and R⁵ is as defined above.

4. The compound of claim 1, wherein W together with N and Z form a group having the formula selected from the group consisting of 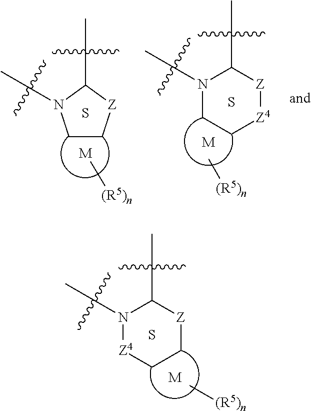
wherein Z is O, S, NR², CH₂ or C═O;
each Z⁴ is CR⁶, NR², or C═O;
R⁶ is H, or a substituent known in the art, including but not limited to hydroxyl, alkyl, alkoxy, halo, amino, or amido; and
Ring S and M may be saturated or unsaturated.

5. The compound of claim 1, wherein W together with N and Z forms a 5- or 6-membered ring that is fused to a phenyl.

6. The compound of claim 1, having the general formula (2A) or (2B): 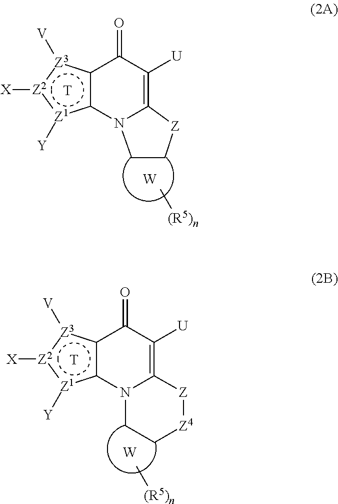
wherein U, V, W, X, Y, Z, Z¹, Z², Z³, R⁵ and n are as described in formula (1);
Z⁴ is CR⁶, NR², or C═O;
Z and Z⁴ may optionally form a double bond.

7. The compound of claim 1, having the general formula (3): 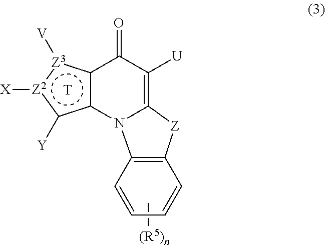
wherein U, V, X, Y, Z, Z¹, Z², Z³, R⁵ and n are as described in formula 1.

8. The compound of claim 1, having the general formula (4A) or (4B): 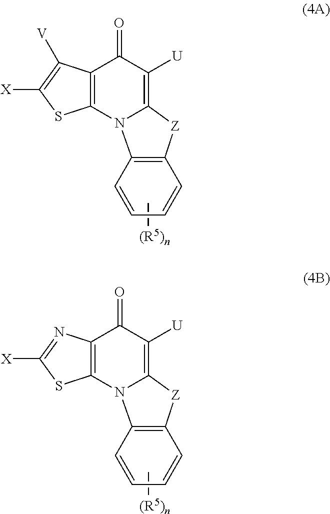
wherein U, V, X, Z, R⁵ and n are as described above.

9. The compound of claim 1, wherein U is C(O)NR¹R²;
R¹ is H, and R² is a C₁₋₁₀ alkyl optionally substituted with a heteroatom, or an optionally substituted C₃₋₆ cycloalkyl, aryl or a 5-14 membered heterocyclic ring containing one or more N, O or S.

10. The compound of claim 9, where U is C(O)NR¹R², wherein R² is a C₁₋₁₀ alkyl substituted with an optionally substituted morpholine, thiomorpholine, imidazole, aminodithiadazole, pyrrolidine, piperazine, pyridine or piperidine ring.

11. The compound of claim 9, where U is C(O)NR¹R², where in R¹ and R² together with N form an optionally substituted piperidine, pyrrolidine, piperazine, morpholine, thiomorpholine, imidazole, or aminodiathiazole.

12. The compound of claim 1, wherein U is C(O)NR¹—(CR¹₂)ₙ—NR³R⁴; n is 1-4; and R³ and R⁴ in NR³R⁴ together form an optionally substituted piperidine, pyrrolidine, piperazine, morpholine, thiomorpholine, imidazole, or aminodiathiazole.

13. The compound of claim 1, wherein U is C(O)NH—(CH₂)ₙ—NR³R⁴; and R³ and R⁴ together with N form an optionally substituted pyrrolidine, which may be linked to (CH₂)ₙ at any position in the pyrrolidine ring.

14. The compound of claim 13, wherein R³ and R⁴ together with N form an N-methyl substituted pyrrolidine.

15. The compound of claim 14, wherein U is C(O)NH—(CH₂)₂-(1-methylpyrrolidin-2-yl) or C(O)NH—(CH₂)₂-(2-pyrrolidin-1-yl).

16. The compound of claim 1, wherein Z is S or NR².

17. The compound of claim 1, wherein at least one of V, X or Y when attached to C is halo.

18. The compound of claim 1, wherein each optionally substituted moiety may be substituted with one or more acetyl, OR², amino, alkoxy, amido, halogen, cyano, an inorganic substituent; or a C₁₋₆ alkyl, C₂₋₆ alkenyl, C₂₋₆ alkynyl, —CONHR¹, each optionally substituted by halo, an oxo group, aryl or one or more heteroatoms; inorganic substituents, aryl, carbocyclic or a heterocyclic ring.

19. The compound of claim 1, wherein two of Z¹, Z² and Z³ are C and the other is N, O or S.

20. The compound of claim 19, wherein Z¹ is S.

21. The compound of claim 1, wherein two of Z¹, Z² and Z³ are selected from N, O and S, and the other is C.

22. A pharmaceutical composition comprising the compound of claim 1, and a pharmaceutically acceptable carrier.

23. A method for ameliorating a tumor or cancer, comprising administering to a system or a subject in need thereof an effective amount of the compound of claim 1 or a pharmaceutical composition thereof and optionally with a procedure and/or a chemotherapeutic agent, thereby reducing cell proliferation and/or ameliorating said cell-proliferative disorder.

24. The method of claim 23, wherein said cell proliferative disorder is a tumor or cancer.

25. The method of claim 23, wherein the compound of claim 1 is administered to a subject in need thereof, and said subject is human or an animal.

26. A method for reducing microbial titers and/or ameliorating a microbial infection, comprising contacting a system or a subject in need thereof with an effective amount of the compound of claim 1 or a pharmaceutical composition thereof and optionally with an antimicrobial agent, thereby reducing microbial titers and/or ameliorating said microbial infection.

27. The method of claim 26, where said system is a cell or tissue, and said subject is human or an animal.

28. The method of claim 26, wherein the microbial titers and/or microbial infection are viral, bacterial or fungal titers.

29. A method for inducing cell death and/or inducing apoptosis, comprising administering to a system or a subject in need thereof an effective amount of a composition comprising a compound in claim 1, or a pharmaceutical composition thereof and optionally with a procedure and/or a chemotherapeutic agent, thereby inducing cell death and/or inducing apoptosis.

30. The method of claim 29, wherein said system is a cell or tissue, and said subject is human or an animal.

31. The method of claim 29, wherein said procedure is radiotherapy or a surgical procedure.

32. The compound of claim 1, selected from the group consisting of 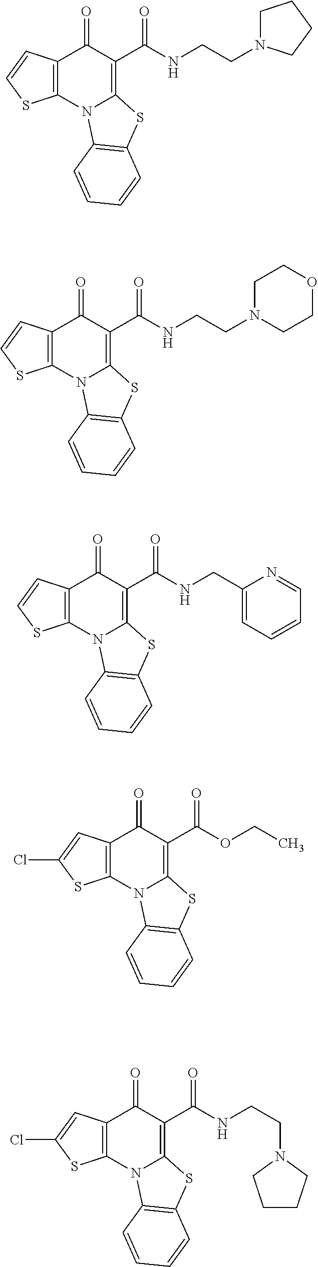 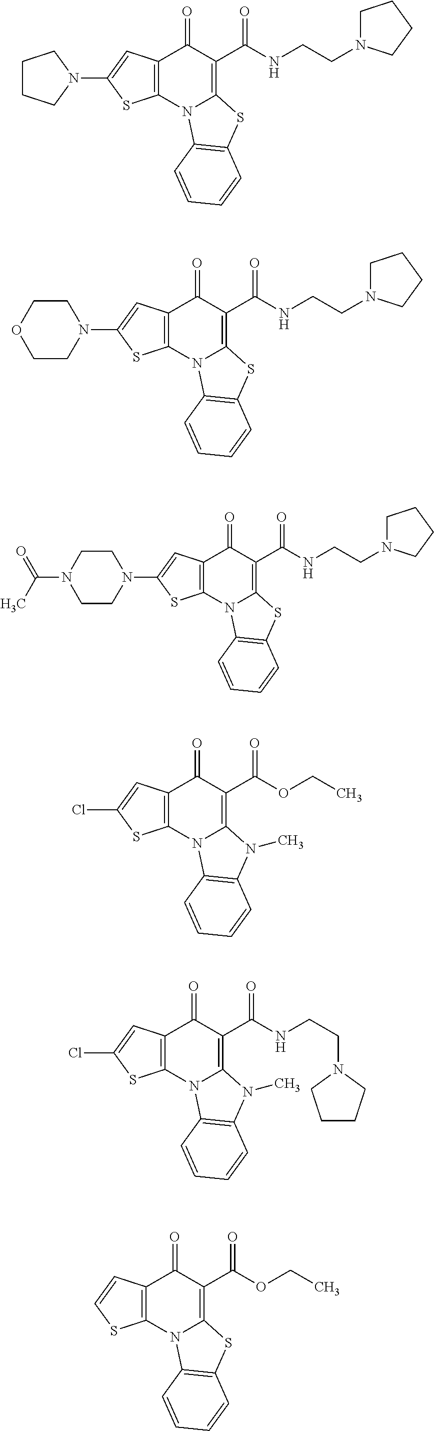 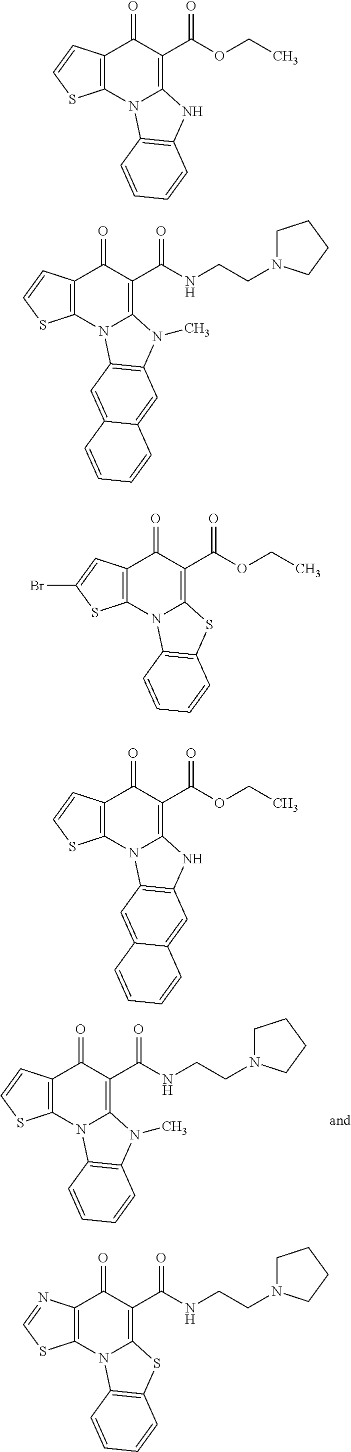
and the pharmaceutically acceptable salts thereof.

34. A composition comprising a compound of claim 1 in combination with a protein kinase.

35. A composition comprising a compound of claim 1 in combination with a nucleic acid containing a nucleotide sub-sequence from SEQ ID NO: 1, a complement thereof, or RNA transcript of the foregoing.

36. The compound of claim 21, wherein Z¹ is S, Z² is C and Z³ is N.
